(19) United States
(12) Patent Application Publication
(10) Pub. No.: US 2026/0258499 A1
(43) Pub. Date: Sep. 3, 2026

(54) Treatment Of Psoriasis With Interferon Induced Helicase C Domain 1 (IFIH1) Inhibitors

(71) Applicant: Regeneron Pharmaceuticals, Inc., Tarrytown, NY (US)

(72) Inventors: Julie E. Horowitz, Tarrytown, NY (US); Manuel Allen Revez Ferreira, Tarrytown, NY (US); Katherine Siminovitch, Tarrytown, NY (US); Aris Baras, Tarrytown, NY (US); Lori Khrimian, Tarrytown, NY (US); Katia Karalis, Tarrytown, NY (US)

(21) Appl. No.: 19/544,239

(22) Filed: Feb. 19, 2026

Related U.S. Application Data
(63) Continuation of application No. 17/836,735, filed on Jun. 9, 2022, now Pat. No. 12,584,174.
(60) Provisional application No. 63/209,075, filed on Jun. 10, 2021.

(51) Int. Cl. C12Q 1/6883; A61K 31/7105; A61K 38/46; A61K 45/06; C12N 15/113

(52) U.S. Cl. CPC C12Q 1/6883; A61K 31/7105; A61K 38/465; A61K 45/06; C12N 15/1138; C12N 2310/11; C12N 2310/14; C12N 2310/531; C12N 2320/31; C12Q 2600/118; C12Q 2600/156

(57) ABSTRACT

The present disclosure provides methods of treating subjects having psoriasis, and methods of identifying subjects having an increased risk of developing psoriasis.

### REFERENCE TO SEQUENCE LISTING

This application includes a Sequence Listing submitted electronically as an XML file named 381204548SEQ, created on Jan. 8, 2026, with a size of 190,372 bytes. The Sequence Listing is incorporated herein by reference.

### FIELD

The present disclosure relates generally to the treatment of subjects having psoriasis with Interferon Induced Helicase C Domain 1 (IFIH1) inhibitors and/or Tripartite Motif Containing 65 (TRIM65) inhibitors, and methods of identifying subjects having an increased risk of developing psoriasis.

### BACKGROUND

Psoriasis is an autoimmune skin disease marked by the presence of small elevations of the skin as well as silvery scales. These psoriatic lesions occur most often on the elbows, knees, trunk and scalp. In the area where scales have been shed, tiny bleeding points called “Auspitz sign” appear. The major pathophysiological events involved in the disease process are accelerated epidermal proliferation and metabolic activity, proliferation of capillaries in the dermal region, and invasion of the dermis and epidermis by inflammatory cells. Coal tar and salicylic acid are the only two Category I drugs mentioned in the Final Monograph for dandruff, seborrheic and psoriatic drugs. There are a number of prescription products that are also useful, such as theophylline, which arrests the proliferation of cells during the metaphase stage of cell division.

Psoriasis is believed to be a genetic disease that is triggered by environmental factors. For example, symptoms often worsen during winter and with certain medications, such as beta blockers or NSAIDs. Infections and psychological stress may also be triggers. These periods of increased disease are called flare-ups. There are five main types of psoriasis: plaque, guttate, inverse, pustular, and erythrodermic. Plaque psoriasis, also known as psoriasis vulgaris, is the most common, making up about 90 percent of cases. It typically presents as red patches with white scales on top. Areas of the body most commonly affected are the back of the for psoriasis, shins, navel area, and scalp. Guttate psoriasis has drop-shaped lesions. Pustular psoriasis presents as small non-infectious pus-filled blisters. Erythrodermic psoriasis occurs when the rash becomes very widespread, and can develop from any of the other types. In most affected people, changes in color of fingernails and toenails often occur.

The underlying mechanism of psoriasis involves the immune system reacting to skin cells. Skin cells are replaced every three to five days in psoriasis rather than the usual 28 to 30 days. These changes are believed to stem from the premature maturation of keratinocytes induced by an inflammatory cascade in the dermis involving dendritic cells, macrophages, and T cells. These immune cells move from the dermis to the epidermis and secrete inflammatory chemical signals (cytokines) such as interleukins, tumor necrosis factor-a, interleukin-1b, interleukin-6, and interleukin-22. These secreted inflammatory signals are believed to stimulate keratinocytes to proliferate.

Interferon Induced Helicase C Domain 1 (IFIH1) encodes an innate immune receptor which acts as a cytoplasmic sensor of viral nucleic acids and plays a major role in sensing viral infection and in the activation of a cascade of antiviral responses including the induction of type I interferons and proinflammatory cytokines. Its ligands include mRNA lacking 2′-O-methylation at their 5′ cap and long-dsRNA (>1 kb in length). Upon ligand binding, IFIH1 associates with mitochondria antiviral signaling protein (MAVS/IPS1) which activates the IKK-related kinases TBK1 and IKBKE which phosphorylate interferon regulatory factors IRF3 and IRF7, which in turn activate transcription of antiviral immunological genes, including interferons (IFNs), IFN-alpha and IFN-beta. IFIH1 is responsible for detecting the Picornaviridae family members such as encephalomyocarditis virus (EMCV) and mengo encephalomyocarditis virus (ENMG). IFIH1 also plays an important role in amplifying innate immune signaling through recognition of RNA metabolites that are produced during virus infection by ribonuclease L (RNase L). IFIH1 may play a role in enhancing natural killer cell function and may be involved in growth inhibition and apoptosis in several tumor cell lines.

Tripartite Motif Containing 65 (TRIM65) is an E3 ubiquitin ligase and a regulator of a variety of cellular processes, as well as tumor progression. By sequence consensus, TRIM65 belongs to the tripartite motif family. TRIM65 was originally identified as a gene with SNPs associated with cerebral white matter lesions, but was later shown to be a cofactor for the regulation of microRNA function. TRIM65 regulates microRNA activity by ubiquitination of TNRC6 while establishing its E3 ubiquitin ligase activity. Like other TRIM members that are involved in the immune response, TRIM65 also participates in the virus-induced innate immune response by ubiquitination of substrate proteins.

### SUMMARY

The present disclosure provides methods of treating a subject having psoriasis, the methods comprising administering an IFIH1 inhibitor to the subject.

The present disclosure also provides methods of treating a subject having guttate psoriasis, the methods comprising administering an IFIH1 inhibitor to the subject.

The present disclosure also provides methods of treating a subject having plaque psoriasis, the methods comprising administering an IFIH1 inhibitor to the subject.

The present disclosure also provides methods of treating a subject having inverse psoriasis, the methods comprising administering an IFIH1 inhibitor to the subject.

The present disclosure also provides methods of treating a subject having pustular psoriasis, the methods comprising administering an IFIH1 inhibitor to the subject.

The present disclosure also provides methods of treating a subject having erythrodermic psoriasis, the methods comprising administering an IFIH1 inhibitor to the subject.

The present disclosure also provides methods of treating a subject with a therapeutic agent that treats or inhibits psoriasis, wherein the subject has psoriasis, the methods comprising the steps of: determining whether the subject has an IFIH1 missense variant nucleic acid molecule encoding an IFIH1 predicted loss-of-function polypeptide by: obtaining or having obtained a biological sample from the subject; and performing or having performed a sequence analysis on the biological sample to determine if the subject has a genotype comprising the IFIH1 missense variant nucleic acid molecule; and administering or continuing to administer the therapeutic agent that treats or inhibits psoriasis in a standard dosage amount to a subject that is IFIH1 reference, and/or administering an IFIH1 inhibitor to the subject; administering or continuing to administer the therapeutic agent that treats or inhibits psoriasis in an amount that is the same as or less than a standard dosage amount to a subject that is heterozygous for the IFIH1 missense variant nucleic acid molecule, and/or administering an IFIH1 inhibitor to the subject; or administering or continuing to administer the therapeutic agent that treats or inhibits psoriasis in an amount that is the same as or less than a standard dosage amount to a subject that is homozygous for the IFIH1 missense variant nucleic acid molecule; wherein the presence of a genotype having the IFIH1 missense variant nucleic acid molecule encoding the IFIH1 predicted loss-of-function polypeptide indicates the subject has a decreased risk of developing psoriasis.

The present disclosure also provides methods of identifying a subject having an increased risk for developing psoriasis, the methods comprising: determining or having determined the presence or absence of an IFIH1 missense variant nucleic acid molecule encoding an IFIH1 predicted loss-of-function polypeptide in a biological sample obtained from the subject; wherein: when the subject is IFIH1 reference, then the subject has an increased risk for developing psoriasis; and when the subject is heterozygous or homozygous for the IFIH1 missense variant nucleic acid molecule encoding an IFIH1 predicted loss-of-function polypeptide, then the subject has a decreased risk for developing psoriasis.

The present disclosure also provides therapeutic agents that treat or inhibit psoriasis for use in the treatment of psoriasis in a subject having a genomic nucleic acid molecule having a nucleotide sequence encoding an IFIH1 polypeptide, wherein the nucleotide sequence comprises a cytosine at a position corresponding to position 38,690 according to SEQ ID NO:2, or the complement thereof.

The present disclosure also provides IFIH1 inhibitors for use in the treatment of psoriasis in a subject that is IFIH1 reference or that is heterozygous for an IFIH1 missense variant nucleic acid molecule encoding an IFIH1 predicted loss-of-function polypeptide.

The present disclosure also provides methods of treating a subject having psoriasis, the methods comprising administering a TRIM65 inhibitor to the subject.

The present disclosure also provides methods of treating a subject with a therapeutic agent that treats or inhibits psoriasis, wherein the subject has psoriasis, the methods comprising the steps of: determining whether the subject has a TRIM65 missense variant nucleic acid molecule encoding a TRIM65 predicted loss-of-function polypeptide by: obtaining or having obtained a biological sample from the subject; and performing or having performed a sequence analysis on the biological sample to determine if the subject has a genotype comprising the TRIM65 missense variant nucleic acid molecule; and administering or continuing to administer the therapeutic agent that treats or inhibits psoriasis in a standard dosage amount to a subject that is TRIM65 reference, and/or administering a TRIM65 inhibitor to the subject; administering or continuing to administer the therapeutic agent that treats or inhibits psoriasis in an amount that is the same as or less than a standard dosage amount to a subject that is heterozygous for the TRIM65 missense variant nucleic acid molecule, and/or administering an TRIM65 inhibitor to the subject; or administering or continuing to administer the therapeutic agent that treats or inhibits psoriasis in an amount that is the same as or less than a standard dosage amount to a subject that is homozygous for the TRIM65 missense variant nucleic acid molecule; wherein the presence of a genotype having the TRIM65 missense variant nucleic acid molecule encoding the TRIM65 predicted loss-of-function polypeptide indicates the subject has a decreased risk of developing psoriasis.

The present disclosure also provides methods of identifying a subject having an increased risk for developing psoriasis, the methods comprising: determining or having determined the presence or absence of a TRIM65 missense variant nucleic acid molecule encoding a TRIM65 predicted loss-of-function polypeptide in a biological sample obtained from the subject; wherein: when the subject is TRIM65 reference, then the subject has an increased risk for developing psoriasis; and when the subject is heterozygous or homozygous for the TRIM65 missense variant nucleic acid molecule encoding a TRIM65 predicted loss-of-function polypeptide, then the subject has a decreased risk for developing psoriasis.

The present disclosure also provides therapeutic agents that treat or inhibit psoriasis for use in the treatment of psoriasis in a subject having a TRIM65 missense variant nucleic acid molecule encoding a TRIM65 predicted loss-of-function polypeptide.

The present disclosure also provides TRIM65 inhibitors for use in the treatment of psoriasis in a subject that is TRIM65 reference or that is heterozygous for a TRIM65 missense variant nucleic acid molecule encoding a TRIM65 predicted loss-of-function polypeptide.

### BRIEF DESCRIPTION OF THE DRAWINGS

The accompanying figures, which are incorporated in and constitute a part of this specification, illustrate several features of the present disclosure.

The patent or application file contains at least one drawing executed in color. Copies of this patent or patent application publication with color drawing(s) will be provided by the Office upon request and payment of the necessary fee.

FIG. 1 shows common TRIM65 psoriasis variant is an eQTL for increased TRIM65 expression in skin.

FIG. 2 shows immunofluorescence analysis of HEK293-HZ cells transiently transfected with N-HA-IFIH1 and N-FlagTRIM65-WT (top) or N-FlagTRIM65-G382R (bottom) which reveals aberrant expression of TRIM65-G382R protein (Panel A); N-HA-IFIH1% colocalization with WT vs TRIM65-G382R (Panel B); % colocalization of WT TRIM65 and TRIM65-G382R with IFIH1 (Panel C); Manders Overlap Coefficient (ranges from 0.0 for no colocalization to 1.0 for full colocalization) between N-HA-IFIH1 and N-FlagTRIM65-WT or N-FlagTRIM65-G382R, which reveals significantly reduced colocalization between IFIH1 and TRIM65-G382R (Panel D); Western blot analysis, which reveals reduced expression of N-Flag-TRIM65-G382R compared to N-Flag-TRIM65-WT (Panel E); and HEK-ISRE-Luc reporter cells transfected with empty vector, N-eGFP-TRIM65-WT, or N-eGFP-TRIM65-G382R (Panels F and G)—ISRE activation was measured by luciferase reporter activity in a 96-well plate in response to 0.05 μg poly(i:c)(Panel F); and 5000 U IFN-α (Panel G); N-eGFP-TRIM65-G382R-transfected cells have reduced ISRE activation compared to N-eGFP-TRIM65-WT in response to poly(i:c) and IFN-α.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by +10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or Alternately phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates. In some embodiments, the subject is a human. In some embodiments, the human is a patient under the care of a physician.

A rare variant in the IFIH1 gene associated with a decreased risk of developing psoriasis in humans has been identified in accordance with the present disclosure. For example, a genetic alteration that changes the guanine of position 38,690 in the human IFIH1 reference (see, SEQ ID NO:1) to cytosine has been observed to indicate that the human having such an alteration may have a decreased risk of developing psoriasis. Altogether, the genetic analyses described herein surprisingly indicate that the IFIH1 gene and, in particular, a variant in the IFIH1 gene, associates with a decreased risk of developing psoriasis. Therefore, subjects that are IFIH1 reference that have an increased risk of developing psoriasis may be treated such that psoriasis is prevented, the symptoms thereof are reduced, and/or development of symptoms is repressed. Accordingly, the present disclosure provides methods of leveraging the identification of such variants in subjects to identify or stratify risk in such subjects of developing psoriasis, such as plaque psoriasis, guttate psoriasis, inverse psoriasis, pustular psoriasis and/or erythrodermic psoriasis, or to diagnose subjects as having an increased risk of developing psoriasis, such as plaque psoriasis, guttate psoriasis, inverse psoriasis, pustular psoriasis and/or erythrodermic psoriasis, such that subjects at risk or subjects with active disease may be treated accordingly.

A TRIM65 Gly382Arg variant has been observed to alter the cellular localization and expression levels of TRIM65. TRIM65 Gly382Arg displays reduced colocalization with its binding partner IFIH1. This observation, along with reduced interferon-stimulated response element (ISRE) activity in response to stimulation by IFN-α or poly(i:c) in cells transfected with a TRIM65 Gly382Arg construct, suggest that this variant likely leads to reduced interferon pathway activation, which may in turn be protective against psoriasis.

It has been further observed in accordance with the present disclosure that an aggregate burden of variations in IFIH1 associate with a lower risk of developing psoriasis. It has been also observed in accordance with the present disclosure that an aggregate burden of IFIH1 missense variant nucleic acid molecules encoding IFIH1 predicted loss-of-function polypeptides have a cumulative protective effect in reducing the risk of developing psoriasis, such as plaque psoriasis, guttate psoriasis, inverse psoriasis, pustular psoriasis and/or erythrodermic psoriasis. Therefore, it is believed that humans having psoriasis may be treated with molecules that inhibit IFIH1. Accordingly, the present disclosure provides methods for leveraging the identification of such variants, and an aggregation burden of having such variants, in subjects to identify or stratify risk in such subjects of psoriasis, or to diagnose subjects as having psoriasis, such that subjects at risk or subjects with active disease may be treated.

For purposes of the present disclosure, any particular subject can be categorized as having one of three IFIH1 genotypes: i) IFIH1 reference; ii) heterozygous for an IFIH1 missense variant nucleic acid molecule encoding IFIH1 predicted loss-of-function polypeptide; or iii) homozygous for an IFIH1 missense variant nucleic acid molecule encoding IFIH1 predicted loss-of-function polypeptide. A subject is IFIH1 reference when the subject does not have a copy of an IFIH1 missense variant nucleic acid molecule encoding IFIH1 predicted loss-of-function polypeptide. A subject is heterozygous for an IFIH1 missense variant nucleic acid molecule encoding IFIH1 predicted loss-of-function polypeptide when the subject has a single copy of an IFIH1 missense variant nucleic acid molecule encoding IFIH1 predicted loss-of-function polypeptide. An IFIH1 missense variant nucleic acid molecule encoding IFIH1 predicted loss-of-function polypeptide is any IFIH1 nucleic acid molecule (such as, a genomic nucleic acid molecule, an mRNA molecule, or a cDNA molecule) encoding an IFIH1 polypeptide having a partial loss-of-function, a complete loss-of-function, a predicted partial loss-of-function, or a predicted complete loss-of-function. A subject who has an IFIH1 polypeptide having a partial loss-of-function (or predicted partial loss-of-function) is hypomorphic for IFIH1. In some embodiments, the IFIH1 missense variant nucleic acid molecule encoding IFIH1 predicted loss-of-function polypeptide is an IFIH1 genomic nucleic acid molecule comprising a cytosine at a position corresponding to position 38,690 according to SEQ ID NO:2. A subject is homozygous for an IFIH1 missense variant nucleic acid molecule encoding IFIH1 predicted loss-of-function polypeptide when the subject has two copies of an IFIH1 missense variant nucleic acid molecule encoding IFIH1 predicted loss-of-function polypeptide.

For subjects that are genotyped or determined to be IFIH1 reference, such subjects have an increased risk of developing psoriasis, such as plaque psoriasis, guttate psoriasis, inverse psoriasis, pustular psoriasis, and/or erythrodermic psoriasis. For subjects that are genotyped or determined to be either IFIH1 reference or heterozygous for an IFIH1 missense variant nucleic acid molecule encoding IFIH1 predicted loss-of-function polypeptide, such subjects can be treated with an IFIH1 inhibitor.

In any of the embodiments described herein, the IFIH1 missense variant nucleic acid molecule encoding IFIH1 predicted loss-of-function polypeptide can be any IFIH1 nucleic acid molecule (such as, for example, genomic nucleic acid molecule, mRNA molecule, or cDNA molecule) encoding an IFIH1 polypeptide having a partial loss-of-function, a complete loss-of-function, a predicted partial loss-of-function, or a predicted complete loss-of-function. For example, the IFIH1 missense variant nucleic acid molecule encoding IFIH1 predicted loss-of-function polypeptide comprises a cytosine at a position corresponding to position 38,690 according to SEQ ID NO:2.

In any of the embodiments described herein, the IFIH1 predicted loss-of-function polypeptide can be any IFIH1 polypeptide having a partial loss-of-function, a complete loss-of-function, a predicted partial loss-of-function, or a predicted complete loss-of-function. In any of the embodiments described herein, the IFIH1 predicted loss-of-function polypeptide can be any of the IFIH1 polypeptides described herein.

For purposes of the present disclosure, any particular subject can be categorized as having one of three TRIM65 genotypes: i) TRIM65 reference; ii) heterozygous for a TRIM65 missense variant nucleic acid molecule encoding a TRIM65 predicted loss-of-function polypeptide; or iii) homozygous for a TRIM65 missense variant nucleic acid molecule encoding a TRIM65 predicted loss-of-function polypeptide. A subject is TRIM65 reference when the subject does not have a copy of a TRIM65 missense variant nucleic acid molecule encoding a TRIM65 predicted loss-of-function polypeptide. A subject is heterozygous for a TRIM65 missense variant nucleic acid molecule encoding a TRIM65 predicted loss-of-function polypeptide when the subject has a single copy of a TRIM65 missense variant nucleic acid molecule encoding a TRIM65 predicted loss-of-function polypeptide. A TRIM65 missense variant nucleic acid molecule encoding A TRIM65 predicted loss-of-function polypeptide is any TRIM65 nucleic acid molecule (such as, a genomic nucleic acid molecule, an mRNA molecule, or a cDNA molecule) encoding a TRIM65 polypeptide having a partial loss-of-function, a complete loss-of-function, a predicted partial loss-of-function, or a predicted complete loss-of-function. A subject who has a TRIM65 polypeptide having a partial loss-of-function (or predicted partial loss-of-function) is hypomorphic for TRIM65. In some embodiments, the TRIM65 predicted loss-of-function polypeptide is TRIM65 Gly382Arg. A subject is homozygous for a TRIM65 missense variant nucleic acid molecule encoding a TRIM65 predicted loss-of-function polypeptide when the subject has two copies of a TRIM65 missense variant nucleic acid molecule encoding a TRIM65 predicted loss-of-function polypeptide.

For subjects that are genotyped or determined to be TRIM65 reference, such subjects have an increased risk of developing psoriasis, such as plaque psoriasis, guttate psoriasis, inverse psoriasis, pustular psoriasis, and/or erythrodermic psoriasis. For subjects that are genotyped or determined to be either TRIM65 reference or heterozygous for a TRIM65 missense variant nucleic acid molecule encoding a TRIM65 predicted loss-of-function polypeptide, such subjects can be treated with a TRIM65 inhibitor.

In any of the embodiments described herein, the TRIM65 missense variant nucleic acid molecule encoding a TRIM65 predicted loss-of-function polypeptide can be any TRIM65 nucleic acid molecule (such as, for example, genomic nucleic acid molecule, mRNA molecule, or cDNA molecule) encoding a TRIM65 polypeptide having a partial loss-of-function, a complete loss-of-function, a predicted partial loss-of-function, or a predicted complete loss-of-function. For example, the TRIM65 missense variant nucleic acid molecule encoding a TRIM65 predicted loss-of-function polypeptide can encode TRIM65 Gly382Arg.

In any of the embodiments described herein, the TRIM65 predicted loss-of-function polypeptide can be any TRIM65 polypeptide having a partial loss-of-function, a complete loss-of-function, a predicted partial loss-of-function, or a predicted complete loss-of-function. In any of the embodiments described herein, the TRIM65 predicted loss-of-function polypeptide can be any of the TRIM65 polypeptides described herein.

In any of the embodiments described herein, psoriasis is plaque psoriasis, guttate psoriasis, inverse psoriasis, pustular psoriasis, or erythrodermic psoriasis, or any combination thereof. In any of the embodiments described herein, the psoriasis is guttate psoriasis. In any of the embodiments described herein, the psoriasis is plaque psoriasis. In any of the embodiments described herein, the psoriasis is inverse psoriasis. In any of the embodiments described herein, the psoriasis is pustular psoriasis. In any of the embodiments described herein, the psoriasis is erythrodermic psoriasis.

Symptoms of plaque psoriasis include raised, inflamed, red lesions on the skin covered with scaly, silvery plaques, typically occurring on elbows, knees, scalp, back, and lower back. Plaque psoriasis is also found on nails, legs, hands, genitals, and breasts. Symptoms of guttate psoriasis include small, pink, individual spots, typically occurring on torso, arms, and legs. Symptoms of inverse psoriasis include bright red lesions that are smooth and shiny, typically occurring on armpits, groin, under the breasts, and skin folds around the genitals and buttocks. Symptoms of pustular psoriasis include yellowish blisters of noninfectious pus surrounded by red skin, either localized to certain areas, such as hands and feet, or covering the body. Symptoms of erythrodermic psoriasis include widespread, fiery redness of the skin and shedding of scales in sheet typically covering most of the body surface.

The present disclosure provides methods of treating a subject having psoriasis, the methods comprising administering an IFIH1 inhibitor to the subject.

The present disclosure also provides methods of treating a subject having guttate psoriasis, the methods comprising administering an IFIH1 inhibitor to the subject.

The present disclosure also provides methods of treating a subject having plaque psoriasis, the methods comprising administering an IFIH1 inhibitor to the subject.

The present disclosure also provides methods of treating a subject having inverse psoriasis, the methods comprising administering an IFIH1 inhibitor to the subject.

The present disclosure also provides methods of treating a subject having pustular psoriasis, the methods comprising administering an IFIH1 inhibitor to the subject.

The present disclosure also provides methods of treating a subject having erythrodermic psoriasis, the methods comprising administering an IFIH1 inhibitor to the subject.

In some embodiments, the IFIH1 inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of an IFIH1 nucleic acid molecule. In some embodiments, the antisense RNA, siRNA, or shRNA hybridizes to a sequence within an IFIH1 genomic nucleic acid molecule or mRNA molecule and decreases expression of the IFIH1 polypeptide in a cell in the subject. In some embodiments, the IFIH1 inhibitor comprises an antisense molecule that hybridizes to an IFIH1 genomic nucleic acid molecule or mRNA molecule and decreases expression of the IFIH1 polypeptide in a cell in the subject. In some embodiments, the IFIH1 inhibitor comprises an siRNA that hybridizes to an IFIH1 genomic nucleic acid molecule or mRNA molecule and decreases expression of the IFIH1 polypeptide in a cell in the subject. In some embodiments, the IFIH1 inhibitor comprises an shRNA that hybridizes to an IFIH1 genomic nucleic acid molecule or mRNA molecule and decreases expression of the IFIH1 polypeptide in a cell in the subject.

The present disclosure also provides methods of treating a subject having psoriasis, the method comprising administering a TRIM65 inhibitor to the subject. In some embodiments, the psoriasis is guttate psoriasis, plaque psoriasis, inverse psoriasis, pustular psoriasis, or erythrodermic psoriasis. In some embodiments, the psoriasis is guttate psoriasis. In some embodiments, the psoriasis is plaque psoriasis. In some embodiments, the psoriasis is inverse psoriasis. In some embodiments, the psoriasis is pustular psoriasis. In some embodiments, the psoriasis is erythrodermic psoriasis.

In some embodiments, the TRIM65 inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of a TRIM65 nucleic acid molecule. In some embodiments, the antisense RNA, siRNA, or shRNA hybridizes to a sequence within a TRIM65 genomic nucleic acid molecule or mRNA molecule and decreases expression of the TRIM65 polypeptide in a cell in the subject. In some embodiments, the TRIM65 inhibitor comprises an antisense molecule that hybridizes to a TRIM65 genomic nucleic acid molecule or mRNA molecule and decreases expression of the TRIM65 polypeptide in a cell in the subject. In some embodiments, the TRIM65 inhibitor comprises an siRNA that hybridizes to a TRIM65 genomic nucleic acid molecule or mRNA molecule and decreases expression of the TRIM65 polypeptide in a cell in the subject. In some embodiments, the TRIM65 inhibitor comprises an shRNA that hybridizes to a TRIM65 genomic nucleic acid molecule or mRNA molecule and decreases expression of the TRIM65 polypeptide in a cell in the subject.

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O—, S—, or N-alkyl; O—, S—, or N-alkenyl; O—, S- or N-alkynyl; or O-alkyl-O-alkyl, wherein the alkyl, alkenyl, and alkynyl may be substituted or unsubstituted C₁₋₁₀ alkyl or C₂₋₁₀ alkenyl, and C₂₋₁₀ alkynyl. Exemplary 2′ sugar modifications also include, but are not limited to, —O[(CH₂)ₙO]ₘCH₃, —O(CH₂)NOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl(2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N₆-methyladenosine, inosine, and N₇-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:

Sense:mN*mN*/i2FN/mN/i2FN/mN/i2FN/mN/i2FN/mN/i2FN/mN/i2FN/mN/i2FN/mN/i2FN/*mN*/32FN/Antisense:/52FN/*/i2FN/*mN/i2FN/mN/i2FN/mN/i2FN/mN/i2FN/mN/i2FN/mN/i2FN/mN/i2FN/mN/i2FN/mN*N*N
wherein: “N” is the base; “2F” is a 2′-F modification; “m” is a 2′-O-methyl modification, “I” is an internal base; and “*” is a phosphorothioate backbone linkage.

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly(lactic acid)(PLA) microspheres, poly(D,L-lactic-coglycolic-acid)(PLGA) microspheres, liposomes, micelles, inverse micelles, lipid cochleates, and lipid microtubules. A carrier may comprise a buffered salt solution such as PBS, HBSS, etc.

In some embodiments, the IFIH1 inhibitor or TRIM65 inhibitor comprises a nuclease agent that induces one or more nicks or double-strand breaks at a recognition sequence(s) or a DNA-binding protein that binds to a recognition sequence within an IFIH1 genomic nucleic acid molecule or a or TRIM65 genomic nucleic acid molecule. The recognition sequence can be located within a coding region of the IFIH1 gene or TRIM65 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the IFIH1 gene or TRIM65 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify an IFIH1 or TRIM65 genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of IFIH1 or TRIM65 nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in an IFIH1 or TRIM65 genomic nucleic acid molecule or it can be a nickase that creates a single-strand break in an IFIH1 or TRIM65 genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of IFIH1 or TRIM65 genomic nucleic acid molecules can be generated by contacting a cell with a Cas protein and one or more gRNAs that hybridize to one or more gRNA recognition sequences within a target genomic locus in the IFIH1 or TRIM65 genomic nucleic acid molecule. For example, a gRNA recognition sequence can be located within a region of SEQ ID NO:1. The gRNA recognition sequence can also include or be proximate to a position corresponding to position 38,690 according to SEQ ID NO:1. For example, the gRNA recognition sequence can be located from about 1000, from about 500, from about 400, from about 300, from about 200, from about 100, from about 50, from about 45, from about 40, from about 35, from about 30, from about 25, from about 20, from about 15, from about 10, or from about 5 nucleotides of a position corresponding to position 38,690 according to SEQ ID NO:1. The gRNA recognition sequence can include or be proximate to the start codon of an IFIH1 or TRIM65 genomic nucleic acid molecule or the stop codon of an IFIH1 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in an IFIH1 or TRIM65 genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where Nis any DNA nucleotide and is immediately 3′ of the gRNA recognition sequence of the non-complementary strand of the target DNA. As such, the PAM sequence of the complementary strand would be 5′-CCN-31, where N is any DNA nucleotide and is immediately 5′ of the gRNA recognition sequence of the complementary strand of the target DNA.

A gRNA is an RNA molecule that binds to a Cas protein and targets the Cas protein to a specific location within an IFIH1 or TRIM65 genomic nucleic acid molecule. An exemplary gRNA is a gRNA effective to direct a Cas enzyme to bind to or cleave an IFIH1 or TRIM65 genomic nucleic acid molecule, wherein the gRNA comprises a DNA-targeting segment that hybridizes to a gRNA recognition sequence within the IFIH1 or TRIM65 genomic nucleic acid molecule that includes or is proximate to a position corresponding to position 38,690 according to SEQ ID NO:1 (for IFIH1). For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of a position corresponding to position 38,690 according to SEQ ID NO:1. Other exemplary gRNAs comprise a DNA-targeting segment that hybridizes to a gRNA recognition sequence present within an IFIH1 or TRIM65 genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

Examples of suitable gRNA recognition sequences located within the human IFIH1 reference gene are set forth in Table 1 as SEQ ID NOs: 24-34.

TABLE 1
Guide RNA Recognition Sequences Near
IFIH1 Variation(s)

| Strand | gRNA Recognition Sequence | SEQ ID NO: |
|---|---|---|
| − | GCAATGGCAAACTTCTTGCATGG | 24 |
| + | AGATGCAACCAGAGAAGTATGGG | 25 |
| + | CAGATGCAACCAGAGAAGTATGG | 26 |
| + | TACTACATTCAGTAGAAAGATGG | 27 |
| + | TCAACTGAAAAACCAAATACAGG | 28 |
| − | TTCTCTGGTTGCATCTGCAATGG | 29 |
| + | ATTCAGTAGAAAGATGGCAAAGG | 30 |
| − | ACATTAAGCCCATACTTCTCTGG | 31 |
| − | TCTTGCATGGCTCCTGTATTTGG | 32 |
| − | TTTGGTTTTTCAGTTGATCAAGG | 33 |
| − | TTTAAATAATATTTTTCAGATGG | 34 |

The Cas protein and the gRNA form a complex, and the Cas protein cleaves the target IFIH1 or TRIM65 genomic nucleic acid molecule. The Cas protein can cleave the nucleic acid molecule at a site within or outside of the nucleic acid sequence present in the target IFIH1 or TRIM65 genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the IFIH1 or TRIM65 genomic nucleic acid molecule to which a DNA-targeting segment of a gRNA will bind.

Such methods can result, for example, in an IFIH1 or TRIM65 genomic nucleic acid molecule in which a region of the gene is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the IFIH1 or TRIM65 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the methods of treatment further comprise detecting the presence or absence of an IFIH1 missense variant nucleic acid molecule encoding an IFIH1 predicted loss-of-function polypeptide in a biological sample from the subject. As used throughout the present disclosure, an “IFIH1 missense variant nucleic acid molecule encoding an IFIH1 predicted loss-of-function polypeptide” is any IFIH1 nucleic acid molecule (such as, for example, genomic nucleic acid molecule, mRNA molecule, or cDNA molecule) encoding an IFIH1 polypeptide having a partial loss-of-function, a complete loss-of-function, a predicted partial loss-of-function, or a predicted complete loss-of-function.

The present disclosure also provides methods of treating a subject with a therapeutic agent that treats or inhibits psoriasis, wherein the subject has psoriasis. In some embodiments, the methods comprise determining whether the subject has an IFIH1 missense variant nucleic acid molecule encoding an IFIH1 predicted loss-of-function polypeptide by obtaining or having obtained a biological sample from the subject, and performing or having performed a sequence analysis on the biological sample to determine if the subject has a genotype comprising the IFIH1 missense variant nucleic acid molecule encoding an IGIH1 predicted loss-of-function polypeptide. In some embodiments, the methods further comprise administering or continuing to administer the therapeutic agent that treats or inhibits psoriasis in a standard dosage amount to a subject that is IFIH1 reference, and/or administering an IFIH1 inhibitor to the subject. In some embodiments, the methods further comprise administering or continuing to administer the therapeutic agent that treats or inhibits psoriasis in an amount that is the same as or less than a standard dosage amount to a subject that is heterozygous for the IFIH1 missense variant nucleic acid molecule, and/or administering an IFIH1 inhibitor to the subject. In some embodiments, the methods further comprise administering or continuing to administer the therapeutic agent that treats or inhibits psoriasis in an amount that is the same as or less than a standard dosage amount to a subject that is homozygous for the IFIH1 missense variant nucleic acid molecule. The presence of a genotype having the IFIH1 missense variant nucleic acid molecule encoding the IFIH1 predicted loss-of-function polypeptide indicates the subject has a decreased risk of developing psoriasis. In some embodiments, the subject is IFIH1 reference. In some embodiments, the subject is heterozygous for a IFIH1 missense variant nucleic acid molecule encoding an IFIH1 predicted loss-of-function polypeptide.

For subjects that are genotyped or determined to be either IFIH1 reference or heterozygous for an IFIH1 missense variant nucleic acid molecule encoding an IFIH1 predicted loss-of-function polypeptide, such subjects can be treated with an IFIH1 inhibitor, as described herein.

Detecting the presence or absence of an IFIH1 missense variant nucleic acid molecule encoding an IFIH1 predicted loss-of-function polypeptide in a biological sample from a subject and/or determining whether a subject has an IFIH1 missense variant nucleic acid molecule encoding an IFIH1 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, the treatment methods further comprise detecting the presence or absence of an IFIH1 predicted loss-of-function polypeptide in a biological sample from the subject. In some embodiments, when the subject does not have an IFIH1 predicted loss-of-function polypeptide, the subject is also administered a therapeutic agent that treats or inhibits psoriasis in a standard dosage amount. In some embodiments, when the subject has an IFIH1 predicted loss-of-function polypeptide, the subject is also administered a therapeutic agent that treats or inhibits psoriasis in a dosage amount that is the same as or less than a standard dosage amount.

The present disclosure also provides methods of treating a subject with a therapeutic agent that treats or inhibits psoriasis, wherein the subject has psoriasis. In some embodiments, the method comprises determining whether the subject has an IFIH1 predicted loss-of-function polypeptide by obtaining or having obtained a biological sample from the subject, and performing or having performed an assay on the biological sample to determine if the subject has an IFIH1 predicted loss-of-function polypeptide. When the subject does not have an IFIH1 predicted loss-of-function polypeptide, the therapeutic agent that treats or inhibits psoriasis is administered or continued to be administered to the subject in a standard dosage amount, and/or an IFIH1 inhibitor is administered to the subject. When the subject has an IFIH1 predicted loss-of-function polypeptide, the therapeutic agent that treats or inhibits psoriasis is administered or continued to be administered to the subject in an amount that is the same as or less than a standard dosage amount, and/or an IFIH1 inhibitor is administered to the subject. The presence of an IFIH1 predicted loss-of-function polypeptide indicates the subject has a decreased risk of developing psoriasis. In some embodiments, the subject has an IFIH1 predicted loss-of-function polypeptide. In some embodiments, the subject does not have an IFIH1 predicted loss-of-function polypeptide.

Detecting the presence or absence of an IFIH1 predicted loss-of-function polypeptide in a biological sample from a subject and/or determining whether a subject has an IFIH1 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

In some embodiments, the IFIH1 inhibitor is a paramyxovirus V protein. In some embodiments, the IFIH1 inhibitor comprises a small molecule.

In any embodiment where a subject that is genotyped or determined to be either IFIH1 reference or heterozygous for an IFIH1 missense variant nucleic acid molecule encoding an IFIH1 predicted loss-of-function polypeptide, such subject can be treated with an inhibitor of type 1 interferon pathway. In some embodiments, the inhibitor of type 1 interferon pathway is an agonist of Adenosine Deaminase RNA Specific (ADAR). In some embodiments, the ADAR agonist is ADAR protein. In some embodiments, the ADAR agonist is an ADAR agonist antibody. In some embodiments, the inhibitor of type 1 interferon pathway is an inhibitor of TRIM65. In some embodiments, the TRIM65 inhibitor comprises a small molecule. In some embodiments, the inhibitor of type 1 interferon pathway is an inhibitor of DEAD-box polypeptide 58 (“DDX58”)(i.e., RIG-1). DDX58 inhibitors include, but are not limited to, epigallocatechin gallate (EGCG) and BX795 (InvivoGen).

In some embodiments, the methods of treatment further comprise detecting the presence or absence of a TRIM65 missense variant nucleic acid molecule encoding a TRIM65 predicted loss-of-function polypeptide in a biological sample from the subject. As used throughout the present disclosure, a “TRIM65 missense variant nucleic acid molecule encoding a TRIM65 predicted loss-of-function polypeptide” is any TRIM65 nucleic acid molecule (such as, for example, genomic nucleic acid molecule, mRNA molecule, or cDNA molecule) encoding a TRIM65 polypeptide having a partial loss-of-function, a complete loss-of-function, a predicted partial loss-of-function, or a predicted complete loss-of-function.

The present disclosure also provides methods of treating a subject with a therapeutic agent that treats or inhibits psoriasis, wherein the subject has psoriasis. In some embodiments, the methods comprise determining whether the subject has a TRIM65 missense variant nucleic acid molecule encoding a TRIM65 predicted loss-of-function polypeptide by obtaining or having obtained a biological sample from the subject, and performing or having performed a sequence analysis on the biological sample to determine if the subject has a genotype comprising the TRIM65 missense variant nucleic acid molecule encoding a TRIM65 predicted loss-of-function polypeptide. In some embodiments, the methods further comprise administering or continuing to administer the therapeutic agent that treats or inhibits psoriasis in a standard dosage amount to a subject that is TRIM65 reference, and/or administering a TRIM65 inhibitor to the subject. In some embodiments, the methods further comprise administering or continuing to administer the therapeutic agent that treats or inhibits psoriasis in an amount that is the same as or less than a standard dosage amount to a subject that is heterozygous for the TRIM65 missense variant nucleic acid molecule, and/or administering a TRIM65 inhibitor to the subject. In some embodiments, the methods further comprise administering or continuing to administer the therapeutic agent that treats or inhibits psoriasis in an amount that is the same as or less than a standard dosage amount to a subject that is homozygous for the TRIM65 missense variant nucleic acid molecule. The presence of a genotype having the TRIM65 missense variant nucleic acid molecule encoding the TRIM65 predicted loss-of-function polypeptide indicates the subject has a decreased risk of developing psoriasis. In some embodiments, the subject is TRIM65 reference. In some embodiments, the subject is heterozygous for a TRIM65 missense variant nucleic acid molecule encoding a TRIM65 predicted loss-of-function polypeptide.

For subjects that are genotyped or determined to be either TRIM65 reference or heterozygous for a TRIM65 missense variant nucleic acid molecule encoding a TRIM65 predicted loss-of-function polypeptide, such subjects can be treated with a TRIM65 inhibitor, as described herein.

Detecting the presence or absence of a TRIM65 missense variant nucleic acid molecule encoding a TRIM65 predicted loss-of-function polypeptide in a biological sample from a subject and/or determining whether a subject has a TRIM65 missense variant nucleic acid molecule encoding a TRIM65 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, the treatment methods further comprise detecting the presence or absence of a TRIM65 predicted loss-of-function polypeptide in a biological sample from the subject. In some embodiments, when the subject does not have a TRIM65 predicted loss-of-function polypeptide, the subject is also administered a therapeutic agent that treats or inhibits psoriasis in a standard dosage amount. In some embodiments, when the subject has a TRIM65 predicted loss-of-function polypeptide, the subject is also administered a therapeutic agent that treats or inhibits psoriasis in a dosage amount that is the same as or less than a standard dosage amount.

The present disclosure also provides methods of treating a subject with a therapeutic agent that treats or inhibits psoriasis, wherein the subject has psoriasis. In some embodiments, the method comprises determining whether the subject has a TRIM65 predicted loss-of-function polypeptide by obtaining or having obtained a biological sample from the subject, and performing or having performed an assay on the biological sample to determine if the subject has a TRIM65 predicted loss-of-function polypeptide. When the subject does not have a TRIM65 predicted loss-of-function polypeptide, the therapeutic agent that treats or inhibits psoriasis is administered or continued to be administered to the subject in a standard dosage amount, and/or a TRIM65 inhibitor is administered to the subject. When the subject has a TRIM65 predicted loss-of-function polypeptide, the therapeutic agent that treats or inhibits psoriasis is administered or continued to be administered to the subject in an amount that is the same as or less than a standard dosage amount, and/or a TRIM65 inhibitor is administered to the subject. The presence of a TRIM65 predicted loss-of-function polypeptide indicates the subject has a decreased risk of developing psoriasis. In some embodiments, the subject has a TRIM65 predicted loss-of-function polypeptide. In some embodiments, the subject does not have a TRIM65 predicted loss-of-function polypeptide.

Detecting the presence or absence of a TRIM65 predicted loss-of-function polypeptide in a biological sample from a subject and/or determining whether a subject has a TRIM65 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

In some embodiments, the TRIM65 inhibitor is a peptide that blocks the interaction between TRIM65 and IFIH1. In some embodiments, the TRIM65 inhibitor comprises a small molecule.

In any embodiment where a subject that is genotyped or determined to be either TRIM65 reference or heterozygous for a TRIM65 missense variant nucleic acid molecule encoding a TRIM65 predicted loss-of-function polypeptide, such subject can be treated with an inhibitor of type 1 interferon pathway and/or an IFIH1 inhibitor. In some embodiments, the inhibitor of type 1 interferon pathway is an agonist of Adenosine Deaminase RNA Specific (ADAR). In some embodiments, the ADAR agonist is ADAR protein. In some embodiments, the ADAR agonist is an ADAR agonist antibody. In some embodiments, the TRIM65 inhibitor comprises a small molecule. In some embodiments, the inhibitor of type 1 interferon pathway is an inhibitor of DEAD-box polypeptide 58 (“DDX58”)(i.e., RIG-1). DDX58 inhibitors include, but are not limited to, epigallocatechin gallate (EGCG) and BX795 (InvivoGen).

Examples of therapeutic agents that treat or inhibit psoriasis include, but are not limited to, an anthralin (dihydroxyanthralin), azaribine, colchicine, fluorouracil, methotrexate, methoxsalen (8-methoxypsoralen), resorcinol, retinoids (such as retinoic acid), corticosteroids (such as clobetasol propionate, triamcinolone acetonide, and the like), cyclosporin, iodochlorhydroxyquin, salicylic acid, vitamin D, dapsone, somatostatin, sulfur, tars, zinc oxide, hydroxycarbamide, fumarates (such as dimethyl fumarate), and ultra-violet light. In some embodiments, the therapeutic agent that treats or inhibits psoriasis is an anthralin (such as dihydroxyanthralin). In some embodiments, the therapeutic agent that treats or inhibits psoriasis is azaribine. In some embodiments, the therapeutic agent that treats or inhibits psoriasis is colchicine. In some embodiments, the therapeutic agent that treats or inhibits psoriasis is fluorouracil. In some embodiments, the therapeutic agent that treats or inhibits psoriasis is methotrexate. In some embodiments, the therapeutic agent that treats or inhibits psoriasis is methoxsalen (such as 8-methoxypsoralen). In some embodiments, the therapeutic agent that treats or inhibits psoriasis is resorcinol. In some embodiments, the therapeutic agent that treats or inhibits psoriasis is a retinoid (such as retinoic acid). In some embodiments, the therapeutic agent that treats or inhibits psoriasis is a corticosteroid (such as clobetasol propionate or triamcinolone acetonide). In some embodiments, the therapeutic agent that treats or inhibits psoriasis is cyclosporin. In some embodiments, the therapeutic agent that treats or inhibits psoriasis is iodochlorhydroxyquin. In some embodiments, the therapeutic agent that treats or inhibits psoriasis is salicylic acid. In some embodiments, the therapeutic agent that treats or inhibits psoriasis is vitamin D. In some embodiments, the therapeutic agent that treats or inhibits psoriasis is dapsone. In some embodiments, the therapeutic agent that treats or inhibits psoriasis is somatostatin. In some embodiments, the therapeutic agent that treats or inhibits psoriasis is sulfur. In some embodiments, the therapeutic agent that treats or inhibits psoriasis is a tar. In some embodiments, the therapeutic agent that treats or inhibits psoriasis is zinc oxide. In some embodiments, the therapeutic agent that treats or inhibits psoriasis is hydroxycarbamide. In some embodiments, the therapeutic agent that treats or inhibits psoriasis is a fumarate (such as dimethyl fumarate). In some embodiments, the therapeutic agent that treats or inhibits psoriasis is ultra-violet light.

In some embodiments, the dose of the therapeutic agents that treat or inhibit psoriasis can be reduced by about 10%, by about 20%, by about 30%, by about 40%, by about 50%, by about 60%, by about 70%, by about 80%, or by about 90% for subjects that are heterozygous for an IFIH1 or TRIM65 missense variant nucleic acid molecule encoding an IFIH1 or TRIM65 predicted loss-of-function polypeptide (i.e., a lower than the standard dosage amount) compared to subjects that are IFIH1 reference or TRIM65 reference (who may receive a standard dosage amount). In some embodiments, the dose of the therapeutic agents that treat or inhibit psoriasis can be reduced by about 10%, by about 20%, by about 30%, by about 40%, or by about 50%. In addition, the dose of therapeutic agents that treat or inhibit psoriasis in subjects that are heterozygous for an IFIH1 or TRIM65 missense variant nucleic acid molecule encoding an IFIH1 or TRIM65 predicted loss-of-function polypeptide can be administered less frequently compared to subjects that are IFIH1 reference or TRIM65 reference.

Administration of the therapeutic agents that treat or inhibit psoriasis and/or IFIH1 inhibitors/TRIM65 inhibitors/type 1 interferon pathway inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the therapeutic agents that treat or inhibit psoriasis and/or IFIH1 inhibitors/TRIM65 inhibitors/type 1 interferon pathway inhibitors can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in psoriasis, a decrease/reduction in the severity of psoriasis (such as, for example, a reduction or inhibition of development or psoriasis), a decrease/reduction in symptoms and psoriasis-related effects, delaying the onset of symptoms and psoriasis-related effects, reducing the severity of symptoms of psoriasis-related effects, reducing the severity of an acute episode, reducing the number of symptoms and psoriasis-related effects, reducing the latency of symptoms and psoriasis-related effects, an amelioration of symptoms and psoriasis-related effects, reducing secondary symptoms, reducing secondary infections, preventing relapse to psoriasis, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of psoriasis development/progression (such as, for example, a complete or partial avoidance/inhibition or a delay), and an increased survival time of the affected host animal, following administration of a therapeutic protocol. Treatment of psoriasis encompasses the treatment of subjects already diagnosed as having any form of psoriasis at any clinical stage or manifestation, the delay of the onset or evolution or aggravation or deterioration of the symptoms or signs of psoriasis, and/or preventing and/or reducing the severity of psoriasis.

The present disclosure also provides methods of diagnosing psoriasis in a subject, the methods comprising determining the subject's aggregate burden of having a plurality of IFIH1 missense variant nucleic acid molecule encoding an IFIH1 predicted loss-of-function polypeptide. When the subject has a lower aggregate burden, and has one or more symptoms of psoriasis, then the subject is diagnosed as having psoriasis. When the subject has a greater aggregate burden, and does not have one or more symptoms of psoriasis, then the subject is diagnosed as not having psoriasis.

The present disclosure also provides methods of identifying a subject having an increased risk for developing psoriasis. In some embodiments, the method comprises determining or having determined in a biological sample obtained from the subject the presence or absence of an IFIH1 missense variant nucleic acid molecule (such as a genomic nucleic acid molecule, mRNA molecule, and/or cDNA molecule) encoding an IFIH1 predicted loss-of-function polypeptide. When the subject lacks an IFIH1 missense variant nucleic acid molecule encoding an IFIH1 predicted loss-of-function polypeptide (i.e., the subject is genotypically categorized as an IFIH1 reference), then the subject has an increased risk for developing psoriasis. When the subject has an IFIH1 missense variant nucleic acid molecule encoding an IFIH1 predicted loss-of-function polypeptide (i.e., the subject is heterozygous for an IFIH1 missense variant nucleic acid molecule encoding an IFIH1 predicted loss-of-function polypeptide), then the subject has a decreased risk for developing psoriasis.

Having a single copy of an IFIH1 missense variant nucleic acid molecule encoding an IFIH1 predicted loss-of-function polypeptide is more protective of a subject from developing psoriasis than having no copies of an IFIH1 missense variant nucleic acid molecule encoding an IFIH1 predicted loss-of-function polypeptide. Without intending to be limited to any particular theory or mechanism of action, it is believed that a single copy of an IFIH1 missense variant nucleic acid molecule encoding an IFIH1 predicted loss-of-function polypeptide (i.e., heterozygous for an IFIH1 missense variant nucleic acid molecule encoding an IFIH1 predicted loss-of-function polypeptide) is protective of a subject from developing psoriasis, and it is also believed that having two copies of an IFIH1 missense variant nucleic acid molecule encoding an IFIH1 predicted loss-of-function polypeptide (i.e., homozygous for an IFIH1 missense variant nucleic acid molecule encoding an IFIH1 predicted loss-of-function polypeptide) may be more protective of a subject from developing psoriasis, relative to a subject with a single copy. Thus, in some embodiments, a single copy of an IFIH1 missense variant nucleic acid molecule encoding an IFIH1 predicted loss-of-function polypeptide may not be completely protective, but instead, may be partially or incompletely protective of a subject from developing psoriasis. While not desiring to be bound by any particular theory, there may be additional factors or molecules involved in the development of psoriasis that are still present in a subject having a single copy of an IFIH1 missense variant nucleic acid molecule encoding an IFIH1 predicted loss-of-function polypeptide, thus resulting in less than complete protection from the development of psoriasis.

Determining whether a subject has an IFIH1 missense variant nucleic acid molecule encoding an IFIH1 predicted loss-of-function polypeptide in a biological sample from a subject and/or determining whether a subject has an IFIH1 missense variant nucleic acid molecule encoding an IFIH1 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

The present disclosure also provides methods of identifying a subject having an increased risk for developing psoriasis. In some embodiments, the method comprises determining or having determined in a biological sample obtained from the subject the presence or absence of a TRIM65 missense variant nucleic acid molecule (such as a genomic nucleic acid molecule, mRNA molecule, and/or cDNA molecule) encoding a TRIM65 predicted loss-of-function polypeptide. When the subject lacks a TRIM65 missense variant nucleic acid molecule encoding a TRIM65 predicted loss-of-function polypeptide (i.e., the subject is genotypically categorized as TRIM65 reference), then the subject has an increased risk for developing psoriasis. When the subject has a TRIM65 missense variant nucleic acid molecule encoding a TRIM65 predicted loss-of-function polypeptide (i.e., the subject is heterozygous for a TRIM65 missense variant nucleic acid molecule encoding a TRIM65 predicted loss-of-function polypeptide), then the subject has a decreased risk for developing psoriasis.

Having a single copy of a TRIM65 missense variant nucleic acid molecule encoding a TRIM65 predicted loss-of-function polypeptide is more protective of a subject from developing psoriasis than having no copies of a TRIM65 missense variant nucleic acid molecule encoding a TRIM65 predicted loss-of-function polypeptide. Without intending to be limited to any particular theory or mechanism of action, it is believed that a single copy of a TRIM65 missense variant nucleic acid molecule encoding a TRIM65 predicted loss-of-function polypeptide (i.e., heterozygous for a TRIM65 missense variant nucleic acid molecule encoding a TRIM65 predicted loss-of-function polypeptide) is protective of a subject from developing psoriasis, and it is also believed that having two copies of a TRIM65 missense variant nucleic acid molecule encoding a TRIM65 predicted loss-of-function polypeptide (i.e., homozygous for a TRIM65 missense variant nucleic acid molecule encoding a TRIM65 predicted loss-of-function polypeptide) may be more protective of a subject from developing psoriasis, relative to a subject with a single copy. Thus, in some embodiments, a single copy of a TRIM65 missense variant nucleic acid molecule encoding a TRIM65 predicted loss-of-function polypeptide may not be completely protective, but instead, may be partially or incompletely protective of a subject from developing psoriasis. While not desiring to be bound by any particular theory, there may be additional factors or molecules involved in the development of psoriasis that are still present in a subject having a single copy of a TRIM65 missense variant nucleic acid molecule encoding a TRIM65 predicted loss-of-function polypeptide, thus resulting in less than complete protection from the development of psoriasis.

Determining whether a subject has a TRIM65 missense variant nucleic acid molecule encoding a TRIM65 predicted loss-of-function polypeptide in a biological sample from a subject and/or determining whether a subject has a TRIM65 missense variant nucleic acid molecule encoding a TRIM65 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, any of the methods described herein can further comprise determining the subject's aggregate burden of having an IFIH1 missense variant nucleic acid molecule encoding an IFIH1 predicted loss-of-function polypeptide, and/or an IFIH1 predicted loss-of-function variant polypeptide associated with a decreased risk of developing psoriasis. The aggregate burden is the sum of all variants in the IFIH1 gene, which can be carried out in an association analysis with psoriasis. In some embodiments, the subject is homozygous for one or more IFIH1 missense variant nucleic acid molecules encoding an IFIH1 predicted loss-of-function polypeptide associated with a decreased risk of developing psoriasis. In some embodiments, the subject is heterozygous for one or more IFIH1 missense variant nucleic acid molecules encoding an IFIH1 predicted loss-of-function polypeptide associated with a decreased risk of developing psoriasis. The result of the association analysis suggests that IFIH1 missense variant nucleic acid molecules encoding an IFIH1 predicted loss-of-function polypeptide are associated with decreased risk of developing psoriasis. When the subject has a lower aggregate burden, the subject is at a higher risk of developing psoriasis and the subject is administered or continued to be administered the therapeutic agent that treats or inhibits psoriasis in a standard dosage amount. When the subject has a greater aggregate burden, the subject is at a lower risk of developing psoriasis and the subject is administered or continued to be administered the therapeutic agent that treats or inhibits psoriasis in an amount that is the same as or less than the standard dosage amount. The greater the aggregate burden, the lower the risk of developing psoriasis. Table 2 lists exemplary IFIH1 variant nucleic acid molecules that can be used in an aggregate burden analysis.

TABLE 2

| Variant | rsID | Effect | HGVS.c | HGVS.p |
|---|---|---|---|---|
| 2:162279995:C:G | rs35337543 | splice_donor | c.1641 + 1G > C | |
| 2:162268086:C:T | rs35732034 | splice_donor | c.2807 + 1G > A | |
| 2:162277580:C:A | rs35744605 | stop_gained | c.1879G > T | p.Glu627* |
| 2:162278205:C:CT | rs553669430 | frameshift | c.1764dupA | p.Ala589fs |
| 2:162277435:AATCT:A | rs569337014 | frameshift | c.2020_2023delAGAT | p.Arg674fs |
| 2:162277442:CT:C | rs773033563 | frameshift | c.2016delA | p.Asp673fs |
| 2:162267515:A:C | rs750063177 | stop_gained | c.2862T > G | p.Tyr954* |
| 2:162273932:C:A | rs201472224 | stop_gained | c.2317G > T | p.Glu773* |
| 2:162276691:GT:G | rs759430873 | frameshift | c.2299delA | p.Thr767fs |
| 2:162277422:TAA:T | rs774076578 | frameshift | c.2035_2036delTT | p.Leu679fs |
| 2:162277651:TG:T | | frameshift | c.1807delC | p.His603fs |
| 2:162280040:G:A | | stop_gained | c.1597C > T | p.Gln533* |
| 2:162280047:GT:G | rs779192156 | frameshift | c.1589delA | p.Asn530fs |
| 2:162267201:TA:T | | frameshift | c.3076delT | p.Ter1026fs |
| 2:162267202:A:T | | stop_lost | c.3076T > A | p.Ter1026Lyse |
| | | | | xt*? |
| 2:162267213:CTAAA:C | | frameshift | c.3061_3064delTTTA | p.Phe1021fs |
| 2:162267222:CA:C | rsl392296770 | frameshift | c.3055delT | p.Cys1019fs |
| 2:162267270:C:T | | stop_gained | c.3008G > A | p.Trp1003* |
| 2:162267271:AC:A | | frameshift | c.3006delG | p.Lys1002fs |
| 2:162267274:T:A | | stop_gained | c.3004A > T | p.Lys1002* |
| 2:162267278:G:T | rsl51037370 | stop_gained | c.3000C > A | p.Tyr1000* |
| 2:162267304:T:A | | stop_gained | c.2974A > T | p.Lys992* |
| 2:162267324:CTTATT:C | | frameshift | c.2949_2953delAATAA | p.Ile984fs |
| 2:162267335:A:T | | stop_gained | c.2943T > A | p.Cys981* |
| 2:162267336:CA:C | | frameshift | c.2941delT | p.Cys981fs |
| 2:162267348:A:C | | stop_gained | c.2930T > G | p.Leu977* |
| 2:162267375:C:T | | stop_gained | c.2903G > A | p.Trp968* |
| 2:162267381:T:C | | splice_acceptor | c.2899-2A > G | |
| 2:162267478:C:A | | splice_donor | c.2898 + 1G > T | |
| 2:162267485:AC:A | rs761697569 | frameshift | c.2891delG | p.Cys964fs |
| 2:162267491:G:T | rs1423577496 | stop_gained | c.2886C > A | p.Cys962* |
| 2:162267514:G:A | rs144455277 | stop_gained | c.2863C > T | p.Gln955* |
| 2:162267541:CT:C | | frameshift | c.2835delA | p.Ala946fs |
| 2:162267544:TG:T | | frameshift | c.2832delC | p.Asn944fs |
| 2:162267550:C:A | | stop_gained | c.2827G > T | p.Glu943* |
| 2:162267560:G:T | | stop_gained | c.2817C > A | p.Tyr939* |
| 2:162267564:AG:A | | frameshift | c.2812delC | p.Leu938fs |
| 2:162267568:CCCTA:C | | frameshift | c.2808-3_2808delTAGG | p.Glu937fs |
| 2:162267570:C:T | | splice_acceptor | c.2808-1G > A | |
| 2:162268086:C:G | | splice_donor | c.2807 + lG > C | |
| 2:162268164:G:T | rs775467204 | stop_gained | c.2730C > A | p.Cys910* |
| 2:162268192:G:C | | stop_gained | c.2702C > G | p.Ser901* |
| 2:162268206:G:T | rs946205846 | stop_gained | c.2688C > A | p.Tyr896* |
| 2:162268207:TA:T | | frameshift | c.2686delT | p.Tyr896fs |
| 2:162268229:T:A | rsl252022173 | stop_gained | c.2665A > T | p.Lys889* |
| 2:162268254: ACTTTGCAT:A | | frameshift | c.2632_2639delATGCAAAG | p.Met878fs |
| 2:162268259:G:A | | stop_gained | c.2635C > T | p.Gln879* |
| 2:162268263:CT:C | | frameshift | c.2630delA | p.Gln877fs |
| 2:162268265:G:GT | | frameshift | c.2628dupA | p.Gln877fs |
| 2:162268265:G:A | | stop_gained | c.2629C > T | p.Gln877* |
| 2:162268271:C:A | | stop_gained | c.2623G > T | p.Glu875* |
| 2:162268272:CA:C | | frameshift | c.2621delT | p.Leu874fs |
| 2:162268278:C:A | | splice_acceptor | c.2617-1G > T | |
| 2:162272225:C:T | rs1428749026 | splice_donor | c.2616 + 1G > A | |
| 2:162272225:C:A | | splice_donor | c.2616 + 1G > T | |
| 2:162272260:AC:A | | frameshift | c.2581delG | p.Val861fs |
| 2:162272271:AGCTT:A | rs764990040 | frameshift | c.2567_2570delAAGC | p.Lys856fs |
| 2:162272277:A:T | | stop_gained | c.2565T > A | p.Tyr855* |
| 2:162272287:TTCTC:T | | frameshift | c.2551_2554delGAGA | p.Glu851fs |
| 2:162272294:G:A | rs750804689 | stop_gained | c.2548C > T | p.Arg850* |
| 2:162272299:TC:T | | frameshift | c.2542delG | p.Asp848fs |
| 2:162272306:CT:C | | frameshift | c.2535delA | p.Val846fs |
| 2:162272312:C:A | | stop_gained | c.2530G > T | p.Glu844* |
| 2:162272336:T:TGTGA | | frameshift | c.2502_2505dupTCAC | p.Gly837fs |
| 2:162272352:G:T | | stop_gained | c.2490C > A | p.Tyr830* |
| 2:162272378:G:A | rs747926684 | stop_gained | c.2464C > T | p.Arg822* |
| 2:162272389:T:G | | splice_acceptor | c.2455-2A > C | |
| 2:162273793:A:G | | splice_donor | c.2454 + 2T > C | |
| 2:162273793:A:T | | splice_donor | c.2454 + 2T > A | |
| 2:162273794:C:G | | splice_donor | c.2454 + 1G > C | |
| 2:162273794:C:A | rs778780074 | splice_donor | c.2454 + 1G > T | |
| 2:162273801:CA:C | rsl349369420 | frameshift | c.2447delT | p.Met816fs |
| 2:162273806:CTATT:C | | frameshift | c.2439_2442delAATA | p.Ile814fs |
| 2:162273812:C:A | | stop_gained | c.2437G > T | p.Glu813* |
| 2:162273818:TGACG:T | | frameshift | c.2427_2430delCGTC | p.Val810fs |
| 2:162273828:A:C | rsl84259770 | stop_gained | c.2421T > G | p.Tyr807* |
| 2:162273851:C:A | rs1444920926 | stop_gained | c.2398G > T | p.Glu800* |
| 2:162273901:A:AT | rs765769492 | frameshift | c.2347dupA | p.Ile783fs |
| 2:162273901:AT:A | rs751055123 | frameshift | c.2347delA | p.Ile783fs |
| 2:162273931:TC:T | rs746375033 | frameshift | c.2317delG | p.Glu773fs |
| 2:162273946:T:C | rsl99696786 | splice_acceptor | c.2305-2A > G | |
| 2:162276686:C:T | rs762865950 | splice_donor | c.2304 + 1G > A | |
| 2:162276686:C:A | rs762865950 | splice_donor | c.2304 + 1G > T | |
| 2:162276687:C:CT | | frameshift | c.2303dupA | p.Asn769fs |
| 2:162276704:ACT:A | | frameshift | c.2285_2286delAG | p.Glu762fs |
| 2:162276710:TG:T | rs771155568 | frameshift | c.2280delC | p.Ser760fs |
| 2:162276733:TG:T | rs778910754 | frameshift | c.2257delC | p.His753fs |
| 2:162276739:GC:G | | frameshift | c.2251delG | p.Ala751fs |
| 2:162276768:ATTTTCAG:A | rs745948096 | frameshift | c.2216_2222delCTGAAAA | p.Thr739fs |
| 2:162276780:C:T | | stop_gained | c.2211G > A | p.Trp737* |
| 2:162276795:A:T | rs147175706 | stop_gained | c.2196T > A | p.Tyr732* |
| 2:162276795:A:C | | stop_gained | c.2196T > G | p.Tyr732* |
| 2:162276804:CT:C | | frameshift | c.2186delA | p.Gln729fs |
| 2:162276809:GT:G | | frameshift | c.2181delA | p.Arg728fs |
| 2:162276809:G:A | rs201193151 | stop_gained | c.2182C > T | p.Arg728* |
| 2:162276830:C:A | | stop_gained | c.2161G > T | p.Gly721* |
| 2:162276833:G:A | rs761864966 | stop_gained | c.2158C > T | p.Arg720* |
| 2:162276860:G:A | | stop_gained | c.2131C > T | p.Gln711* |
| 2:162276871:G:GT | | frameshift | c.2119dupA | p.Thr707fs |
| 2:162276884:TG:T | | frameshift | c.2106delC | p.Lys703fs |
| 2:162276889:AG:A | | frameshift | c.2101delC | p.Leu701fs |
| 2:162276903:A:T | rs761764756 | stop_gained | c.2088T > A | p.Tyr696* |
| 2:162276910:GGGTTTTCA:G | | frameshift | c.2073_2080delTGAAAACC | p.Glu692fs |
| 2:162276922:AG:A | | frameshift | c.2068delC | p.Leu690fs |
| 2:162276923:GC:G | | frameshift | c.2067delG | p.Arg689fs |
| 2:162276925:CT:C | rsll90347967 | frameshift | c.2065delA | p.Arg689fs |
| 2:162276934:AT:A | | frameshift | c.2056delA | p.Met686fs |
| 2:162276947:C:T | rs748813106 | splice_acceptor | c.2045-1G > A | |
| 2:162276947:C:A | rs748813106 | splice_acceptor | c.2045-1G > T | |
| 2:162277413:AC:A | | splice_donor | c.2044 + 1delG | |
| 2:162277413:A:G | rs201026962 | splice_donor | c.2044 + 2T > C | |
| 2:162277414:C:T | | splice_donor | c.2044 + lG > A | |
| 2:162277415:C:CA | rs1475939758 | frameshift | c.2043dupT | p.Glu682fs |
| 2:162277422:T:TA | | frameshift | c.2036dupT | p.Leu679fs |
| 2:162277438:CTA:C | | frameshift | c.2019_2020delTA | p.Asp673fs |
| 2:162277439:T:A | | stop_gained | c.2020A > T | p.Arg674* |
| 2:162277440:ATC:A | | frameshift | c.2017_2018delGA | p.Asp673fs |
| 2:162277466:T:A | | stop_gained | c.1993A > T | p.Lys665* |
| 2:162277471:A:T | | stop_gained | c.1988T > A | |
| 2:162277515:AC:A | rs751231371 | frameshift | c.1943delG | p.Gly648fs |
| 2:162277535:CTA:C | | frameshift | c.1922_1923delTA | p.Ile641fs |
| 2:162277539:G:GT | | frameshift | c.1919_1920insA | p.Ile641fs |
| 2:162277562:C:A | | stop_gained | c.1897G > T | p.Glu633* |
| 2:162277590:A:T | | stop_gained | c.1869T > A | p.Tyr623* |
| 2:162277607:GA:G | | frameshift | c.1851delT | p.Arg618fs |
| 2:162277607:G:A | rs745937740 | stop_gained | c.1852C > T | p.Arg618* |
| 2:162277612:GT:G | | frameshift | c.1846delA | p.Thr616fs |
| 2:162277661:AAACACG | rs766039450 | frameshift | c.1787_1797delAA | p.Lys596fs |
| I IUI 1:A | | | GAACGTGTT | |
| 2:162277663:AC:A | rs773795297 | frameshift | c.1795delG | p.Val599fs |
| 2:162277664:C:CA | | frameshift | c.1794dupT | p.Val599fs |
| 2:162277665:AC:A | | frameshift | c.1793delG | p.Arg598fs |
| 2:162277670:CT:C | | frameshift | c.1788delA | p.Glu597fs |
| 2:162277682:C:A | | stop_gained | c.1777G > T | p.Gly593* |
| 2:162277685:C:A | rs1025854546 | stop_gained | c.1774G > T | p.Glu592* |
| 2:162277694:C:T | | splice_acceptor | c.1766-1G > A | |
| 2:162277695:T:A | | splice_acceptor | c.1766-2A > T | |
| 2:162278203:A:G | rs995063479 | splice_donor | c.1765 + 2T > C | |
| 2:162278203:AC:A | rsl227440828 | splice_donor | c.1765 + 1delG | |
| 2:162278204:C:T | rsl292220162 | splice_donor | c.1765 + 1G > A | |
| 2:162278205:CT:C | rs753152979 | frameshift | c.1764delA | p.Ala589fs |
| 2:162278227:C:T | rsl370550497 | stop_gained | c.1743G > A | p.Trp581* |
| 2:162278232:G:A | rs774888783 | stop_gained | c.1738C > T | p.Gln580* |
| 2:162278244:G:A | | stop_gained | c.1726C > T | p.Gln576* |
| 2:162278248:TC:T | | frameshift | c.1721delG | p.Gly574fs |
| 2:162278250:CA:C | | frameshift | c.1719delT | p.Phe573fs |
| 2:162278258:G:C | | stop_gained | c.1712C > G | p.Ser571* |
| 2:162278265:GA:G | | frameshift | c.1704delT | p.Pro569fs |
| 2:162278279:TAA:T | rs1464362256 | frameshift | c.1689_1690delTT | p.Tyr564fs |
| 2:162278286:GA:G | | frameshift | c.1683delT | p.Gln562fs |
| 2:162278297:AT:A | | frameshift | c.1672delA | p.Met558fs |
| 2:162278319:TA:T | | frameshift | c.1650delT | p.Phe550fs |
| 2:162278319:T:A | | stop_gained | c.1651A > T | p.Lys551* |
| 2:162278326:AT:A | | frameshift | c.1643delA | p.Asp548fs |
| 2:162278330:T:G | | splice_acceptor | c.1642-2A > C | |
| 2:162278330:T:C | | splice_acceptor | c.1642-2A > G | |
| 2:162279994:A:C | | splice_donor | c.1641 + 2T > G | |
| 2:162279994:A:G | rs747781178 | splice_donor | c.1641 + 2T > C | |
| 2:162279995:C:T | rs35337543 | splice_donor | c.1641 + 1G > A | |
| 2:162279995:C:A | | splice_donor | c.1641 + 1G > T | |
| 2:162280001:TG:T | | frameshift | c.1635delC | p.Arg546fs |
| 2:162280004:TTG:T | | frameshift | c.1631_1632delCA | p.Ala544fs |
| 2:162280011:TG:T | | frameshift | c.1625delC | p.Ala542fs |
| 2:162280016:TG:T | | frameshift | c.1620delC | p.Ile541fs |
| 2:162280047:G:GT | | frameshift | c.1589dupA | p.Asn530fs |
| 2:162280055:G:GT | | frameshift | c.1581dupA | p.Leu528fs |
| 2:162280058:G:A | | stop_gained | c.1579C > T | p.Gln527* |
| 2:162280066:TTTTC:T | | frameshift | c.1567_1570delGAAA | p.Glu523fs |
| 2:162280073:T:A | | stop_gained | c.1564A > T | p.Lys522* |
| 2:162280076:CA:C | | frameshift | c.1560delT | p.Val521fs |
| 2:162280078:GTT:G | rs1466828817 | frameshift | c.1557_1558delAA | p.Lys519fs |
| 2:162280082:T:A | rsll90299758 | stop_gained | c.1555A > T | p.Lys519* |
| 2:162280088:TA:T | | frameshift | c.1548delT | p.Phe516fs |
| 2:162280105:GCA:G | | frameshift | c.1530_1531delTG | p.Ala511fs |
| 2:162280109:A:AGC | | frameshift | c.1527_1528insGC | p.Cys510fs |
| 2:162280113:C:G | | splice_acceptor | c.1525-1G > C | |
| 2:162280113:C:T | rs774035953 | splice_acceptor | c.1525-1G > A | |
| 2:162280114:T:C | | splice_acceptor | c.1525-2A > G | |
| 2:162280114:T:A | | splice_acceptor | c.1525-2A > T | |
| 2:162281326:A:T | rs865898522 | splice_donor | c.1524 + 2T > A | |
| 2:162281326:A:C | rs865898522 | splice_donor | c.1524 + 2T > G | |
| 2:162281327:C:T | rs898118498 | splice_donor | c.1524 + 1G > A | |
| 2:162281327:C:A | rs898118498 | splice_donor | c.1524 + 1G > T | |
| 2:162281331:T:TA | | frameshift | c.1520dupT | p.Leu507fs |
| 2:162281332:A:T | | stop_gained | c.1520T > A | p.Leu507* |
| 2:162281342:C:A | rs772032662 | stop_gained | c.1510G > T | p.Glu504* |
| 2:162281348:CT:C | | frameshift | c.1503delA | p.Ala502fs |
| 2:162281352:GGCTT:G | rs758450641 | frameshift | c.1496_1499delAAGC | p.Gln499fs |
| 2:162281361:CG:C | rs781305209 | frameshift | c.1490delC | p.Thr497fs |
| 2:162281365:GCCCCTCCA:G | | frameshift | c.1479_1486delTGGAGGGG | p.Gly494fs |
| 2:162281375:C:CT | | frameshift | c.1476_1477insA | p.Va1493fs |
| 2:162281401:A:AAGGGGAT | rs748061170 | frameshift | c.1450_1451insATCCCCT | p.Ile484fs |
| 2:162281405:G:A | rs763638412 | stop_gained | c.1447C > T | p.Gln483* |
| 2:162281426:T:A | rs778487639 | stop_gained | c.1426A > T | p.Lys476* |
| 2:162281432:CT:C | | frameshift | c.1419delA | p.Glu474fs |
| 2:162281434:1ILIIGAGTCTA:T | | frameshift | c.1407_1417delTAGACTCAAGA | p.Asn469fs |
| 2:162281436:CTT:C | | frameshift | c.1414_1415delAA | p.Lys472fs |
| 2:162281445:AT:A | | frameshift | c.1406delA | p.Asn469fs |
| 2:162281453:TC:T | | frameshift | c.1398delG | p.Asn468fs |
| 2:162281467:A:T | rs779927507 | stop_gained | c.1385T > A | p.Leu462* |
| 2:162281488:T:TTA | | frameshift | c.1362_1363dupTA | p.Asn455fs |
| 2:162281490:A:C | | stop_gained | c.1362T > G | p.Tyr454* |
| 2:162281498:CT:C | | frameshift | c.1353delA | p.Ala452fs |
| 2:162281501:CT:C | | frameshift | c.1350delA | p.Glu451fs |
| 2:162281508:GGT:G | rsll95101187 | frameshift | c.1342_1343delAC | p.Thr448fs |
| 2:162281519:AT:A | rs773197026 | frameshift | c.1332delA | p.Glu444fs |
| 2:162281539:GAA:G | | frameshift | c.1311_1312delTT | p.Ser438fs |
| 2:162281546:C:A | rs779764925 | splice_acceptor | c.1307-1G > T | |
| 2:162282365:C:T | | splice_donor | c.1306 + 1G > A | |
| 2:162282365:C:A | rs1002771119 | splice_donor | c.1306 + 1G > T | |
| 2:162282399:C:A | rs1054159671 | stop_gained | c.1273G > T | p.Glu425* |
| 2:162282400:CA:C | rsll80019447 | frameshift | c.1271delT | p.Leu424fs |
| 2:162282468:C:A | | stop_gained | c.1204G > T | p.Glu402* |
| 2:162282514:AC:A | | frameshift | c.H57delG | p.Arg386fs |
| 2:162282517:A:T | | stop_gained | c.H55T > A | p.Tyr385* |
| 2:162282520:C:T | rsl99917968 | stop_gained | c.1152G > A | p.Trp384* |
| 2:162282521:C:T | rs752544919 | stop_gained | c.H51G > A | p.Trp384* |
| 2:162282561:G:A | rsl285685926 | stop_gained | c.1111C > T | p.Gln371* |
| 2:162282570:GC:G | | frameshift | c.H01delG | p.Leu368fs |
| 2:162282577:CT:C | | splice_acceptor | c.1096-2delA | |
| 2:162282577:C:T | | splice_acceptor | c.1096-1G > A | |
| 2:162288134:C:T | rs140125523 | splice_donor | c.1095 + 1G > A | |
| 2:162288134:C:A | rs140125523 | splice_donor | c.1095 + 1G > T | |
| 2:162288154:AC:A | | frameshift | c.1075delG | p.Val359fs |
| 2:162288167:C:A | | stop_gained | c.1063G > T | p.Glu355* |
| 2:162288183:CTTGT:C | | frameshift | c.1043_1046delACAA | p.Asp348fs |
| 2:162288189:TA:T | | frameshift | c.1040delT | p.Leu347fs |
| 2:162288236:CT:C | | frameshift | c.993delA | p.Ser333fs |
| 2:162288281:G:A | rs74162079 | stop_gained | c.949C > T | p.Gln317* |
| 2:162288292:A:AC | | frameshift | c.937_938insG | p.Met313fs |
| 2:162288292:AT:A | rs757577285 | frameshift | c.937delA | p.Met313fs |
| 2:162288297:G:C | | stop_gained | c.933C > G | p.Tyr311* |
| 2:162288297:G:T | rs1057520076 | stop_gained | c.933C > A | p.Tyr311* |
| 2:162288300:AG:A | rsl212715492 | frameshift | c.929delC | p.Pro310fs |
| 2:162288311:G:A | rs762821474 | stop_gained | c.919C > T | p.Gln307* |
| 2:162288324:CG:C | | frameshift | c.905delC | p.Pro302fs |
| 2:162288342:CA:C | | frameshift | c.887delT | p.Val296fs |
| 2:162288343:AC:A | rsl259622346 | frameshift | c.886delG | p.Val296fs |
| 2:162288356:C:G | | splice_acceptor | c.875-1G > C | |
| 2:162293562:ACCTGAA | rsll95353990 | frameshift | c.855_874 + IdelCA | p.Thr286fs |
| TCACTTCCCATGGTG:A | | | CCATGGGAAGTGATTCAGG | |
| 2:162293562:A:G | | splice_donor | c.874 + 2T > C | |
| 2:162293563:C:T | | splice_donor | c.874 + 1G > A | |
| 2:162293583:G:GCCTGAAT | | frameshift | c.848_854dupATTCAGG | p.Thr286fs |
| 2:162293643:TC:T | rs758585876 | frameshift | c.794delG | p.Gly265fs |
| 2:162293669:C:T | | splice_acceptor | c.770-1G > A | |
| 2:162306706:T:TACCTG | rsl323841528 | splice_donor | c.769 + 2_769 + 3insCAGGT | |
| 2:162306708:C:A | | splice_donor | c.769 + 1G > T | |
| 2:162306742:C:A | rs373854773 | stop_gained | c.736G > T | p.Glu246* |
| 2:162306744:G:T | | stop_gained | c.734C > A | p.Ser245* |
| 2:162306747:GA:G | | frameshift | c.730delT | p.Ser244fs |
| 2:162306761:G:GC | | frameshift | c.716dupG | p.Met240fs |
| 2:162306790:G:A | rs771251917 | stop_gained | c.688C > T | p.Gln230* |
| 2:162306811:G:A | rs1463635016 | stop_gained | c.667C > T | p.Gln223* |
| 2:162306823:G:A | | stop_gained | c.655C > T | p.Gln219* |
| 2:162306851:AAT:A | | frameshift | c.625_626delAT | p.Ile209fs |
| 2:162310723:AG:A | | frameshift | c.663delC | p.Ter222fs |
| 2:162310723:A:T | | stop_lost | c.664T > A | p.Ter222Lysex |
| | | | | t*? |
| 2:162310733:TG:T | | frameshift | c.653delC | p.Ser218fs |
| 2:162310741:CT:C | | frameshift | c.645delA | p.Asp216fs |
| 2:162310750:TA:T | | frameshift | c.636delT | p.Phe212fs |
| 2:162310765:C:A | | stop_gained | c.622G > T | p.Glu208* |
| 2:162310777:C:A | | stop_gained | c.610G > T:c.610G > T | p.Glu204*:p.GI |
| | | | | u204* |
| 2:162310822:C:A | | stop_gained | c.565G > T:c.565G > T | p.Glyl89*:p.GI |
| | | | | yl89* |
| 2:162310855:AC:A | | frameshift | c.531delG:c.531delG | p.Trp177fs:p.T |
| | | | | rp177fs |
| 2:162310857:C:T | | stop_gained | c.530G > A:c.530G > A | p.Trp177*:p.Tr |
| | | | | p177* |
| 2:162310868:CT:C | rsl348521704 | frameshift | c.518delA:c.518delA | p.Gln173fs:p.G |
| | | | | In173fs |
| 2:162310878:CT:C | | frameshift | c.508delA:c.508delA | p.Arg170fs:p.A |
| | | | | rg170fs |
| 2:162310882:T:A | rs201495678 | stop_gained | c.505A > T:c.505A > T | p.Lys169*:p.Ly |
| | | | | s169* |
| 2:162310891:C:A | | stop_gained | c.496G > T:c.496G > T | p.Glul66*:p.GI |
| | | | | ul66* |
| 2:162310902:G:T | rs1455983191 | stop_gained | c.485C > A:c.485C > A | p.Ser162*:p.Se |
| | | | | r162* |
| 2:162310903:A:AT | | frameshift | c.483dupA:c.483du | p.Ser162fs:p.S |
| | | | pA | er162fs |
| 2:162310906:C:A | | stop_gained | c.481G > T:c.481G > T | p.Glu161*:p.GI |
| | | | | U161* |
| 2:162310924:CA:C | | frameshift | c.462delT:c.462delT | p.Ala155fs:p.A |
| | | | | Ia155fs |
| 2:162310934:C:A | rs148590996 | splice_acceptor | c.454-1G > T:c.454-1G > T | |
| 2:162317853:AC:A | | splice_donor | c.453 + 1delG:c.453 + 1delG | |
| 2:162317854:C:T | rs967571395 | splice_donor | c.453 + 1G > A:c.453 + | |
| | | | 1G > A | |
| 2:162317869:C:A | | stop_gained | c.439G > T:c.439G > T | p.Glu147*:p.GI |
| | | | | u147* |
| 2:162317881:GT:G | | frameshift | c.426delA:c.426delA | p.Glu142fs:p.G |
| | | | | lu142fs |
| 2:162317890:C:A | | stop_gained | c.418G > T:c.418G > T | p.Glu140*:p.GI |
| | | | | u140* |
| 2:162317901:TC:T | rsl261022198 | frameshift | c.406delG:c.406delG | p.Asp136fs:p. |
| | | | | Aspl36fs |
| 2:162317944:G:A | | stop_gained | c.364C > T:c.364C > T | p.Gln122*:p.GI |
| | | | | nl22* |
| 2:162317962:G:A | | stop_gained | c.346C > T:c.346C > T | p.Gln116*:p.GI |
| | | | | nll6* |
| 2:162317965:GAT:G | | frameshift | c.341_342delAT:c.341_342delAT | p.Tyr114fs:p.T |
| | | | | yrl14fs |
| 2:162317966:A:C | | stop_gained | c.342T > G:c.342T > G | p.Tyr114*:p.Ty |
| | | | | rl14* |
| 2:162317967:TA:T | | frameshift | c.340delT:c.340delT | p.Tyr114fs:p.T |
| | | | | yrl14fs |
| 2:162317971:C:A | | stop_gained | c.337G > T:c.337G > T | p.Glu113*:p.GI |
| | | | | u113* |
| 2:162317986:C:A | | stop_gained | c.322G > T:c.322G > T | p.Glu108*:p.GI |
| | | | | u108* |
| 2:162317994:GGA:G | rs756183031 | frameshift | c.312_313delTC:c.312_313delTC | p.Pro105fs:p.P |
| | | | | ro105fs |
| 2:162318017:A:AG | | frameshift | c.290dupC:c.290dupC | p.Glu98fs:p.GI |
| | | | | u98fs |
| 2:162318026:G:T | | stop_gained | c.282C > A:c.282C > A | p.Tyr94*:p.Tyr |
| | | | | 94* |
| 2:162318055:T:A | | stop_gained | c.253A > T:c.253A > T | p.Arg85*:p.Ar |
| | | | | 885* |
| 2:162318065:C:CT | | frameshift | c.242dupA:c.242dupA | p.Ala82fs:p.AI |
| | | | | a82fs |
| 2:162318069:AC:A | | frameshift | c.238delG:c.238delG | p.Val80fs:p.Val |
| | | | | 80fs |
| 2:162318073:A:AT | rsl173976772 | frameshift | c.234dupA:c.234dupA | p.Phe79fs:p.P |
| | | | | he79fs |
| 2:162318084:CIT | | stop_gained | c.224G > A:c.224G > A | p.Trp75*:p.Trp |
| | | | | 75* |
| 2:162318092:GT:G | rsl327398341 | frameshift | c.215delA:c.215delA | p.His72fs:p.His |
| | | | | 72fs |
| 2:162318145:TC:T | rsl394699072 | frameshift | c.162delG:c.162delG | p.Asn55fs:p.As |
| | | | | n55fs |
| 2:162318169:G:A | | stop_gained | c.139C > T:c.139C > T | p.Gln47*:p.GI |
| | | | | n47* |
| 2:162318188:TGCAG:T | | frameshift | c.H6_119delCTGC: | p.Pro39fs:p.Pr |
| | | | c.H6_119delCTGC | o39fs |
| 2:162318254:GAA:G | | frameshift | c.52_53delTT:c.52_ | p.Phe18fs:p.P |
| | | | 53delTT | hel8fs |
| 2:162318254:GA:G | | frameshift | c.53delT:c.53delT | p.Phe18fs:p.P |
| | | | | hel8fs |
| 2:162318258:C:CA | | frameshift | c.49dupT:c.49dupT | p.Cys17fs:p.Cy |
| | | | | sl7fs |
| 2:162318283:C:A | | stop_gained | c.25G > T:c.25G > T | p.Glu9*:p.Glu |
| | | | | 9* |
| 2:162318284:GTC:G | | frameshift | c.22_23delGA:c.22_23delGA | p.Asp8fs:p.Asp |
| | | | | 8fs |
| 2:162318297:CCATTCGA:C | | frameshift | c.4_10delTCGAATG:c.4_ | p.Ser2fs:p.Ser |
| | | | 10delTCGAATG | 2fs |

In some embodiments, the subject's aggregate burden of having any one or more IFIH1 missense variant nucleic acid molecules encoding an IFIH1 predicted loss-of-function polypeptide represents a weighted sum of a plurality of any of the IFIH1 missense variant nucleic acid molecules encoding an IFIH1 predicted loss-of-function polypeptide. In some embodiments, the aggregate burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the IFIH1 gene where the genetic burden is the number of alleles multiplied by the association estimate with psoriasis or related outcome for each allele (e.g., a weighted polygenic burden score). This can include any genetic variants, regardless of their genomic annotation, in proximity to the IFIH1 gene (up to 10 Mb around the gene) that show a non-zero association with psoriasis-related traits in a genetic association analysis. In some embodiments, when the subject has an aggregate burden above a desired threshold score, the subject has a decreased risk of developing psoriasis. In some embodiments, when the subject has an aggregate burden below a desired threshold score, the subject has an increased risk of developing psoriasis.

In some embodiments, the aggregate burden may be divided into quintiles, e.g., top quintile, intermediate quintile, and bottom quintile, wherein the top quintile of aggregate burden corresponds to the lowest risk group and the bottom quintile of aggregate burden corresponds to the highest risk group. In some embodiments, a subject having a greater aggregate burden comprises the highest weighted aggregate burdens, including, but not limited to the top 10%, top 20%, top 30%, top 40%, or top 50% of aggregate burdens from a subject population. In some embodiments, the genetic variants comprise the genetic variants having association with psoriasis in the top 10%, top 20%, top 30%, top 40%, or top 50% of p-value range for the association. In some embodiments, each of the identified genetic variants comprise the genetic variants having association with psoriasis with p-value of no more than about 10⁻², about 10⁻³, about 10⁻⁴, about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³, about 10⁻¹⁴, about or 10⁻¹⁵. In some embodiments, the identified genetic variants comprise the genetic variants having association with psoriasis with p-value of less than 5×10⁻⁸. In some embodiments, the identified genetic variants comprise genetic variants having association with psoriasis in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects comprise subjects having aggregate burdens in the bottom decile, quintile, or tertile in a reference population. The threshold of the aggregate burden is determined on the basis of the nature of the intended practical application and the risk difference that would be considered meaningful for that practical application.

In some embodiments, when a subject is identified as having an increased risk of developing psoriasis, the subject is further treated with a therapeutic agent that treats or inhibits psoriasis, and/or an IFIH1 inhibitor, and/or a TRIM65 inhibitor, and/or type 1 interferon pathway inhibitor as described herein. For example, when the subject is IFIH1 reference, and therefore has an increased risk of developing psoriasis, the subject is administered an IFIH1 inhibitor. In some embodiments, such a subject is also administered a therapeutic agent that treats or inhibits psoriasis and/or a type 1 interferon pathway inhibitor and/or an IFIH1 inhibitor. In some embodiments, when the subject is heterozygous for an IFIH1 missense variant nucleic acid molecule encoding an IFIH1 predicted loss-of-function polypeptide, the subject is administered the therapeutic agent that treats or inhibits psoriasis in a dosage amount that is the same as or less than a standard dosage amount, and is also administered an IFIH1 inhibitor, and/or type 1 interferon pathway inhibitor, and/or an IFIH1 inhibitor. In some embodiments, the subject is IFIH1 reference. In some embodiments, the subject is heterozygous for an IFIH1 missense variant nucleic acid molecule encoding an IFIH1 predicted loss-of-function polypeptide. Furthermore, when the subject has a lower aggregate burden for having an IFIH1 missense variant nucleic acid molecule encoding an IFIH1 predicted loss-of-function polypeptide, and therefore has an increased risk of developing psoriasis, the subject is administered a therapeutic agent that treats or inhibits psoriasis. In some embodiments, when the subject has a lower aggregate burden for having an IFIH1 missense variant nucleic acid molecule encoding an IFIH1 predicted loss-of-function polypeptide, the subject is administered the therapeutic agent that treats or inhibits psoriasis in a dosage amount that is the same as or greater than the standard dosage amount administered to a subject who has a greater aggregate burden for having an IFIH1 missense variant nucleic acid molecule encoding an IFIH1 predicted loss-of-function polypeptide.

The present disclosure also provides methods of detecting the presence or absence of an IFIH1 or TRIM65 missense genomic variant nucleic acid molecule encoding an IFIH1 predicted loss-of-function polypeptide in a biological sample from a subject, and/or an IFIH1 or TRIM65 missense variant mRNA molecule encoding an IFIH1 or TRIM65 predicted loss-of-function polypeptide in a biological sample from a subject, and/or an IFIH1 or TRIM65 missense variant cDNA molecule encoding an IFIH1 or TRIM65 predicted loss-of-function polypeptide produced from an mRNA molecule in a biological sample from a subject. It is understood that gene sequences within a population and mRNA molecules encoded by such genes can vary due to polymorphisms such as single-nucleotide polymorphisms. The sequences provided herein for the IFIH1 or TRIM65 missense variant genomic nucleic acid molecule, IFIH1 or TRIM65 missense variant mRNA molecule, and IFIH1 or TRIM65 missense variant cDNA molecule are only exemplary sequences. Other sequences for the IFIH1 or TRIM65 missense variant genomic nucleic acid molecule, missense variant mRNA molecule, and missense variant cDNA molecule are also possible.

The biological sample can be derived from any cell, tissue, or biological fluid from the subject. The sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The sample used in the methods disclosed herein will vary based on the assay format, nature of the detection method, and the tissues, cells, or extracts that are used as the sample. A biological sample can be processed differently depending on the assay being employed. For example, when detecting any IFIH1 missense variant nucleic acid molecule, preliminary processing designed to isolate or enrich the sample for the genomic DNA can be employed. A variety of techniques may be used for this purpose. When detecting the level of any IFIH1 or TRIM65 missense variant mRNA, different techniques can be used enrich the biological sample with mRNA. Various methods to detect the presence or level of an mRNA or the presence of a particular missense variant genomic DNA locus can be used.

In some embodiments, detecting an IFIH1 or TRIM65 missense variant nucleic acid molecule encoding an IFIH1 or TRIM65 predicted loss-of-function polypeptide in a subject comprises assaying or genotyping a biological sample obtained from the subject to determine whether an IFIH1 or TRIM65 genomic nucleic acid molecule in the biological sample, and/or an IFIH1 or TRIM65 mRNA molecule in the biological sample, and/or an IFIH1 or TRIM65 cDNA molecule produced from an mRNA molecule in the biological sample, comprises one or more variations that cause a loss-of-function (partial or complete) or are predicted to cause a loss-of-function (partial or complete).

In some embodiments, the methods of detecting the presence or absence of an IFIH1 or TRIM65 missense variant nucleic acid molecule encoding an IFIH1 or TRIM65 predicted loss-of-function polypeptide (such as, for example, a genomic nucleic acid molecule, an mRNA molecule, and/or a cDNA molecule produced from an mRNA molecule) in a subject, comprise performing an assay on a biological sample obtained from the subject. The assay determines whether a nucleic acid molecule in the biological sample comprises a particular nucleotide sequence. In some embodiments, the IFIH1 nucleotide sequence comprises a cytosine at a position corresponding to position 38,690 according to SEQ ID NO:2 (for genomic nucleic acid molecules).

In some embodiments, the biological sample comprises a cell or cell lysate. Such methods can further comprise, for example, obtaining a biological sample from the subject comprising an IFIH1 or TRIM65 genomic nucleic acid molecule or mRNA molecule, and if mRNA, optionally reverse transcribing the mRNA into cDNA. Such assays can comprise, for example determining the identity of these positions of the particular IFIH1 or TRIM65 nucleic acid molecule. In some embodiments, the method is an in vitro method.

In some embodiments, the determining step, detecting step, or sequencing analysis comprises sequencing at least a portion of the nucleotide sequence of the IFIH1 or TRIM65 genomic nucleic acid molecule, the IFIH1 or TRIM65 mRNA molecule, or the IFIH1 or TRIM65 cDNA molecule in the biological sample, wherein the sequenced portion comprises one or more variations that cause a loss-of-function (partial or complete) or are predicted to cause a loss-of-function (partial or complete).

In some embodiments, the determining step, detecting step, or sequence analysis comprises sequencing at least a portion of the nucleotide sequence of the IFIH1 genomic nucleic acid molecule in the biological sample, wherein the sequenced portion comprises a position corresponding to position 38,690 according to SEQ ID NO:2, or the complement thereof. When the sequenced portion of the IFIH1 nucleic acid molecule in the biological sample comprises a cytosine at a position corresponding to position 38,690 according to SEQ ID NO:2, then the IFIH1 nucleic acid molecule in the biological sample is an IFIH1 missense variant nucleic acid molecule encoding an IFIH1 predicted loss-of-function polypeptide.

In some embodiments, the determining step, detecting step, or sequence analysis comprises: a) contacting the biological sample with a primer hybridizing to a portion of the nucleotide sequence of the IFIH1 genomic nucleic acid molecule that is proximate to a position corresponding to position 38,690 according to SEQ ID NO:2; b) extending the primer at least through the position of the nucleotide sequence of the IFIH1 genomic nucleic acid molecule corresponding to position 38,690 according to SEQ ID NO:2; and c) determining whether the extension product of the primer comprises a cytosine at a position corresponding to position 38,690 according to SEQ ID NO:2.

In some embodiments, the assay comprises sequencing the entire nucleic acid molecule. In some embodiments, only an IFIH1 or TRIM65 genomic nucleic acid molecule is analyzed. In some embodiments, only an IFIH1 or TRIM65 mRNA is analyzed. In some embodiments, only an IFIH1 cDNA obtained from IFIH1 or TRIM65 mRNA is analyzed.

In some embodiments, the determining step, detecting step, or sequence analysis comprises: a) amplifying at least a portion of the nucleic acid molecule that encodes the human IFIH1 polypeptide, wherein the amplified portion comprises a cytosine at a position corresponding to position 38,690 according to SEQ ID NO:2, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a cytosine at a position corresponding to position 38,690 according to SEQ ID NO:2, or the complement thereof; and d) detecting the detectable label.

In some embodiments, the nucleic acid molecule is mRNA and the determining step further comprises reverse-transcribing the mRNA into a cDNA prior to the amplifying step.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising a cytosine at a position corresponding to position 38,690 according to SEQ ID NO:2, or the complement thereof; and detecting the detectable label.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the human subject.

In some embodiments, the assay comprises contacting the biological sample with a primer or probe, such as an alteration-specific primer or alteration-specific probe, that specifically hybridizes to an IFIH1 or TRIM65 variant genomic sequence, variant mRNA sequence, or variant cDNA sequence and not the corresponding IFIH1 or TRIM65 reference sequence under stringent conditions, and determining whether hybridization has occurred.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising an IFIH1 or TRIM65 missense variant genomic nucleic acid molecule, missense variant mRNA molecule, or missense variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

In some embodiments, to determine whether an IFIH1 nucleic acid molecule (genomic nucleic acid molecule, mRNA molecule, or cDNA molecule), or complement thereof, within a biological sample comprises a nucleotide sequence comprising a cytosine at a position corresponding to position 38,690 according to SEQ ID NO:2 (genomic nucleic acid molecule), the biological sample can be subjected to an amplification method using a primer pair that includes a first primer derived from the 5′ flanking sequence adjacent to a cytosine at a position corresponding to position 38,690 according to SEQ ID NO:2, and a second primer derived from the 3′ flanking sequence adjacent to a cytosine at a position corresponding to position 38,690 according to SEQ ID NO:2 to produce an amplicon that is indicative of the presence of the SNP at positions encoding a cytosine at a position corresponding to position 38,690 according to SEQ ID NO:2. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a cytosine at a position corresponding to position 38,690 according to SEQ ID NO:2 and at least 1, 2, 3, 4, 5, 6, 7, 8, 9, 10, or more nucleotides on each side of positions comprising a cytosine at a position corresponding to position 38,690 according to SEQ ID NO:2.

Similar amplicons can be generated from the mRNA and/or cDNA sequences. PCR primer pairs can be derived from a known sequence, for example, by using computer programs intended for that purpose, such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned and primers manually identified using known guidelines.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The present disclosure also provides methods of detecting the presence of an IFIH1 predicted loss-of-function polypeptide comprising performing an assay on a sample obtained from a subject to determine whether an IFIH1 polypeptide in the subject contains one or more variations that causes the polypeptide to have a loss-of-function (partial or complete) or predicted loss-of-function (partial or complete). The IFIH1 predicted loss-of-function polypeptide can be any of the IFIH1 truncated variant polypeptides described herein. In some embodiments, when the subject does not have an IFIH1 predicted loss-of-function polypeptide, then the subject has an increased risk for developing psoriasis. In some embodiments, when the subject has an IFIH1 predicted loss-of-function polypeptide, then the subject has a decreased risk for developing psoriasis.

The present disclosure also provides methods of detecting the presence of a TRIM65 predicted loss-of-function polypeptide comprising performing an assay on a sample obtained from a subject to determine whether a TRIM65 polypeptide in the subject contains one or more variations that causes the polypeptide to have a loss-of-function (partial or complete) or predicted loss-of-function (partial or complete). The TRIM65 predicted loss-of-function polypeptide can be any of the TRIM65 variant polypeptides described herein. In some embodiments, when the subject does not have a TRIM65 predicted loss-of-function polypeptide, then the subject has an increased risk for developing psoriasis. In some embodiments, when the subject has a TRIM65 predicted loss-of-function polypeptide, then the subject has a decreased risk for developing psoriasis.

The present disclosure also provides isolated nucleic acid molecules that hybridize to IFIH1 or TRIM65 missense variant genomic nucleic acid molecules, IFIH1 or TRIM65 missense variant mRNA molecules, and/or IFIH1 or TRIM65 missense variant cDNA molecules (such as any of the genomic variant nucleic acid molecules, mRNA variant molecules, and cDNA variant molecules disclosed herein). In some embodiments, the isolated nucleic acid molecules hybridize to a portion of the IFIH1 nucleic acid molecule that includes a position corresponding to position 38,690 according to SEQ ID NO:2.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to IFIH1 or TRIM65 missense variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein, and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to IFIH1 or TRIM65 missense variant genomic nucleic acid molecules, IFIH1 or TRIM65 missense variant mRNA molecules, and/or IFIH1 or TRIM65 missense variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the isolated alteration-specific probes or alteration-specific primers comprise at least about 15 nucleotides, wherein the alteration-specific probe or alteration-specific primer comprises a nucleotide sequence which is complementary to a portion of an IFIH1 missense variant nucleic acid molecule encoding an IFIH1 predicted loss-of-function polypeptide, wherein the portion comprises a position corresponding to position 38,690 according to SEQ ID NO:2, or the complement thereof.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the IFIH1 or TRIM65 missense variant genomic nucleic acid molecules, IFIH1 or TRIM65 missense variant mRNA molecules, and/or IFIH1 or TRIM65 missense variant cDNA molecules disclosed herein. The primers described herein can be used to amplify IFIH1 or TRIM65 missense variant genomic nucleic acid molecules, IFIH1 or TRIM65 missense variant mRNA molecules, or IFIH1 or TRIM65 missense variant cDNA molecules, or a fragment thereof.

The present disclosure also provides pairs of primers comprising any of the primers described above. For example, if one of the primers' 3′-ends hybridizes to a guanine at a position corresponding to position 38,690 according to SEQ ID NO:1 (rather than cytosine) in a particular IFIH1 nucleic acid molecule, then the presence of the amplified fragment would indicate the presence of an IFIH1 reference genomic nucleic acid molecule. Conversely, if one of the primers' 3′-ends hybridizes to a cytosine at a position corresponding to position 38,690 according to SEQ ID NO:2 (rather than guanine) in a particular IFIH1 nucleic acid molecule, then the presence of the amplified fragment would indicate the presence of the IFIH1 missense variant genomic nucleic acid molecule. In some embodiments, the nucleotide of the primer complementary to the cytosine at a position corresponding to position 38,690 according to SEQ ID NO:2 can be at the 3′ end of the primer.

In the context of the disclosure “specifically hybridizes” means that the probe or primer (such as, for example, the alteration-specific probe or alteration-specific primer) does not hybridize to a nucleic acid sequence encoding an IFIH1 or TRIM65 reference genomic nucleic acid molecule, an IFIH1 or TRIM65 reference mRNA molecule, and/or an IFIH1 or TRIM65 reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The nucleotide sequence of an IFIH1 reference genomic nucleic acid molecule is set forth in SEQ ID NO:1. This sequence corresponds to chromosome 2 positions 162,267,074-162,318,684 according to GRCh38/hg38 human genome assembly (Gencode gene ENSG00000115267.8). Referring to SEQ ID NO:1, position 38,690 is a guanine.

A variant genomic nucleic acid molecule of IFIH1 exists, wherein the guanine at position 38,690 is replaced with cytosine. The nucleotide sequence of this IFIH1 variant genomic nucleic acid molecule is set forth in SEQ ID NO:2.

The nucleotide sequence of an IFIH1 reference mRNA molecule is set forth in SEQ ID NO: 3. The nucleotide sequence of another IFIH1 reference mRNA molecule is set forth in SEQ ID NO:4. The nucleotide sequence of another IFIH1 reference mRNA molecule is set forth in SEQ ID NO:5. The nucleotide sequence of another IFIH1 reference mRNA molecule is set forth in SEQ ID NO:6. The nucleotide sequence of another IFIH1 reference mRNA molecule is set forth in SEQ ID NO:7. The nucleotide sequence of another IFIH1 reference mRNA molecule is set forth in SEQ ID NO:8. The nucleotide sequence of another IFIH1 reference mRNA molecule is set forth in SEQ ID NO:9 The nucleotide sequence of another IFIH1 reference mRNA molecule is set forth in is set forth in SEQ ID NO:10.

The nucleotide sequence of an IFIH1 reference cDNA molecule is set forth in SEQ ID NO: 11. The nucleotide sequence of another IFIH1 reference cDNA molecule is set forth in SEQ ID NO:12. The nucleotide sequence of another IFIH1 reference cDNA molecule is set forth in SEQ ID NO:13. The nucleotide sequence of another IFIH1 reference cDNA molecule is set forth in SEQ ID NO:14. The nucleotide sequence of another IFIH1 reference cDNA molecule is set forth in SEQ ID NO:15. The nucleotide sequence of another IFIH1 reference cDNA molecule is set forth in SEQ ID NO:16. The nucleotide sequence of another IFIH1 reference cDNA molecule is set forth in SEQ ID NO:17. The nucleotide sequence of another IFIH1 reference cDNA molecule is set forth in SEQ ID NO:18.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1, SEQ ID NO:3, or SEQ ID NO:12). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

For example, a nucleic acid molecule comprising a nucleotide sequence encoding an IFIH1 polypeptide, wherein the nucleotide sequence comprises a cytosine at a position corresponding to position 38,690 according to SEQ ID NO:2 means that if the nucleotide sequence of the IFIH1 genomic nucleic acid molecule is aligned to the sequence of SEQ ID NO: 2, the IFIH1 sequence has a guanine residue at the position that corresponds to position 38,690 of SEQ ID NO:2. In other words, these phrases refer to a nucleic acid molecule encoding an IFIH1 polypeptide, wherein the genomic nucleic acid molecule has a nucleotide sequence that comprises a guanine residue that is homologous to the guanine residue at position 38,690 of SEQ ID NO:2.

As described herein, a position within an IFIH1 genomic nucleic acid molecule that corresponds to position 38,690 according to SEQ ID NO:2, for example, can be identified by performing a sequence alignment between the nucleotide sequence of a particular IFIH1 nucleic acid molecule and the nucleotide sequence of SEQ ID NO:2. A variety of computational algorithms exist that can be used for performing a sequence alignment to identify a nucleotide position that corresponds to, for example, position 38,690 in SEQ ID NO: 2. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

The amino acid sequence of an IFIH1 reference polypeptide is set forth in SEQ ID NO: 19, and is 1,025 amino acids in length. The amino acid sequence of another IFIH1 reference polypeptide is set forth in SEQ ID NO:20, and is 986 amino acids in length. The amino acid sequence of another IFIH1 reference polypeptide is set forth in SEQ ID NO:21, and is 468 amino acids in length. The amino acid sequence of another IFIH1 reference polypeptide is set forth in SEQ ID NO:22, and is 772 amino acids in length. The amino acid sequence of another IFIH1 reference polypeptide is set forth in SEQ ID NO:23, and is 221 amino acids in length.

The present disclosure also provides therapeutic agents that treat or inhibit psoriasis for use in the treatment of psoriasis in a subject having a genomic nucleic acid molecule having a nucleotide sequence encoding an IFIH1 predicted loss-of-function polypeptide. In some embodiments, the nucleic acid molecule comprises a nucleotide sequence comprising a cytosine at a position corresponding to position 38,690 according to SEQ ID NO:2, or the complement thereof. The therapeutic agent that treats or inhibits psoriasis can be any of the therapeutic agents described herein.

The present disclosure also provides therapeutic agents that treat or inhibit psoriasis for use in the preparation of a medicament for treating psoriasis in a subject having a genomic nucleic acid molecule having a nucleotide sequence encoding an IFIH1 predicted loss-of-function polypeptide. In some embodiments, the nucleic acid molecule comprises a nucleotide sequence comprising a cytosine at a position corresponding to position 38,690 according to SEQ ID NO:2, or the complement thereof. The therapeutic agent that treats or inhibits psoriasis can be any of the therapeutic agents described herein.

The present disclosure also provides IFIH1 inhibitors for use in the treatment of psoriasis in a subject that is IFIH1 reference or heterozygous for an IFIH1 missense variant nucleic acid molecule encoding an IFIH1 predicted loss-of-function polypeptide. In some embodiments, the nucleic acid molecule comprises a nucleotide sequence comprising a cytosine at a position corresponding to position 38,690 according to SEQ ID NO:2, or the complement thereof. The IFIH1 inhibitor can be any of the IFIH1 inhibitors described herein.

The present disclosure also provides IFIH1 inhibitors for use in the preparation of a medicament for treating psoriasis in a subject that is IFIH1 reference or heterozygous for an IFIH1 missense variant nucleic acid molecule encoding an IFIH1 predicted loss-of-function polypeptide. In some embodiments, the nucleic acid molecule comprises a nucleotide sequence comprising a cytosine at a position corresponding to position 38,690 according to SEQ ID NO:2, or the complement thereof. The IFIH1 inhibitor can be any of the IFIH1 inhibitors described herein.

The present disclosure also provides therapeutic agents that treat or inhibit psoriasis for use in the treatment of psoriasis in a subject that is TRIM65 reference or heterozygous for a TRIM65 missense variant nucleic acid molecule encoding a TRIM65 predicted loss-of-function polypeptide. In some embodiments, the TRIM65 predicted loss-of-function polypeptide is TRIM65 Gly382Arg. The therapeutic agent that treats or inhibits psoriasis can be any of the therapeutic agents described herein.

The present disclosure also provides therapeutic agents that treat or inhibit psoriasis for use in the preparation of a medicament for treating psoriasis in a subject that is TRIM65 reference or heterozygous for a TRIM65 missense variant nucleic acid molecule encoding a TRIM65 predicted loss-of-function polypeptide. In some embodiments, the TRIM65 predicted loss-of-function polypeptide is TRIM65 Gly382Arg. The therapeutic agent that treats or inhibits psoriasis can be any of the therapeutic agents described herein.

The present disclosure also provides TRIM65 inhibitors for use in the treatment of psoriasis in a subject that is TRIM65 reference or heterozygous for a TRIM65 missense variant nucleic acid molecule encoding a TRIM65 predicted loss-of-function polypeptide. In some embodiments, the TRIM65 predicted loss-of-function polypeptide is TRIM65 Gly382Arg. The TRIM65 inhibitor can be any of the TRIM65 inhibitors described herein.

The present disclosure also provides TRIM65 inhibitors for use in the preparation of a medicament for treating psoriasis in a subject that is TRIM65 reference or heterozygous for a TRIM65 missense variant nucleic acid molecule encoding a TRIM65 predicted loss-of-function polypeptide. In some embodiments, the TRIM65 predicted loss-of-function polypeptide is TRIM65 Gly382Arg. The TRIM65 inhibitor can be any of the TRIM65 inhibitors described herein.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: A Haplotype Carrying a Common Missense Variant in IFIH1 is Associated with Psoriasis Phenotypes in a Protective Direction

A meta analysis of several genetic cohorts (Table 3) was carried out to identify novel genetic associations.

TABLE 3
Number of Psoriasis cases/controls
sequenced and analyzed, to date (EUR)

| | Number of | Number of |
|---|---|---|
| Cohort | Cases | Controls |
| UK Biobank | 10,715 | 458,650 |
| GHS 145K Mega | 4,044 | 122,132 |
| Kiel | 1,390 | 1,206 |
| Sinai BioMe | 361 | 22,417 |
| Penn Med BioBank | 212 | 15,276 |
| Michigan (J. T. Elder) | 4,088 | 3,478 |
| Total | 20,810 | 623,159 |

Significant common variant associations were found in known loci, including IFIH1; in total 5 common, independent/novel signals in known loci (data not shown). In addition, independent and potentially novel common variant signals in/near known GWAS regions have been identified (Table 4; gene=IFIH1; phenotype=psoriasis meta). Row 1: variant=2:162267541:C:T; rsID=rs1990760; and HGVS=missense Ala946Thr. Row 2: variant=2:162268127:T:C; rsID=rs35667974; and HGVS=missense Ile923Val. Row 3: variant=2:162279995:C:G; rsID=rs35337543; and HGVS=splice_donor c.1641+1G>C). Row 4: variant=2:162352383:T:G; rsID=rs17783344; and HGVS=missense Ser80Ala.

TABLE 4
Independent and potentially novel common variant signals in/near
psoriasis GWAS loci

| Effect | | | | | | |
|---|---|---|---|---|---|---|
| (95% | P- | Cases | Controls | | Heterozygote | Homozygote |
| CI) | value | RR\|RA\|AA | RR\|RA\|AA | AAF | OR (95% CI) | OR (95% CI) |
| 0.850 | 5.18e−15 | 7798\|9064\|2974 | 202572\|271656\|106593 | 0.412 | 0.867 | 0.725 |
| (0.832, | | | | | (0.840, 0.894) | (0.694, 0.757) |
| 0.867) | | | | | | |
| 0.718 | 3.02e−11 | 19141\|536\|6 | 558537\|21952\|227 | 0.019 | 0.712 | 0.771 |
| (0.659, | | | | | (0.653, 0.777) | (0.343, 1.735) |
| 0.782) | | | | | | |
| 0.676 | 2.64e−8 | 19337\|333\|2 | 565784\|14433\|112 | 0.012 | 0.675 | 0.522 |
| (0.606, | | | | | (0.605, 0.753) | (0.129, 2.115) |
| 0.753) | | | | | | |
| 0.938 | 1.73e−7 | 15201\|4265\|312 | 438987\|129943\|10769 | 0.130 | 0.948 | 0.837 |
| (0.910, | | | | | (0.916, 0.981) | (0.747, 0.937) |
| 0.967) | | | | | | |

Specifically, the analysis revealed a protective IFIH1 splice variant association to psoriasis that is both novel and independent of IFIH missense variants known to associate with reduced odds of psoriasis.

The psoriasis meta-analysis also shown significant associations of IFIH1 gene burden results reduced odds of psoriasis: additional IFIH1 pLoFs and rare missense variants contribute to protection (Table 5; gene=IFIH1).

TABLE 5
Additional rare pLoFs/deleterious missense variants in IFIH1 associations with reduced odds of psoriasis

| | | | Cases | Controls | |
|---|---|---|---|---|---|
| Mask | Effect (95% Cl) | P-value | RR\|RA\|AA | RR\|RA\|AA | AAF |
| M1.1 | 0.742 (0.683, 0.808) | 4.05E−12 | 19196\|619\|3 | 558193\|22548\|7 | 0.019 |
| M1.01 | 0.673 (0.523, 0.866) | 2.05E−3 | 19519\|57\|0 | 566202\|2501\|0 | 0.002 |
| M1.001 | 0.705 (0.501, 0.994) | 4.60E−2 | 18159\|26\|0 | 566202\|1295\|0 | 0.001 |
| M3.1 | 0.793 (0.738, 0.852) | 2.92E−19 | 18928\|887\|3 | 548960\|3176\|100 | 0.027 |
| M3.01 | 0.851 (0.746, 0.971) | 1.66E−2 | 19435\|276\|0 | 559785\|9034\|7 | 0.008 |
| M3.001 | 0.743 (0.606,0.911) | 4.36E−3 | 19494\|82\|0 | 565096\|3610\|0 | 0.003 |

Functional Prediction=pLoFs, <1% AAF (data row 1); pLoFs, <0.1% AAF (data row 2); pLoFs, <0.01% AAF (data row 3); pLoFs and deleterious missense, <1% AAF (data row 4); pLoFs and deleterious missense, <0.1% AAF (data row 5); and pLoFs and deleterious missense, <0.01% AAF (data row 6).

Moreover, significant gene-burden associations for three genes in the Type 1 interferon pathway: IFIH1, ADAR, TRIM65 were also shown. (Table 6; gene=IFIH1 (data rows 1 and 2), ADAR (data rows 3 and 4), and TRIM65 (data rows 5 and 6)).

TABLE 6
Significant M1/M3 gene-burden associations for genes in Type 1
interferon pathway

| | | Cases | Controls | |
|---|---|---|---|---|
| Effect (95% Cl) | P-value | RR\|RA\|AA | RR\|RA\|AA | AAF |
| 0.742 | 4.05E−12 | 19196\|619\|3 | 558193122548\|7 | 0.019 |
| (0.683, 0.808) | | | | |
| 0.793 | 2.92E−10 | 18928\|887\|3 | 548960\|3176\|100 | 0.027 |
| (0.738, 0.852) | | | | |
| 3.741 | 2.02E−4 | 17863\|16\|0 | 539839\|162\|0 | 0.0002 |
| (1.866, 7.501) | | | | |
| 2.292 | 1.36E−7 | 18218\|74\|0 | 578560\|933\|0 | 0.0008 |
| (1.683, 3.120) | | | | |
| 0.697 | 1.28E−2 | 18253\|39\|0 | 577669\|1824\|0 | 0.002 |
| (0.524, 0.926) | | | | |
| 0.628 | 4.78E−5 | 18350\|77\|0 | 575397\|4214\|0 | 0.004 |
| (0.502, 0.786) | | | | |

Functional Prediction=pLoFs, <1% AAF (M1)(data row 1); pLoFs and deleterious missense, <1% AAF (M3)(data row 2); pLoFs, <1% AAF (M1)(data row 3); pLoFs and deleterious missense, <1% AAF (M3)(data row 4); pLoFs, <1% AAF (M1) (data row 5); and pLoFs and deleterious missense, <1% AAF (M3)(data row 6).

Furthermore, additional rare pLoFs/deleterious missense variants in TRIM65 associate with reduced odds of psoriasis (Table 7).

TABLE 7
Additional rare pLoFs/deleterious missense variants in TRIM65 associate
with reduced odds of psoriasis

| | | Cases | Controls | |
|---|---|---|---|---|
| Effect (95% Cl) | P-value | RR\|RA\|AA | RR\|RA\|AA | AAF |
| 0.697 | 1.28E−2 | 18253\|39\|0 | 577669\|1824\|0 | 0.002 |
| (0.525, 0.926) | | | | |
| 0.628 | 4.78E−5 | 18350\|77\|0 | 575397\|4214\|2 | 0.004 |
| (0.502, 0.786) | | | | |
| 0.413 | 1.47E−4 | 18107\|11\|0 | 568492\|1074\|1 | 0.0009 |
| (0.262, 0.652) | | | | |

Functional Prediction=pLoFs, <1% AAF (M1)(data row 1); pLoFs and deleterious missense, <1% AAF (M3)(data row 2); and Missense p.Gly382Arg; 17:75891189:C:T (data row 3).

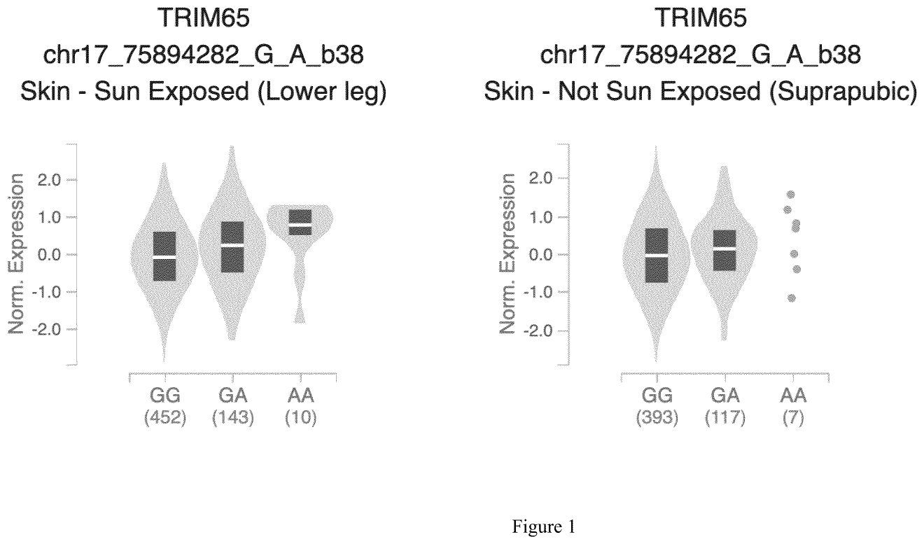

Finally, the data suggests that TRIM65 inhibition may be protective in psoriasis (Table 8; Variant=17:75894282:G:A (data row 1) and 17:75891189:C:T (data row 2); rsID=rs55823223 (data row 1) and rs202175254 (data row 2); and HGVS=Intronic c.415-1432C>T (data row 1) and Missense p.Gly382Arg (data row 2); and FIG. 1).

TABLE 8
Common TRIM65 psoriasis variant is an eQTL
for increased TRIM65 expression

| | | | Heterozygote | Homozygote |
|---|---|---|---|---|
| Effect | | | OR | OR (95% |
| (95% Cl) | P-value | AAF | (95% Cl) | Cl) |
| 1.11 | 4.09E−6 | 0.14 | 1.12 | 1.22 |
| (1.07, 1.16) | | | (1.07, 1.17) | (1.07, 1.38) |
| 0.413 | 1.47E−4 | 0.0009 | NA | NA |
| (0.262, 0.652) | | | | |

Cases RR|RA|AA=7092|2603|247 (data row 1), and 18107|11|0 (data row 2).
Controls RR|RA|AA=313749|102655|8980 (data row 1), and 568492|1074|1 (data row 2).

The meta analysis also revealed a significant associations for rare pLoFs/deleterious heterozygous missense variants in ADAR1 and psoriasis (Table 9).

TABLE 9
Significant associations for rare pLoFs/deleterious heterozygous
missense variants in ADARI and psoriasis

| | | Cases | Controls | |
|---|---|---|---|---|
| Effect (95% Cl) | P-value | RR\|RA\|AA | RR\|RA\|AA | AAF |
| 3.741 | 2.02E−4 | 17863\|16\|0 | 539839\|162\|0 | 0.0002 |
| (1.866, 7.501) | | | | |
| 2.292 | 1.36E−7 | 18218\|74\|0 | 578560\|933\|0 | 0.0008 |
| (1.683, 3.120) | | | | |

Functional Prediction=pLoFs, <1% AAF (M1)(data row 1); and pLoFs and deleterious missense, <1% AAF (M3)(data row 2).

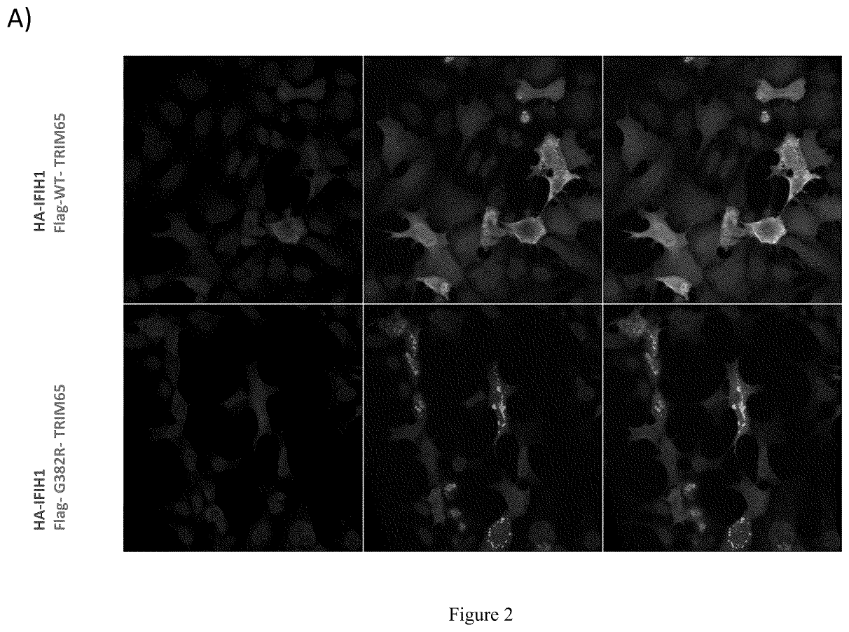

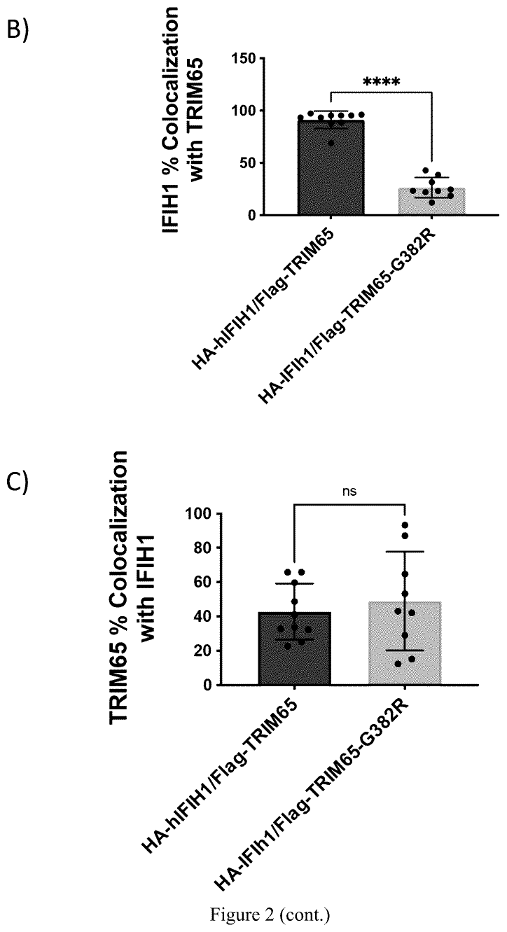

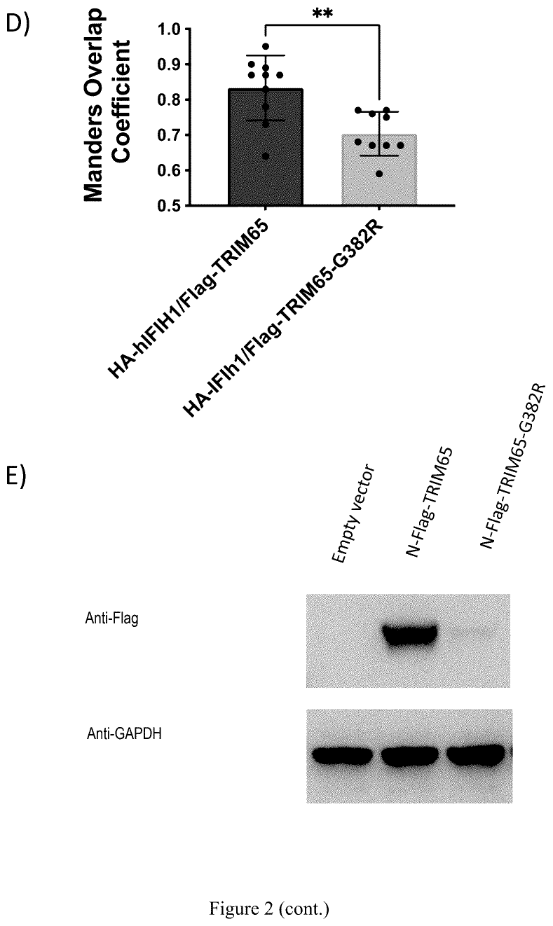

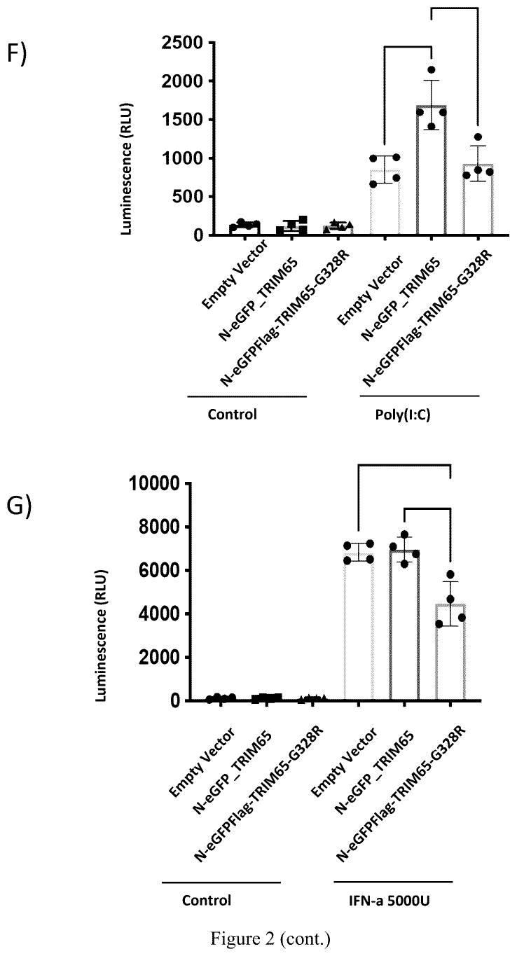

### Example 2: TRIM65-G382R Variant Alters the Cellular Localization and Expression Levels of TRIM65 (FIG. 2)

### Cell Culture, Plasmids and Cell Transfection:

HEK293-HZ cells were maintained in Dulbecco's modified Eagle's medium supplemented with 10% fetal bovine serum, and antibiotics (50 units/mL penicillin and 50 μg/ml streptomycin; Thermo Fisher Scientific). pcDNA 3.1 plasmids encoding for N-terminus Flag-tagged wild-type TRIM65 or TRIM65-G382R, and N-terminally HA-tagged wild-type IFIH1 were synthesized by GenScript (USA). Cells at approximately 60-70% confluence were transiently transfected using FuGENE 6 (Promega) according to the manufacturer's protocol, at a ratio of 1 μg DNA:5 ul FuGENE transfection reagent. After 48 hours, cells were washed with 1×DPBS (Thermo Fisher Scientific) and collected for downstream analysis.

### Immunofluorescence Assays:

For immunofluorescence assays, cells were seeded onto open 8-well μ-Slides (chamber slide) with a glass bottom (Ibidi, cat #80827). The following day, cells were transfected with pcDNA 3.1 plasmids encoding for N-terminally Flag-tagged wild-type TRIM65 or TRIM65-G382R constructs, with or without co-transfection of N-terminally HA-tagged WT IFIH1. At 48 hours post-transfection, cells were fixed in ice-cold 4% PFA for 10 minutes at RT and washed 3× with ice-cold 1×DPBS (all subsequent wash steps performed 3 times with ice-cold 1×DPBS for 5 minutes per wash). Cells were blocked using 10% normal donkey serum (NDS) with 0.1% Triton X-100 (Jackson Immunoresearch Laboratories, #017-000-121). Cells were incubated with 1:2000 anti-Flag antibody (Sigma) and anti-HA antibody (Cell Signaling) O/N, washed and then incubated for 1 hour with 1:1000 Alexa Fluor 594-conjugated anti-mouse secondary antibody and 1:1000 Alexa Fluor 647-conjugated anti-rabbit secondary antibody (Thermo Fisher Scientific). Wells were then washed, and slides were mounted with ProLong® Gold Antifade Reagent with DAPI (Cell Signaling, #8961). Slides were imaged using Zeiss confocal LSM880. Colocalization coefficients and the Mander's Overlap Coefficient were calculated using ZEN Blue. Thresholds for each channel were estimated from single-labeled control wells.

### Western Blotting:

48 hours after transfection in 10 cm² cell culture plates, HEK293HZ cells were pelleted and lysed in RIPA buffer supplemented with protease and kinase inhibitors. The following primary antibodies were used: anti-Flag M2 (mouse monoclonal, Sigma) and GAPDH 14C10 (Rabbit mAb, Cell Signaling Cat #2118). The appropriate LI-COR secondary IRDye antibodies (anti-rabbit (926-32211) and anti-mouse (926-32210)) were used to detect and quantify immunoblots using a LI-COR Odyssey Infrared Imaging System (LI-COR, Lincoln, NE).

### Quantification of ISRE Activity:

HEK293-ISRE-luc cells were maintained in Dulbecco's modified Eagle's medium supplemented with 10% fetal bovine serum (FBS), and antibiotics (50 units/mL penicillin and 50 μg/mL streptomycin; Thermo Fisher Scientific), 1×NEAA, and 1×L-Glutamine. pcDNA 3.1 plasmids encoding for N-terminus eGFP-tagged wild-type TRIM65 or TRIM65-G382R were synthesized by GenScript (USA). Cells at approximately 60-70% confluence were transiently transfected using FuGENE 6 (Promega) according to the manufacturer's protocol, at a ratio of 1 μg DNA:5 μl FuGENE transfection reagent. The following day, serum in the media was reduced to 0.5% FBS, and 24 hours after transfection, cells were stimulated with HMW 0.05 μg poly(i:c)(Invivogen) or 5000 U human IFN-α (R & D Systems) overnight. Luciferase activity was assessed using the Bright-Glo Luciferase Assay System (Promega) according to the manufacturer's protocol and read on a SpectraMax® i3x Multi-Mode Microplate Reader.

The obtained results suggest that the TRIM65-G382R variant alters the cellular localization and expression levels of TRIM65. TRIM65-G382R displays reduced colocalization with its binding partner IFIH1. This observation, along with reduced interferon-stimulated response element (ISRE) activity in response to stimulation by IFN-α or poly(i:c)(an analog for dsRNA which is known to activate IFIH1 in vitro) in cells transfected with a TRIM65-G382R construct, suggest that this variant likely leads to reduced interferon pathway activation, which may in turn be protective against psoriasis.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1. A method of treating a subject having psoriasis, the method comprising administering an Interferon Induced Helicase C Domain 1 (IFIH1) inhibitor to the subject.

2. The method according to claim 1, wherein the psoriasis comprises guttate psoriasis, plaque psoriasis, inverse psoriasis, pustular psoriasis, or erythrodermic psoriasis.

3-6. (canceled)

7. The method according to claim 1, the method further comprising administering a type 1 interferon pathway inhibitor and/or a Tripartite Motif Containing 65 (TRIM65) inhibitor to the subject.

8. (canceled)

9. The method according to claim 1, wherein the IFIH1 inhibitor comprises an inhibitory nucleic acid molecule, wherein the inhibitory nucleic acid molecule comprises an antisense nucleic acid molecule, a small interfering RNA (siRNA), or a short hairpin RNA (shRNA) that hybridizes to an IFIH1 mRNA, or the IFIH1 inhibitor comprises paramyxovirus V protein.

10-18. (canceled)

19. The method according to claim 1, wherein the subject is IFIH1 reference or is heterozygous for an IFIH1 missense variant nucleic acid molecule encoding an IFIH1 predicted loss-of-function polypeptide.

20. The method according to claim 19, wherein the IFIH1 missense variant nucleic acid molecule encoding an IFIH1 predicted loss-of-function polypeptide is a genomic nucleic acid molecule having a nucleotide sequence comprising a cytosine at a position corresponding to position 38,690 according to SEQ ID NO:2.

21-26. (canceled)

27. A method of treating a subject with a therapeutic agent that treats or inhibits psoriasis and/or an Interferon Induced Helicase C Domain 1 (IFIH1) inhibitor, wherein the subject has psoriasis, the method comprising the steps of:
determining whether the subject has an IFIH1 missense variant nucleic acid molecule encoding an IFIH1 predicted loss-of-function polypeptide by:
obtaining or having obtained a biological sample from the subject; and
performing or having performed a sequence analysis on the biological sample to determine if the subject has a genotype comprising the IFIH1 missense variant nucleic acid molecule; and
administering or continuing to administer the therapeutic agent that treats or inhibits psoriasis in a standard dosage amount to a subject that is IFIH1 reference, and/or administering an IFIH1 inhibitor to the subject;
administering or continuing to administer the therapeutic agent that treats or inhibits psoriasis in an amount that is the same as or less than a standard dosage amount to a subject that is heterozygous for the IFIH1 missense variant nucleic acid molecule, and/or administering an IFIH1 inhibitor to the subject; or
administering or continuing to administer the therapeutic agent that treats or inhibits psoriasis in an amount that is the same as or less than a standard dosage amount to a subject that is homozygous for the IFIH1 missense variant nucleic acid molecule;
wherein the presence of a genotype having the IFIH1 missense variant nucleic acid molecule encoding the IFIH1 predicted loss-of-function polypeptide indicates the subject has a decreased risk of developing psoriasis.

28. The method according to claim 27, wherein the subject is IFIH1 reference, and the subject is administered or continued to be administered the therapeutic agent that treats or inhibits psoriasis in a standard dosage amount, and/or is administered an IFIH1 inhibitor.

29. The method according to claim 27, wherein the subject is heterozygous for an IFIH1 missense variant nucleic acid molecule, and the subject is administered or continued to be administered the therapeutic agent that treats or inhibits psoriasis in an amount that is the same as or less than a standard dosage amount, and/or is administered an IFIH1 inhibitor.

30. The method according to claim 27, further comprising administering a type 1 interferon pathway inhibitor and/or a Tripartite Motif Containing 65 (TRIM65) inhibitor to the subject.

31. The method according to claim 27, wherein the IFIH1 missense variant nucleic acid molecule encoding an IFIH1 predicted loss-of-function polypeptide is a genomic nucleic acid molecule having a nucleotide sequence comprising a cytosine at a position corresponding to position 38,690 according to SEQ ID NO:2.

32-38. (canceled)

39. The method according to claim 27, wherein the IFIH1 inhibitor comprises an inhibitory nucleic acid molecule, wherein the inhibitory nucleic acid molecule comprises an antisense nucleic acid molecule, a small interfering RNA (siRNA), or a short hairpin RNA (shRNA) that hybridizes to an IFIH1 mRNA, or the IFIH1 inhibitor comprises paramyxovirus V protein.

40-46. (canceled)

47. A method of identifying a subject having an increased risk for developing psoriasis, the method comprising:
determining or having determined the presence or absence of an Interferon Induced Helicase C Domain 1 (IFIH1) missense variant nucleic acid molecule encoding an IFIH1 predicted loss-of-function polypeptide in a biological sample obtained from the subject;
wherein:
when the subject is IFIH1 reference, then the subject has an increased risk for developing psoriasis; and
when the subject is heterozygous or homozygous for the IFIH1 missense variant nucleic acid molecule encoding an IFIH1 predicted loss-of-function polypeptide, then the subject has a decreased risk for developing psoriasis.

48-69. (canceled)

70. A method of treating a subject having psoriasis, the method comprising administering a Tripartite Motif Containing 65 (TRIM65) inhibitor to the subject.

71. The method according to claim 70, wherein the psoriasis is guttate psoriasis, plaque psoriasis, inverse psoriasis, pustular psoriasis, or erythrodermic psoriasis.

72. The method according to claim 70, the method further comprising administering a type 1 interferon pathway inhibitor and/or an Interferon Induced Helicase C Domain 1 (IFIH1) inhibitor to the subject.

73. The method according to claim 70, wherein the TRIM65 inhibitor comprises an antisense nucleic acid molecule, a small interfering RNA (siRNA), or a short hairpin RNA (shRNA) that hybridizes to a TRIM65 nucleic acid molecule.

74-79. (canceled)

80. A method of treating a subject with a therapeutic agent that treats or inhibits psoriasis, wherein the subject has psoriasis, the method comprising the steps of:
determining whether the subject has a Tripartite Motif Containing 65 (TRIM65) missense variant nucleic acid molecule encoding a TRIM65 predicted loss-of-function polypeptide by:
obtaining or having obtained a biological sample from the subject; and
performing or having performed a sequence analysis on the biological sample to determine if the subject has a genotype comprising the TRIM65 missense variant nucleic acid molecule; and
administering or continuing to administer the therapeutic agent that treats or inhibits psoriasis in a standard dosage amount to a subject that is TRIM65 reference, and/or administering a TRIM65 inhibitor to the subject;
administering or continuing to administer the therapeutic agent that treats or inhibits psoriasis in an amount that is the same as or less than a standard dosage amount to a subject that is heterozygous for the TRIM65 missense variant nucleic acid molecule, and/or administering an TRIM65 inhibitor to the subject; or
administering or continuing to administer the therapeutic agent that treats or inhibits psoriasis in an amount that is the same as or less than a standard dosage amount to a subject that is homozygous for the TRIM65 missense variant nucleic acid molecule;
wherein the presence of a genotype having the TRIM65 missense variant nucleic acid molecule encoding the TRIM65 predicted loss-of-function polypeptide indicates the subject has a decreased risk of developing psoriasis.

81-86. (canceled)

87. A method of identifying a subject having an increased risk for developing psoriasis, the method comprising:
determining or having determined the presence or absence of a Tripartite Motif Containing 65 (TRIM65) missense variant nucleic acid molecule encoding a TRIM65 predicted loss-of-function polypeptide in a biological sample obtained from the subject;
wherein:
when the subject is TRIM65 reference, then the subject has an increased risk for developing psoriasis; and
when the subject is heterozygous or homozygous for the TRIM65 missense variant nucleic acid molecule encoding a TRIM65 predicted loss-of-function polypeptide, then the subject has a decreased risk for developing psoriasis.

88-98. (canceled)

99. The method according to claim 27, wherein the psoriasis comprises guttate psoriasis, plaque psoriasis, inverse psoriasis, pustular psoriasis, or erythrodermic psoriasis.

100. The method according to claim 7, wherein:
the type 1 interferon pathway inhibitor comprises an Adenosine Deaminase RNA Specific (ADAR) agonist or a DEAD-box polypeptide 58 (DDX58) inhibitor; and/or
the TRIM65 inhibitor comprises an antisense nucleic acid molecule, a small interfering RNA (siRNA), or a short hairpin RNA (shRNA) that hybridizes to a TRIM65 mRNA.

101. The method according to claim 30, wherein:
the type 1 interferon pathway inhibitor comprises an Adenosine Deaminase RNA Specific (ADAR) agonist or a DEAD-box polypeptide 58 (DDX58) inhibitor; and/or
the TRIM65 inhibitor comprises an antisense nucleic acid molecule, a small interfering RNA (siRNA), or a short hairpin RNA (shRNA) that hybridizes to a TRIM65 mRNA.

102. The method according to claim 72, wherein:
the type 1 interferon pathway inhibitor comprises an Adenosine Deaminase RNA Specific (ADAR) agonist or a DEAD-box polypeptide 58 (DDX58) inhibitor; and/or
the IFIH1 inhibitor comprises an antisense nucleic acid molecule, a small interfering RNA (siRNA), or a short hairpin RNA (shRNA) that hybridizes to an IFIH1 mRNA, or the IFIH1.

103. The method according to claim 1, the method further comprising administering a therapeutic agent that treats or inhibits psoriasis to the subject.

104. The method according to claim 103, wherein the therapeutic agent that treats or inhibits psoriasis comprises an anthralin, azaribine, colchicine, fluorouracil, methotrexate, methoxsalen, resorcinol, a retinoid, a corticosteroid, cyclosporin, iodochlorhydroxyquin, salicylic acid, vitamin D, dapsone, somatostatin, sulfur, a tar, zinc oxide, hydroxycarbamide, a fumarate, or ultra-violet light.

105. The method according to claim 70, the method further comprising administering a therapeutic agent that treats or inhibits psoriasis to the subject.

106. The method according to claim 105, wherein the therapeutic agent that treats or inhibits psoriasis comprises an anthralin, azaribine, colchicine, fluorouracil, methotrexate, methoxsalen, resorcinol, a retinoid, a corticosteroid, cyclosporin, iodochlorhydroxyquin, salicylic acid, vitamin D, dapsone, somatostatin, sulfur, a tar, zinc oxide, hydroxycarbamide, a fumarate, or ultra-violet light.

107. The method according to claim 27, wherein the therapeutic agent that treats or inhibits psoriasis comprises an anthralin, azaribine, colchicine, fluorouracil, methotrexate, methoxsalen, resorcinol, a retinoid, a corticosteroid, cyclosporin, iodochlorhydroxyquin, salicylic acid, vitamin D, dapsone, somatostatin, sulfur, a tar, zinc oxide, hydroxycarbamide, a fumarate, or ultra-violet light.


SEQUENCE LISTING (34 sequences)

SEQ ID NO: 1 - DNA, 51611 bp, Homo sapiens
  source 1..51611: mol_type = genomic DNA
caaactctgtaagaactgcctgacagaaagctggactcaaagctcctacccgagtgtgcagcaggatcgccccggtccgggaccccaggcgcacaccgcagagtccaaagtgccgcgcctgccggccgcacctgcctgccgcggccccgcgcgccgccccgctgcccacctgcccgcctgcccacctgcccaggtgcgagtgcagccccgcgcgccggcctgagagccctgtggacaacctcgtcattgtcaggcacagagcggtagaccctgcttctctaagtgggcagcggacagcggcacgcacatttcacctgtcccgcagacaacagcaccatctgcttgggagaaccctctcccttctctgagaaagaaagatgtcgaatgggtattccacagacgagaatttccgctatctcatctcgtgcttcagggccagggtgaaaatgtacatccaggtggagcctgtgctggactacctgacctttctgcctgcagaggtgaaggagcagattcagaggacagtcgccacctccgggaacatgcaggcagttgaactgctgctgagcaccttggagaagggagtctggcaccttggttggactcgggaattcgtggaggccctccggagaaccggcagccctctggccgcccgctacatgaaccctgagctcacggacttgccctctccatcgtttgagaacgctcatgatgaatatctccaactgctgaacctccttcagcccactctggtggacaagcttctagttagagacgtcttggataagtgcatggaggaggaactgttgacaattgaagacagaaaccgggtaggtgtctgttcagatggagctagccttttaggcagattttgcaaagcaggcttccttgtgcgtttgcccctttccaaatgtgcttgacctctttgggcaaatctagtttcttcctttgagaagacaacatctaggcactttaagacaattttaaaaatttaaacacaaaagaactacttttctaatgttttcactgaagtaaaaaggagttaactctagaatcgtaatgtgtattaacaggtaacttttattgaacacttactatgtgcagttggtatcattttcaaaatgtattactacagttcatgttcagagtaacccactgaggctcctaatgttattaactccattttacagatgagaaacctgaggcaggttaaataatttgtccaaggtctcccaggaaaaacagtggcagagctaagaatttagcttgtgatagtgttgaatgtttttcttatttaatgtagttccatggagcaggggttagtaaactacggcccatgggctaaatcaagcctgctactgtttttttcaggcccacaggctaagaatggtcttcagatttttcaatttttaattttcagtgttcataaagttttattggaacacagtcatgctcgtttgtttacatattgcttgtggctgcttttctactccaaaggcaattggatggagactgtgtgcccttaaagccaaaaatatttattatctggcattttagagaaaaagttttccaatctttgccctacattacactttgtgccatttagtcttagcccaaatgggtaaattgcaatcacacacacacacacacacacacacacacacacaccctttttctttaatcaaagtttcaacattctttgagaatcaaaatttagaaacaattcattgaaagaatgtgttgctagtttcagtttttcatgttagtgaagaaactcatttttcttaaggtagcattttgacagggcgaattttactgatgactttacattgaatgtgatatgaattattttttgataagtgaagatgtactgtgatgtgtataaatgctgacctgtttgaatgtgacttattcaggctcgtaagtcaaaagtcttgttttggaagacatgtagacaaataaagccatgactaaaagtcaaaggctttgtctacaccactctatgattttcacatggtttttttctctctctctcttttgggtcatccattgtttgtatattcatacaaacttctgggcatttgattgatttgctatagcctatgatgcaatttagtctttagtgaataaagttagtgatcttttgtagacttgacagacacagggcctgtccaaagacccaaacatgcatctaattttgatggccagttttcccaacaagtaaattactttgcactgttgtctgtaatgtttatgtcgttttgtgtattgagaaccagctagaacgcattatagtagtccagctggatatgacaaatgtgtacatcactgtggcagaatcttaatttggaagaaagtatgcagccttcaagccagctggagatggaagatggcatttccagccacagtagctgttagaagtccagaagcaacagagtccagtaacaccacaaagctgtttatgtttggtgggtatgatgtcccgattgccagcgaaacagcacttctccctatttactctaaatacttctgcctatcagcatcacttttgtcttgcctggattgaatttccgccagctagattttatccaagttcttgattctggcaagcttggaggcagctagcagtaatgcaagggatttgacacaaagaagatatagagtaacgggacttttgtgtactggtagcagttaaggccaaaacaacccaaattctccctctgattcacatttcaaaggaggccccaaaagactgagcagctataaatttgaggctctctaggaagcaatagtttctctgtgagagtgtggaataatttttgtgtgcatcatttacatgtattatcagttccaaaactaagatatggtggacaatgagacggaagcattaagtttggtagaataatctatattgaggtggtggtcattgacaaatgtttatcttacagcagcaaagtgtgataagactgtagaaggggaacacatggatttgaggtccatcttggcttcttactatatgcgttactttgggccaaattactgaagttccctgatgctacatttacctgtgaaaatggacatcataaaatttctcacagtattttagtcaacatcaaatcagaaatagtgtttatgaaaaagcttataaatcagagctatttgatatgataatggtgttgacttttgtaaccattcatctggttacatttctgtgtaaatttaagtttgagagattgaggtaaattccccatgtcgcaattgagatatacttcttaaagtccctagattatactgatttgaaaaagaaattacctctttcatcccagaacctaactaggattaggtagagggttggtgtttagtaagcattgattacttgattgatagactaattgaatttatttctgaaagtgtctggaataagcgttgaaaacacatgacgtattgaattgtgttgccaggcgttagagaatggatgaatgatgaaaataagatttccaaatttgcttttttgaaggcttaaaatagcatttaactaagtaatcaagacttctataaaatggtcttgtattttcattctttttctgtttccccatagacattttctacttgatctattattttaatttaggattcccaccccatttcctagcccctgcccgtaattacagcaggccttctgaaaatgactttatacataggtttgtgtccagcaagtacccaaaaacttattcattcattcactcattcattcagtttgaatttattgagtggcatctttgtgccggatgccatgctagtggtgaaaagataggagaaaaacacagagactgttttgtcctttcgtgaagttgtgttgtagtgttaattagtgttgataaaagaatgtctactgtaggccagtcatggtggcttacgcctgtaatcccagcactttgggaggccgaggagggtggctctcgaggtcaggagatggagaccatcctggctaacacggtaaaactccgtctctactaaaaatacaaaaaaattagctgggtgtggtggtgggagcctgtagtcccagttactcgggaggctgaggcaggagaatggcatgaacctgggaggcggagcttgcagtgagctgagatggcgccactgcactccaccctggtgacagagcgagactccatctaataagaaagagagaaagaaaaagaaagaaagaaagaaagaaagaaagaaagaaagaaagaaagaaagaaagaaagaaagaaagaaagaaaagaaagaaagaaagaaaggagggagggagggagggagggagggagggaaggaaggaagaaaggaaggaaggaaagaagaatgtctactgtatacaagggactattctaaatactagagtttcagcaataaatatatgccattttatcaatcattataataaagcaatccttaatggtaagggggtacccctgtactgccttgctcctcttttaggatgctaagttacaatttggcattgttacatcttagttataactaagttacaacttggcacacccatcaatttaggcagtcatgaactctgggaacgaaagtgatcatataaacatttccatagtgtcttaataatcaagctgaacagctaaagagagatgcatgtttcatccctggccagttcagaacacaatgcatctgtagtcctggagactccacacaccttccatttacacagaagaaccagagatgccatgactagtccacctaagaacagtccagttctcccaacatgaagttgagcacccaagtcaatctccccaggaacattgtattgtctggcaaattaggaaagataagaatttcatctgcaaaaaaaaaagtatgaataagaaattagctatgtcaatgtgacaataaatattcattgggttaagacattaactttttgtgggaaactggttctttaaaattcttttttaacatacaggttttgcctcctgatttacttttatctgagagttgaactctgctgggttaaaaaaatcaccattatatttaaatagctatctaaggttattataaagggaagaaaacattttcattaaactgtacaattgtgttggtaagccagtgctttatggaaatgtcattggtttcttcactgatttctgttggaaagaaaaaacatatcaaagaatttagaatatgaaagcattgagttgtattattaccaaattaattttatttcgtatttcataaattagttaacaatgtattaatagactacatagtgaaaacatccatccatttttaacttcacagagaaaacttcgagtgggggaggggcatgggtgagaattgttttatgacttgttgcagtctcaaatgtaaatattcacctcccacgaatttctgtaattgtgtttacatattgggatcaatatctttacaatgttttaggagaacatattaaatttttaaaattatataattgcttcaaagggagctgatgtactttccagcaccaaaactgagaagatttcttaaaaattcagcatcttgatgaggctgctaaaattgcacagatatatgtagttccacattcaaattgtaagaatatgtttaggaaataatgacatcatgatccttatatgcttggactcaataacggcaaagaaagcttgaaaaatatgattccacagtaaaagttgacttaatagaatagtggttgaagaaaattttgtggatagaatatgcttagggatttttaataacagcatttgttgcctcactggttctttttaaggaaatccttcctgattgcttcagtcaaatatatttaaagctgattaaaggatggggtaataaaatatgagaatggctgagttaaaagaagacactgaaaccagtcttatttacatctatagcaattacttgttaaattagcagatgataataaagatgttacaacatcagggaagccaaagaatttccattaggagactaagagagctagaaaaaaatggtaaaaaatgagcaaaaatctgcttagaaaatgttgtgggtgacatggactgaattcacttgaaatttactgagttcaaatagttatttttaaccacttcttgccagaaaatgaatttaaaattggagaaaattataagctataattatagttcaagaaggcaggattcagcctctaaattcaactgcctgggatcaaattctacttctgccacttactcactttgtgacctcagagaggtacctaaacatctctgtgcctcggatttctgatctgtaaatgagttaatattggtacctatttctgggctgctgggttaatatattcaagttccctagaacagcatgcttaactactagatgctaagacggaaatatcccttgggtagagaccatgctgtatgtgcttgtgcaggtgaggtgtttgtaacaataactcttcccaatcatccataatagttctaacactgaattagtgccagacattgttccaaaaatgttaacaaatttctgacataaaaggttatggaatatatttatagaactagaaaatacataaatttgtaggaaagaactaaacaatccaaatgatacatgtggaagattggcaaatgttgagaaggacctttgataatatcaaagtcaatatttcttatctgggaggctgatagaatcacatgagatgttttactttttctttaatatctgagttcctaacatagacaaaataaatcataatcttttaggggtgtttcctagacatcagtattttttaaggctttacggttgtttctaacatgcagctaagatatagaaactctgaaatttcttttcagctgccttgtaaccacatattgtaaccaaggtctagggagatgaagtaaattgcctaatgccacagaaagctaatgatgtcctgcctaacttctttcatgtctaaacatggcttgagttactctttcctccaaatccttattgctatattaggacctagggaaatgcactgtgacaatgattacacaaaagatccacatttaaaagaaatttactaatctaaaagagaaaataaaagaaggctaaagactctaatgaatggatataaaaataattctgccccatatttccaaaaaaaagatataacaataaatttcccataacattaaaaaaacacaaatttgagaaaaattgattatagtaataatcaactatgcaaatattacataggaaaaaatataaatctgcagtagtaattacttatttcataatgataatcatgaagaaaggagataagaaatgtttgatttcccatttacagactccatagattgaaaaaatatagccagccctagatatacagaaagaaattggtgaaaatagacataacatggctgcagactaaagatactttctcttctatcctttattcaggaatggctgaggtaaaatttattttctaggtgtcaggtttgaataattcaatagcttttaaaaaaaatggttcatatttaaaaggacacttggtccagagatttttgttgttgttgttgttgttgtcccggagatttttttccctgccgacttcagaattgctacaggtaacattgctattttcttcaatgtttcaatgtaagcctttatttatacttatgcttaattgctcagtttttctaaagggtatttcctgtttaaggttattcagaagatgtttgatcttacttaattctaattttctcttacaacagattgctgctgcagaaaacaatggaaatgaatcaggtgtaagagagctactaaaaaggattgtgcagaaagaaaactggttctctgcatttctgaatgttcttcgtcaaacaggaaacaatgaacttgtccaagagttaacaggctctgattgctcagaaagcaatgcaggtatttgtaattttactgaggaagattcttcaaattctgcctagtgattgagaattcaataaagcagaaagacacaatgctaaacaccaagatttttagaatcaaaattttaagagtggaacatatgtgatagatcatctattttatcaccttaacaaatgaggaaattgatgcctgggaaaatatcaactgatcaaagaccatttttcatccaaccaacactctttctactaccttattttaaaaacaatatgactaccagaaattttatcaagattatgtaacattaaatatacacaaatacattggaagaaagtattttagtggttataaaaaatctgctgcatattcttctgaacatttttatacattttaagcaatgtattaagcatttgataaaaggattgttttgtcaatatatctcaaccttacctaatgtgagattttaaacagcattcaataatagtaataacataacattttatagctccttctcctacattatttgtaaatctcacttaaactgctatcttatttttcttttcgtagtatgcatcctatgtttccaaaaatatcatatgaaagacaaattgaaagttaactattaattaattataatcattgcagttttgttctggtgaccctaactaatgaaaactttgctgtaaaaagtggggtgctgctgtaacaaacacctaaacgtgtggaagtggctttggaactgggtaattgatagagactgtaagagttttgggtgaatggtagaaatatgggtgttcagggtgattctggtgaggtcacagagagaaatgagaaaaatgtcattggaaactggaagaaaggtgatccttgttataaaatgggataaaacttgatctcacaagtaatggatttggatatttggctgagaaaatttcttagcaaaatgctgaaggagcagcttgatttcccctaactgcttatagtaaaatgcaaaagaagagaaattaattgaaggagttgttatgcaaaaaggaaccaaaatttaaatatctggaaaattctcagcctatccatatggcaaaaaatgagaaattgtgttataaagagaacaaccaattgataacaagatcatgggtgctactgatggacttaatcagccatttcagcagaaggtaggagtagagatatattataccagcaaatacacttccagtttgaactgaaggagacagagaaagcaggacagaatgagaaaaggatactggactttctggattctacagaaccagaccatagagctatttggctgtgaatgtgtactattcttcagggaaaggaaagaatgcccccaaaagtgatacagaagtcatcagggttgccacttccaccacagacccagaggccaaggcagtttcctttttggtttcaaagggcaaaactgtctctctggttttagtgagccctcaggatgaccctgcctagtgtctcagaagcagagctatcctcacagaaagctgtgtggatggagcccttagagagagcaggctgtcttgcagagccctagaacctgccattccgtggacctggaaggcagagcatcaaaccaaacagagctattctcaagccttataagccttaagatgtgacagaatttaccttaataagttttggatttgcttgggacccatcacccttgctttctttcctatatctcctttttggaataagacgtccatcctatgcctgtcccaccattgtattatggaaaagaaacatgtagctcaactggtttcacaagttcacagctggtgatgaatttgccttatcatgaatcacacctctagtctcatctacgtctgatttggacaatgtttaggtgagacttttgactttagacttagagttcatgcttcagtgagttaaggcacttgggctgatgagatgagatgaatgtattttgtatgtagtaaagatatgaattttgggaagccaatggtaaaatgctacagatggaattttgtcctcctaatactcatatgttggaacctaatatgactctatctggagacagtgatagtaggagatagttaagattaaatgagccaggccaagcgtggtggctcacgcctgtaatcccagcactttgggaggccaagacgggtggatcacctgaggtcaggagtttgagaccagcctggccaacatggcgaaaccgcatctctactaagaatacaaaaattagctggggtggtggcatgcagctataatcccagctacttggaaggctgaggcagaagaatcacttgaaccaggaggctgaggttgcagtgagccgagatcatgccactgcactctggcctgggtgtcagagcaagactccgtctcaaaaaaaaaaaaaaaagattaaatgaatcataagaatggggccttaatctgatagaattgtggccttttaagaagaggaagaaagagaaaaagagaaacatctgtctctttctctctcttaaccgtgtgaggacgcagggagaacgtggccatctgcaaatcaaggagagagtcctcatcagacattgaccctgctgtcaccttgatcttggacttccagtgcccagaactgtgagaaaagaagtgtctgttgtttcagccactcagttgatggtgttttattatggcagcataaacagactactgtaggagcttagactctgggtcctgatacatatcggatcctgatgcttatcaaatccaggctccttccttgaatgtgtgacctttggccaggttctcgacttctctatgcctcagtttactcctcagtaaaatggagataataatattacttatcacctcagtaaaatggagacaataatattacttaccatctaggtttattttgagacttaaataacttaatgtatgtaaagtatttatattgtgagtgcaaaataaattaactattataatagtccctaatgtacttaatatctaactgcaaacattgccaagttttcttcttgtccaaagtcacaacagttttctttaggaaattttccatacaaagtggggcacattttcactgggttctttaagatgagataataaaaaatgatacgttagaaaattttccttatggcatcagtcttctctgacagattgcagcttcataaaggcaaaaactcttgattttctttttaacccacataagcccttaataaaatacccattacatgtagttgaagtaagaaaagctggaagtgaggaatggaggaaagaagaaaagaggggcacaaaatatttcattaaatgttttaaaaatccccagaaattcagaatacatgaatataggctgacattaaagcagatgtacgtctcgtgcagaacacacagtgaggaaacaaaatggcaagagcaaagctaaagaaaactaccaaatttgcattttcattaattcagccttaaatgtaactttgtgtcactgaataagtgacttacttcctaaacatgacggtcagacaggaaatcacttttcttgtgattagcataatgcttatattgtttttttagctacagtctaatcattacaaagaactactcgttgtgtccatattaaaattaaaagtaccttgccataacaaaagcacattctaagcctttcgaaatgtcaacatattaaaatatattaaagcaatctctattgcgggctctccattttgttttcaaaataacaataacagctatttatattgtataaaatcttgcattgcaatacaatttacattctattcagaacttcaaagtatttaaatatattcaggcaatctctattaggaacctctagtttgttttcaaaataacaataacagttatgtatgctctacaaaaacttgtatggcactatgatttgcattctaatactgttattcacagagattgagaatttatcacaagttgatggtcctcaagtggaagagcaacttctttcaaccacagttcagccaaatctggagaaggaggtctggggcatggagaataactcatcagaatcatcttttgcagattcttctgtagtttcaggtacttgaaacacatacgtactttaatacagtaattactacttccagaaaaaccaactgggcagaacctattaatcagagggagaaaatgtgggcattaaggttagtaaacatttatattaaaataatatttttagaggagattaaattaaaataattcttggccaaaatgctttctaaaactaatccaatatcgatgtgttataaatatgaacacaattgcatctaattttaatttataagaaatccatagtcattgtaagataagtataagaatgccttatatttcctggttttcaaagtacttataaatagatcagtcaatttaattcacccaaaaccctgaaaataaaatagattcattgctttttttctctggtttaaactgggatggctcagagaggttcaactgtgtcgtaaaaattaaaatcaaagctataaaaaggaagatgaacagatgctgaactagggttggcttgaattgcattaactaatcggtgaggactgaagactaccatgtccacctttttgatactgaaacagccctggcagaaacaaattatttaattattttttccctttctaatctgctcgtgataggagaatccacctctccccttctctgccaaaaggacttattctgttttcacaagaaatagttgcttaaagcctgtggtgtgctggtgaatgtaacaagtaactctggggaaaagaaaaagccctagtttgtagcatttgctagtctctgtggtttaaatattctcattcaggcttatttcaagcttccaacataagatcactgaacacaaagctgggaagagctgtgcagtggcacatctttatacagcattcccaccgtacagatgcaaaagacggagatagcctccagatcaaagatgatagttaaatgattaggtgatgagttttaaatacttacaacctttgcctgaaaataatttcttttatggtaagtttatacaatttaatttttgtaatggctgtggttaaaaactcagaaaattcctgaaaagttaccagttagctcctgtgagcccatataaactaactctagcacaccactgcaaagtttattatttttatttttttaatttcattttattttattttattttatttttggagatggaatctcactcagtcacacaggctggagtgcagtggcatgctcttggctcactgcatccttcacctcccgggttcaagtgattctcctgcctcagcctcccaagtagctgggattacaggcacccaccaccatgcccggctaatttttgtatttttagtagagacagggtttcaccatgttggccaggattgtcttgaactcctgacctcaggtgatcccccccactcggcctcccaaagtgctgggattacaggcatgagccactccgcctggccttgttattttgtacttagataagtaatatgcatgcattgcaagaaatctgtaatacacagaaacacacaaagaagtcaccaatattcccagaatgtggatattacacttaacatttgggtatgtgtgcttctgggctttttacatacctgaatgtgtatatatagtgtcatttctcttatgttcattaaatattcttctacagcctggtttttaatgactaaataatggtcccaccatttggatgcagtatgattaattgaccaattgttgaacatttaaataatgcctcgatagctctctttgtacttatgtcacagtttgctttttaaaaacatattatataattggaatttctgggtcaaatgatttatactattttaacacatttgatacttaatgtcaaatctcactccaggaacttgtaccaatttacatgttctccaagagtatgggatatgagtgcttatttccctgaattctcttctacgagaatgaataatattatcatgttcaatcttttcaatttggctgctattaaaaatggtatttcgttatttgaatttacattttcatatttctgctgaaaaagaatttcacaaattcattctctttgttttgttaattatcaatacttttaaaattcacttttctattagggtattcatcttctttttgctgacttatatgaactatctaagtatataagatatcaattcattgtcataaatagtaccaatttttttcacagttggcttgctttttagcctgtttgtggatttttctctttttctttttttgggggttctgggggcaggcggggacagggtcttgctctgtctctctgtcgcccaggctggagtacagtggcacaaacacagctcactgcagccttgacctcccaggcgcaagtgatcctcccacctcagtctcccaagtagctggcatcacgggcgtgcgccaccatacccagctaatttttgtattttttttagagaatggggtttcaccatgttgtcaagacttgtctggaaactcctgggctcaaggaatcctcctgccttggcctcccaaagtgctgggattataggcttgagctgctgtttgttttttaatgtaatgcgattccacctttctcataaactctggtgctgctgctgctgctaatagcaaagcttatacaggatagttttttttgttgttgttgttgtttttcatttgagcagctgcaccttcctttagaaatcatgattctataggaatgccaaaattcactatctgagtgtggagttttcatgttttaataaatgtcctttacaagctactcgacatctctatgagttactggccattttctgtaaatccacactagaaagaagggaagatggagctactgctgagagatgaccctgcatcttaaatgagggtttatcatcaaagattaccagactaatgcattgcattagcttgtagttaatctgctctgaacttctagtgaacaaaattaaaggcaaagttttaaatttttttaaatttttacttcttagtataaaacttaaagatgtatcttgtgaaaaaaattttaataatgatattaggggacagaaaagctttttatttttttttttcctcttgggttccttcctctctatctccctccttcccttcccttcctttcaccctaacttccttccctctcttcctttcacttctttcatttaacaactattttccaaactcctatcatgtgccaggcaccttgttgtatactgtgaactcaggaacataacagaaatggcttcatggccatggagcctaatatgactctatctggagattgtgatggtaagagataattaagagtaaatgagtcaggccaggcgtggtggctcacacctgtaatcccaggactttgggaggacgaggcgggtggatcacctgaggccaggagttcaagaccagcctgaccaccatggtgaagccctatctgtattaaaaatacaaaaacttagccgagcatggtggcagatgcctgtaatcccagctacttggaaggctgaggcaggggaatcacttgaacccaagaggcagaggttgcagtgagccaagattgcaccattgcactccagcctgagcaataagaacaaaaaaactccgtctcaaaaaaaaagaaatgacttcagcccccatggagctttccactgagggaggtgtcggcccagcagtagtaactgtatagctacacttttagtaagtgccatgaaagagtagtacagagtattaaaatatataatagtctgagagcatgtaagagtatacagtaaactgcacagtaatccgaggatccatccaattcacaaacatcttaatggtcttctagtccataggttctttatgatatctggatttctcatgtggatttgaaagcaggttagtttaatccattctggccctttccagagcactagcaaagagtcacttatttatgaaaatgtagtgtgtttgcttcagacctttgctttagactgctgctgtcattccaggtatctgtttgtttccttgttatcttaaggactaagaattactgatattaataacttataaagtaccattagtccagagaaattattgagttcttttgattgaagtttttgttgcagaatttactatggaagggttttgcctgcataggcattttctgacagaagtaacaatcaaggtaaatgtgaaagtgctccatagcttctaatatgcatttgtgtagttttgttttgtgtatcttcttgatattgataatgtttagtctttattttgaattccattatattaggttaaagtttcaaatagttgccaagatagcactgaaaaaaacttgtcacaaagtctgtaaatatgatttttatgtgtataaagaaaagactcaaattatttgtaattttaaattaagatatttataattattatttctgaaccagtttatcaaatattttctcagtgcatagtagtcttagtgcatgcaaatattaattatttatagctttaatgcaaagattatgtgtgtgtgtgtgttttagcaccttttaccccttaacacatctgatggataagacacactaccatcagtaacagtggcccaccttgaaactaattttaaacacatatccctttggcactagaggtgacatatcagagttctctagtggattgttctagagagtgatttcatcatatctccaaacttgcgggttgatatgtgtcctttttcctcctcccacctatgtttacaaatcaattgcttctcattaaaaaagtggttagtaattgatatatcaattttttatttgcccttttccaaattaccaaaacaatttctaaacaaaatttgaacagttctcagactattaatggcaatttaatgctggatgctatagttctgctttctgaagtcactggtcgtcttataacaaaagataactgcaaaactttggtgtttctctagatcagatttccctaggaaacatcagagagacactaagcctgctttcattttctattaaaagcattttaattatagcaaataattgacttctatatagcaaatattttacttctaaaagcgaacagttaaattagttgtgcagtcagtctttgtctgctactgctgctttgtcaaaacaagtgaatagttacttcacattaaccttttttcattatactattttaggaattaatgcattacaataaaatcaaaggaaattaagtgacatttaaaactaagtcaactttattgcatttggtataaacttttatgcagaatggagcagaaaatccccccaaacttctttaatcattgtgttacagctgccctattgtgaaaactagaaaatgcaaatgcctcagattggccagcagagggcgcctttacatttataaagccaatggaatttcactgtttcagcgtgaaagattataatgtaactgtagagtgtgccatgtgtgaatatacagtgcgcatgactgtgattgcatgtgtatattttgatttatatattcgttcacgtgaagattttaaaattccaccccagctaatgagcttaccaacctgcttgttttcagactttaataccttctttctcatatttccccatatgttccactccttaggtttatccggtgacattatgctttggaatatattgtggatatattttgcatagatcatgttctgtaggaggggtttaaaaattgacctttacaaaacagccttcttctccctggtccaatctgttatagcactttttttgttgttgttttgttttgcctttttatagactaaagtccaatcttagatttggtctatactaatttctgtaaaagtgccacaaaatttatccttgcacttgaggaccagaataatgttctgtagcactaacctctagaaatctagagatttttagaatctctagaaataaaaccatagcatttgaaacctaagtggaaaaaaataattgatacagtagtttacaataagactgtttcagaatcattcatgctatatcaggactggtttgaagtaagagcactcctttactttttaatttttctaaacttagttatcattaattattgagaactagcagtactactgaggcaatccaaaagacacatattaatttgagctgagagaaagagagagatgctgatgctgacactgatattggtacttgttaaaggattatgtaagtttgtattttatttgaaacctagcatgaggtatctgggtcagttatatggcttacagttgcataatagtcctaggaggattaccattcatacctcaggaatggggacataaatattatgacagaaagacagaaagaaatctccccagtgtgcagttaatacatgttggatttattttcatgatatctgtttatgactacaaacataaagctgtttttttagcagagttttaacaaaagcagtgattttaattttgactatcagatcttcccttcacaattttaggatgtgaatgaggtcagaagccagagaaataggaagaaggaggtgagttggtcagataatacagtatcattgagagaaccaagcactgaggaattgcgttcactctcaaaaagtttatactctaatgaaggaaaacaagtgacatgcctccacaaagaaactgtaatctagccatggtaaaagagatgcaagatctatattacaggatgaagagagaagatctgtaaaagaaggaagactgacgagagaaagcttaccagatgaggaatatcagatatttcatctgagccttgagagcactatgctggatttgtaaatagaagtggcaggcaacatttcaggtttcagaggcatcatgagcaaaagcacaagcacaagagagctcaaaatgtgttcagaggaacagtctgatgagagaggagcatttgtggaggagggcggtaggatttaagacttctaagatcagtgaaagttaacgagtttctctaacagtaatttttacaatgctttttctcacacacacacacttttaaaagtacaccatttatcatttggataattatacaatgccagcacaatgcatgtgtgtacaagcatcatctcagatgatgattgtaggaaagtccttaggtagaggaatatatctagcagagcgctggaactggatcttactggcttgtaagaccccagtgtgcacatctctttccagctccacacaggacatcatgctggaaattggccacggtgtgaatatttacaccatggaaattgacaaaccttttaaaaatcctgtctccccccaccaaagggccagttgttgaacatttacctgggcacccttagtgtattcgtatctccctccattctttcatgtctgcttaagtttgcttaactcctacagcctttcctgaaaagtgcttaccagccaaagaggtttctaattcacattctaccttgccgtctccaaaccttgttattgccaagatttacttcccatgtggttacctggcttctcactattaactgactctactctaggactttttgcctccattactgactggcagtgaatgaatgggtcattctggtgtgtgttgttatccttgcaacctgtgctttaataactacctctctttattagaacctaagtgcaaaggctaaagggcatagatgatacaatctaagccaacctcattacacacaattcaaatttaattctgttagaaacatttcagcagtagaaattctataaacaggagcagaggagctccatggatagccatgtgggcctgtggctctaggagagtgatttttctcgttggggaactcatttcctagaagcttggatccaggagagaatatgcaaggtgggtggaggtggcttctctccattctgtgggaacacatgttccctggcacggagaggaaaatctgacagaccaaaatgtgagtgtgtgtgtgtgtgtgtgtgcgcgcgcgcgtgcgtgtgtgtgtagatccatatacatagagctgaaaataaaagtgatctatttcctctcaaaaaagattaagttatttcaaaaggaaaggaaatatatattattagcagtatgagtgtaagcccacttgagaagcccatgcagtctttgtgtgtgtgcccaaacgtgcacacatgtttcaagctttcggggtagcttggaccttccaggcgtgaggattatgaaactggagggaaggtggggatagctgcaaaggtgaagctaaaatgcatcacaaggtcagaactagaataaacatgaaattaccatgaaaagtgaaattggactaaagaccttgtcaccactgactcgcaggagaacaggagcagttggtgctgttctttctcaatatgcacagaggccctttcccctcgcgtttcctgctgtaatgcaaaaccacttttcgggaaaaatagtgtgtggcagactgaagccaggggaaagaaacgaaagatattctgtggggtgataaatgcctctaggccaaaaatagaagaggtactacctggtaggaatagaagcaagaatcaatttgaattttacctaatggagcttagctctttgaataaagaatgcctacttgtccactacttgccatgttagctttttccagcagagggcagactttacattgaaatagatctaaaaaggccttcaacctcttgctttatttatttattttttttggtttgaatattttaaagttcaatatttttggtttctaaatgatttaaaaatattttaaagtatttaaggtctagagtatgttaaataagaaatttcttggatttcagttaatatttctgccttattagcaaaatactcaagatgtgattgcaaaataaggctttttttttccatggcttttgggctggtcttaggcacttgctgtcattgtaggcaaggctgttcttttttgtgttgtttacaaaattatgaaagaaaatcctatctcttaactggatggttataattttcattcattccaatagcttttgatataaataactgcttttctttaacagttcaattactaccatcttctaatctgtttcatagttagctttgatattttccctagaggaaatgacacctatataaaacttaactaaacttattatacaatataacttcaatattcaaagcccttttaatgagaatttaaaatttgtttctcttcgagtttcattttcttggtctaaccgaatcattctcacaataagtgagaaaatagagactactatctctctctatatattttttaaaacagaaggttaatttgcttgtaagtcgtggctacgttcgatctggagacttatctgttggactccaaagcacaaatttttttcatagcaaggcctgcctccaattaactccatgcgatctacttataaaatcataaacgcaggactttattcattaggacttaaatccagactgacatagtttgaaaaatacaaagaacaaaacaagataaacctaaataaatcaatttcagaaattcttggcatttaaactcaataggtcggtttaattttggcttcacgcaaacactttcttgatctcacttttttgggagtgtgtgtgtgtttgtgtgtaatacggtttttaaagtgaatgcaggaggaagatctctggggaaggcattctccctattataagaaatacaatatattttcttttattattaaattttacttatatttctatgaaagcaaaaacgtatatcaaaatgcataagaatttgctgcctaggaaacaaaattatttttatgtggagaaactgaagatttatatacccttaaaatgacaaagaaatcgagctccatatattcactttcattttcataataatctctacaggattactgcaattaattttatgtaacgagaacagaggtgttctttttatttgagtgcagaaaaaagtttaaaaagaggtgtggacacttaaatgtaaagttagtgaaaatctatcaatgaaaccagaaatgttagcaaagcaaaatcatctctctaagttcttttcttggaaagataagctctgtttcaagatggatagaaatgtgacaatacaatgaaaatgtctcttgctgttgaaaaacaccaccaccacagctcttaattcagttaacactgtgtttacaaaacttcacccaaatcatcagccatgcttagtgttagcggaaaaagcactggaacactggacttggattcagaaatgaggattctgccccactgtagctgtgtgatctggatatatcagttaacctctcagcgccttggtttctcacctatgaaacaggagataatatgtatctaccacacagaatgttgagagattaaaatgaaatttacattacatctgtgaaaactctgagttcagtgtcttcctgacacattggtgattggtttgttagatcaaaataatccagataatagactttattaataataccctattttgggtagcaacttttaaaacaattcatcaattattcgtgcacattgcataaatagctccctcccaaaacttacggatgttagaacaaagcttgggagaaatcttgactctacatgtagatgaagtaggtttaagattaaagtttaactgctagtgattagaaaatgaagtggaaatcaaatataattttcatttttattatacttggaaaatttaatgatacagtcttagattttagatatttgttagtattttttcagtaattttggtattctcttcagttctcaatattacatttaaaacagcaaatgaaaagcatgtcaaaattcacttttttgcctcaaaaatcttagcctatatttctagatctagaattactcttgcaatgacattttccccatctcattttacaccttgccttttccttacaaatcatattctcttttcatcagtacatctgcccaaaacgctcttctacttgacagttctatcccacattcgcctctgagcttctaacaagacatacctctgggaattgcgaggtgccatattttaaatctctcagtgagatatacaggttttcccgtgtgatcataagggattcctcaaatctgagttttagaactaatctgcgacaactgataagcacagtattttcacattagtatatttatgttgtgctaatatacttaaacagtatgtatgtccattgagaatttttctacaaatttcttatcagcttacttttaatttttaagaaagataccttacatatttgattggcattggctgtaattctgttatagctcttaaactaaataggctcatccaaattggatgggagatgtaaatgtcacagattctcagtggaatactagtcagcaataaaaaggaacaaattagtaatacacacaacaacgtagatgaatcacaattactataaactaagttttaaaagacagacataaaggctacatattgcatgaatccattcatatgacatcctggaaaaggaaaaactattggaacagaaaccagatcagtggttgccactggttgtctgatgtggaacagaaaccagattagataaccagagggatttactacaaagaggcttgaggagacttttggggtgctggaaataccttaactgtgatggtagttgcgtaactgtatacatatgtcaacctcttcaaagttgcaaaactgtatacatttgtcaaacttctcaaactataaacataaaaagtaatctttttagtaagtaaattatatcttaatatagctgacttgaaaaaattaactacttaaggaaaaaggaaatatttgcagttttgagttgtatatcatctcttttcagagaagttttattaaaacatactatgtgtctattttttcatccatttttatttccagccataaatatgacactcagactagttttaaatagctaagagctacctcattggtgtgttaagaggctttaggatgaaatattatctaagtataaatattataaaatatttttaattaaaagaagaacattatctaatggaactcctatcctaagaatatatcatttcttgatttttatttcattttctaattgccagcaattaaagtcctgtgccaagagcttgttgtacctaatattacttaaagattatcaagggctgtgaagtaagtgggattagcctcactttaaaacaaggggaacttgttaggttcagagcaaagtgaatagcaaattttgcaagtccttctgattccaaagccaatattagtaaccaccattgctttatttttaccagtttaacgatagttctggtgcttgaggatgaggttgtgtaagtgtttggaaataaactgtaagaaaagaattcttctctgagggaaagaaggttttcaaaaagaattgatcctgagttctgaaaacagaatgtcaggattcataaatgaaggatgtgcactgctattcatccatattaaccataactctttgatagaggcttaatctaattatagtaaagaaaaacagttatttccaacctcacatgagcttctagtacaactgtgatgccaggctttagtgatattctttttgaacaaattctctttgagattttacatctatgtttatcaccaaactatcattttcataactgatcagtagatccaggtactttattaatttttgtactttattaattccctatcattttaaatgaaaagacctcaaaggtttctatgtattattttttgtttaaaagacatatcaaacctaaatagtgttttcttgggcaacatttacatgtagaagggcaataaaagttataaacccttaggaatttgctttagctctcatactggatgtgtagtactttaagtaggtaatatgtatttgaatcactacccagcagctgctcatccatagtggatttttcttaatgtagcctgccagaagcgcttaccttttattcataattgtctgggtgtgccttccccacttgaattgaactgtatgtactgtgtgctgtagaggtgtgcagttaaaatacgtttattctcagaaatattttaaaaatattttaaaatgttttcctttgcagaatcagacacaagtttggcagaaggaagtgtcagctgcttagatgaaagtcttggacataacagcaacatgggcagtgattcaggcaccatgggaagtgattcaggtactgcctaactgttgtgtaaaccttaaaaagtgtgaaataagacgccatatagtggaagcatgggggctgttcataccctccctaatgtatttatgcaattaaagcaattcttttctacagtgaaatagttttggaactttgccattcactttctttaaaagtgtaacttacaaactcttagaaccaaactctctaattttgatccagtacatatctttctggtctggcatatgccacattccatagagtcatgtcatctattttatttatgggatctgtttcaattaatttgactgcatttaaatagactgtattcaatttggtacatttatttacttccaaattctactttttaaattaaaatactttttaaataaaaaattgatactatcaataagagaagtatgactaactagaaattacatatgtaagtaaaagaagtcaagtttgtataagtatcttttgaataggagtagagttggcaaaatatttcggtcactaaatgaaaattttaccaggtttttcccaaattactttcaatatcagttgtatattccaccagcaataaatgagagtgcctcttcctaattccccacaccttaattattacttcctgttatcagttttccaatttgttagtctgatagatttcaaattgtttctttcttttctttgagtgtatatttccatgatgactactaagctaaaatgttaacattattaaatgtaggtgatgggttagtgggtttctattacatacctttctatatttgcctacctgtttgaagtatttcacaattaaaatgaacaaatcacaaacaaaaataaaatgctttgcaaggggccagctaagaagcattggaaaaatgataaattaagaaacacattactttattcttcatttgtgattgagttgtttcccctaagaatatattatataattcagattatcctgcattgtcagaagaattataaaaattgatatccacaagaggcctaaggtggcaaaatagtttatagaaagattgtcatcaatcctaaatgctttccaaattagattgtagcacctcttttttcatatatccttttatcacaatgacactcctgaatcccttagaacaaattagtttgaggaaacccaattcaccaaatgtcattcacaactttaatcttcatttaaaagggtgaattcattttctgttcaattcagtcagtgtttattgagttcttgccatgtgcttatttctgtggattatatttgttttttctatccttgtcataaaattgttcaatttaataattaacatttctattacatgctatgctagaactcaaaatagataatctagctaatatttaacatcattataggttgttttaagtttttggagatcatgctaattttcatttgtctaagtaagggcttttcaataatatccattcattcaataaatatttaatgtgtaccttctatgtgcagggcatcttgcctatttagagctgttaacaaatgtatttgctgatttttagtcctcaatgtggcatgaaacacatgataagtctgaacgcaatctacttttgaaaagcaaatacattttcaaaggcaaatacatatttgaagacaaacacatcagaacgaacaccacttgaagttctagatggatcagtacttaggagcaaagaattactttgcctagtgggtagtcaatttccgaaatggtaaaattatgaaattccttgcccacaagtgcctggtataagctaaaatagaaaccaattcaaaaggaggtttagataaattggtgaactgttaaacacatattgaggcactgaaaagtattatagaatttgcaattgtgttttaatctttgaagttgacatcaaaagggacttcattctatatgtccttcctcctcctccagaggtcatatatgggccataaggtgtgacccaaaacaatcatattatattcctttgtggaagtgttatgccatagtagacatgattaccggtacttttaaagtataaatattctaattatattatgagaggcttttttttttctgtttcccattttcagaaagatatattgcctctcaaaaagtggcaatgtttttggcaatacatattggcctgatatgtagaaagtgtttccttactatttcaagataatgcctattaatatttgtccagctggaatggtgtatttttctgtggttacataccataggtaaaggaatactttgtagatttcaaacacatgcagtgacagaccttttctctccaagaataactcagctgtcaaaatcaatgtgagaatgaaatttgccacatggtgtagttccaatagaatattattatttaagccttatttgattttcttttttgaccatttatatggtagaatataattttaaggtctttaatcaatacttgttgtagagggagggcacagtgttattcaaagcttgtgcatattttaactgctattttcatttaattgattatgcttttcagagaaagccaaaaacaaacaaacaacaaacaatcaaaaacccaaacatttcttttataaaggaaattggctcctggctcattgataccagcccagttctctccttctgctgacacagaagagaaaactctcttttcattgcttctgtggataaattcttccatccctgtgtcagaggatgtgataaggcaagaatcaggctgaatatttagcactcacccagcttttattctttggatgctagtctcacattgaatccaactctaattttttcctgtatccacactgtgcttctttttaaacttagggccacctatcaaatctcctagtcttaaatcaaaaatatggaagattattgagctatctgtctaaccaggtatttttgtgtgcataacagcggtatagtgtcctagactctaaagtccttcaggacaacaactatattattgagagtgcctggcatataataggtgtccagtcaatattgactgaagaataaatttgtggtacatgcctacttaagtagcagtaaaacctcatgacttcagttctgaagttaaattgtaaacacaatagtccttttaacattctcgctcttcttaaatgtatcagcttaataaatgattagaatgcatgagtaatattaatattttactcatttgatttggttctggattctcgggaaagagacttaggaaattgctatatctttctctttgtaatatttcttattcaaggatatagtagtaagcatgaaaaatgggggaaattattattaaaatgtactcttttaatagtttattctgcaactattatgaaggaaggagcattttattagtattcttaaaatctacgcaaaaatgaaactcagatgaaattaaacaaagaggatttattcctatattttaacaaacttcctagatcaaacaaaaatttagcagtagtaaatttctgggtctttgtttagacactttataatgcatcttcctttgcaaatatactggctcttttggaaaagttagatgacaaactatcctaatagaggtgaaatatcatggttttgatagtctatgaaagctacagagggctaacattgttcttaaataaatcagagccttacataaatataatacaagatgaactattgtaaatattgtagagttcaaaacaagcatggaaacttataaaggtatattttaaatgctggtgattttaaatgagtttttcataaaaatcatattgaatgaatcagcagatgatccaacgatagcttaaatgtattgcatctgtttttactcatccatacaaaaggcaaatttcctgccaggttagtagatgatcctaagtggtatttggtgtgaattttcctacctgttcagatagtactatcacaacaagttaaaactcactcaaatagaattatccaggggctgaaatggagccatcagcaagttttttgaagttaatgggattatgttagtttagttttattgcaaacattgataagaaaaagaagttatttcatgtgtgatgaaatatggataaatactgttatttcattttttcttcttattatataagtcataaaatgggcttttaaattggtataatatttatgttacttcatttcacattataagctatgcattttttgatctttttcccctggtaatataggtcaatatggtcacttctaaataaaatatattctatctaaaacaagacatattataataattttattttaatatacataaagtttatgttagaacttattttatcagtattttaaatttaaaatccatgttaattgaaaatatttttaatatattatactttggattttagaaaaagttggaatatgtggagccataaacataaggaagtaataaagataacaattcacaaataaatggcaaaagtagtcatgatttagtttaaaccaacaaaattatttttacctttcaaggaaaacctaccaattaaaggctagcataggaagagttttttcctctactctctaaataacacttctgtacaacgtagttgcctcagatggaaaaaggaacttgagatagatagggtgtctgtgtgtgtgtgtgtgtgtgtgtgtgtgtgtgtgtgtgtgctttttggtatttggttgtcagataaagtttctacaaacgaaagaaaatagtatcagccataataccttctttgcaattaaaaaaaggggaaatacggaattgggaggactaatgattccaactttctcagctcctcttgattttgtaggtggaagggctaagtgaaacaagcagggcatagtggctccacaaagaaatgggaagtctgatgccttaacaactagacctgccaatcccgcaccacagccctttctacaaattcgccttggcttcacccactctgtgaaaacactcccccatgttgctccttccagtcctcttaattagttgactccctctgcacccccacacccccagactccttctgtgagagggagcctgtgttgtattctgcagatgcagagagcagacattaattggtacagaaaagagaaaatgaattggtttggaccacatgtgaaaaggagtttaaagcacaagaggcctacgttcagtttcaggcttcctaagctctgttattttgttgtcccttctcttatttttcttttcaaccttcttcagatgaagagaatgtggcagcaagagcatccccggagccagaactccagctcaggccttaccaaatggaagttgcccagccagccttggaagggaagaatatcatcatctgcctccctacagggagtggaaaaaccagagtggctgtttacattgccaaggatcacttagacaagaagaaaaaagcatctgagcctggaaaagttatagttcttgtcaataaggtattcaaagaacacataacactagttgatcattttcattgtattattctgaaacttttatatgatggcatttgtgtcattgactcacacatgtttagtttaattaagagtaatgtcaaaataagctctttattttatctttaatcttttttaggtatatgtaatatctaagtgatgaacctgaaaatagaaatttttcaataaaactaggacattataccaaatagctataacttatgtagccccaacacctgggagttagttttgattcatctgcttcctttatttcccttatctggtcacaggttatatttacccagatatactttaaggttaactagctaatttatcattgcaaatgaatatgaatgaccccaaaatatcactggaatcaaggatcaaatattgttttttactttctctttagctgttgacccttagtcaaagatggcaagtcctctttctccagaatacatataatgtggtttggagataaaattaagtgaaggtttggtatctacatcaagttaaatagattttaaaatgctcaagaagtatacatatatatatataaaatatacacatattacattaaatgcaactgaagttttccttcaataatttatatcttagggtatttgaaggccatcttcataagtaaatatgtctgcttcaccgtaaaacaaaatctagtcatttgagctttattcagataaatgacagtttattaatttataaatatacaaattggggatatgaatatttactattttatggaataaaaatgagcatacttttccttgatcagaaagtcttttttctgttcttatctattcctttataaaaaaaagcagtagactgtaaaatattctattttttatttctattatgctaaaagttttagttacatcctgtttttcagtacttagaatccatgatttgaatgattcctatcatgtagttccaaatgcattttgtgtgcatctgctaagtccaaagagctctcctttatacttgtatacttactattaaaacacttttatttattttgctggtaatggtgacacattcaaatttgtttgccgatagacttgcatatgtattccctttgtacaggatattatttttttaaatgaaataggtttgaatcctatttctacctaccggtactttgaattagacaaattagtaacttctctaactttaatttcttcatctgcaaaatgaacaaaataataactaccctgctctgaaatttcaattggattatatatggcctagtaatgactcctgcttttactcttaaatactagctttctcctgctcctttctcttcaccttcataactcatgaaactaagtaacataaccagggaggcttttaagtgccaaatccaaagaaaagaaccaagttaactggattcaaagctacttaaactgataattttgaaaccatttgagcatctgccattttctcaaactcagcctcaccatctaccctcccaaaggctcctcttccccatttacctgtctctttgtgagtaataccacctcctccaaagcatgcagcccccacacctaggagttagttttaattcatctacttcctttatttcccttatctggtcacagggacataggctaatagagttgaaaaggggcttcgagattcagctttcttcctttgcagagtggttctctcacttctgcagggtaacttcctaatatcaaggaggtccccctactaacaagacactgacactcttagaaagccctatttgtattcatccaaatctgtttctctggaatcccacaatcaccggttagagttccttcctctgcaaccacacgaagtcctaattctccagcctaatgacagcccttcacatagacaaggcagcatcatgtggctcctaagttacttttttttgttgttccttggaataaatgtccacagttcctttaactgttttccatataacatggtttctaaacctgtcatcattcctagtgctcatactccttcagacatttttctgggacactcttaaatgcggtgtccagagttgggcaaaatactacagataagatcataccagtgcagagcgtaggagacatgagatatgacacccagcttatgaccgcatttgcctctggcatccacagacttatctggaccttgtcatagaatcaaaatcctcagttcctttttatttgaaaatctcttaaactagaacattcttaatttatttttgtgcagtcagtttctgagcctaaattcaaatacttaggtttatttctttaactattgatcttttcaagtctcatatttttgtatgtgtctgatgcatcttcctttctataatattcccttctcttctcaatgctatattctagccaggaatctttcattgcattggatctacatgattcttcggaatgcacatttatggctatgtttaaatattaatttatcctcattaattattcacattcactgcattgttgtgtggacacctggggacttgactctaattccttattgcttttctattgtttttctccttagaattactagaaatccctccactaattcttccatctatgtcataattgtttactataccatctttttgaattttaaattaatttcagttttaattctttttgattgagtcagaacacctctggcccagtaagtctaagtaacctgaatcttgtgtttggcccaacaacaaggtattgttacccttgatctagaaaaaagaaatcctgttctctaacaccatttactttcatctcccttcctccgtcctttccaaagttatagccttcgaatgcagagagaaatgaaaacaccacctgtgttcccagactttccatttcctatctagggcttcaacatcatgagagataaatcccaggttccttcagatgctgcaatgcagccccatatgattcagcagaagtgggtagcagttgggtttgattagtcttggcaggcactctgtcacatctcaagaaaaggctccaagaggaccacacacctcccaattagccatgtcagacttgcatctggctttctctgtgtccttcagtaggaagtcaacaccacccagagcgagtatcaggatttccagcatctgctatctcctgtgacaacttctttttgttctgtacatcagaaggtgcggcttcttttgaagatttttgactcaccttttatgggcagaattttgtggtgcaatgtcactccataatacagagattcttaattaaatcattcagcatgagtgtatttaaagacaaatcaaatgtagatcttgtcctcacgaaatatacagtctagaagaaagatgagagatatatgttgataatatgataattaatattttattgcactttataaagcactctcagatatattatctcattaaatctacacagcaaccccataaggtagtctttattattctcattttttcccatcagaaaactgaagtttcaagactttaattggcaattcagggatcatccagccaagaaacagcagaaatgagacacgaacttttatcttctgagtccactggtcttcctttgccactgcaccctccttctagaataaaaggattggtgaaatatcgtggaagttttagtactgacaatacagcatcctctatttcatttaattttcacaaccctataataggttaacattatcctcattttattaatctggaaattgagagcattaagatgattgagaccttgtccaaggtacaaagaagtaagtggctggagtagctgttttcagggctttcttccctctgcatcatgctgtctctttccactaaacttaatttccattcttttcctcagaagccttctgtcttgcataaatgaaatggaaagaatacttctagaaaatgattcaagaacggctttgtggaagagagagagaagagcagagctgtgactctcaaattttaaaggcttaaatgtcattaaagtgaaggtttcaagcctctgccccccagagattgtccctagattcagtaattcttgagtgggacttaggagtctgcattttctaaacttctcaagcgtgtttgtaacaggtagattagagagactttactttgaaaaaaaaaaagaactaaatgaaagtttctcttcttactctttctaccctttccaccttgtggtaaggtaatgttctttcactcaccaacttgattactgattttaagtccactttgcttccttagaatttatggatttcattttaactcttccccaccccttcttccagccccccagacacctacttttggaagcctccatcatttccactaaataataaagtaaaccagtgttcttcataaggtagctgttccctttagccagggggatcagccattccttatttaacacctaagtgggacttacctcaagtaaaaagatgaataacacaaaccctacagactaggagaaaggtaaccactgattgcatttctggtagaattatggagagtgaataatataatgggcattctttgaagcttgcccttgaaactcttcctgccaactctctgtttcccatattgtacactgctgggcttccattgcataaatcttcctaggatatgaccttcaagatggccttatattttttctcctaatccagaaggtattttgagtgactagtaatgtgtagcattgaggtgtcaacccagctaccttcagcagatgttataacatacagaatcagctgagaaggacccgaagtttccacttcttttgccaggtctactttcgggtttgttctttctcagccaccacagctattccagcagctcatttcttcacttcacatctgttctcctagtcaatgccctaactcctcacttgtggttccctgattcttccggtcctgtgggcctgttgcttctggtctaatccaaaacaaacaacaggaataaatcacttcaacagtacaacttgtagggatcctgtaaactgttatactacatgtaaaatactctgcttgatgtttgcagccctttataacctggcactgctctacaaatttaaaaatgtatttcttattcaaccttagcttcagtttatcttttcactgactcatggcttactagattcaaattccttcaaattccccttttttgagtcgtcagttgcttcttctcttaactgtcactttgtgtgtgtttctaggtgagcattattacattgtgttgtaataatttgtcttgctttgtttagtcttggtctgtatgatcagctagacagagttcctcagagttgtcaaccatatgttaaatatctttgtgtcccagacataaatgcccacacctaacaagggtataatcagtgcttattgacagatgaatgaaaggagtgatgggaacgttgttatgcctgattcactgataatgtaaacacctcttttcatgctggatgccaaacaggcctttgatcaacactcttttggcttcgactaactttctctcttttaaaaaatcttcacatttttttaaggtactgctagttgaacagctcttccgcaaggagttccaaccatttttgaagaaatggtatcgtgttattggattaagtggtgatacccaactgaaaatatcatttccagaagttgtcaagtcctgtgatattattatcagtacagctcaaatccttgaaaactccctcttaaacttggaaaatggagaagatgctggtgttcaattgtcaggtcagtttaagtgtttatttttttaaaaaaattaatagtaatattgatgataacaaaggctgtatttgttaattttttaaatgttcgtggcttaaattgtcttttattgcttatggttttattaaaatccttctacttcctggaggaaacaaaaaaagtaagtttttaattagaccactttattctgcataagaataccattattaaggcaagcactcgctggactttccatttattctgacaagttcctttatttgctattgaatgttgttagatctgcttccctttcccatagaataaaaatacaaatcttgtcttttatggggtataggatgagaaggtaggagaaagaatggaaaaaaataaaaataatttataataactaaatgcaatgttaaaacaaggcaaaattttccctctttaaatagtgtgtttttcacttgatgtaataaaaataaaattcaccaacctaaaattaaatattttattaaagaacaaaagtaaaatcctgagatttgttttgtaataagccagggtgtgaattccacatctaacgctttttagctctgtaaactttctgaatcttatctattaaagaaggagaatgtctaacagggagtttcatgattagtatatacgaacgtctgtgtgtacaggtgctagtatagttagatttccctcctaaaaactgaaagtggactgctcttgtgtcgtgcccaagagaaggcatccccaaggcagctcaattactggtctaaagctattttctcactgtagtgtgctgatatggagaaatgaactctttgaattttcttttaagacttttccctcattatcattgatgaatgtcatcacaccaacaaagaagcagtgtataataacatcatgaggcattatttgatgcagaagttgaaaaacaatagactcaagaaagaaaacaaaccagtgattccccttcctcagatactgggactaacagcttcacctggtgttggaggggccacgaagcaagccaaagctgaagaacacattttaaaagtaagtcataattttatgtataaccaatgaaagcaaagctaaaacaaatgccaggacttcttggtcttattaaataagattgagttcaagtgctgtgatcatcagttaagtgaaatctgtttgctttagtacaataaatatactcagtcttctacagaatactaatatgccttaataaatatcaataacacaaaatcatatcataaaaatgatggagagtgatataattaatgggaagttgagataaatagaatgccttcttttgaacatctactcttatggttaaatttaatgatctacctgatatagaaatatgcataaactatgaaagtttctagttgtcacacataataaagtcttactccatgagtttcaaaactgcgtattttctgtccttcatatgttattctaggaatctaggtaattcaaattaaagggaaatagggatatgaagatctttaaagatgagcttgtcagctgccccctaatggtaacaccacatacagtgctactgttaatcttgagtgttaagtaaatcagagtagcccaagaagtattttggtaccaaggcaattgcatttgaattttaagtcccttactttaagttgaattcaatacaaattatgtagagtagacacaacattcagagatgcaattataggctccaaaatataatttattactgggattaaggactaattccaatctaataatcaaggaacatgactaaggatgctcccagctttatgtgtagatgcctgctttatgtgtagaagaaggtggtagtatgatccacggatgtcaagttgacctttgatatgcctcatgtaacatggttatctcaaaatcattctctgtcaatgatggtaaattgttatactctttccatgtcagttttccacttggaaaatggaatttaaggaatgaatatcaatacacctttttctctgttattacaccaacttgatttagaagctcctttttcctatattaggacaaatgttgctgattgtgggttaatgagatccataataaaacaatgttaaaatgctatatttagcaagcaatgctacatattactttcatgaaattttataccacatatttatgctacaatttaatgctacatattttttacatgaaaaagagttctacgttgaataaagtgaaagggaaaaataattgcataaatattgaaaaatgtctcttttccagctatgtgccaatcttgatgcatttactattaaaactgttaaagaaaaccttgatcaactgaaaaaccaaatacaggagccatgcaagaagtttgccattgcagatgcaaccagagaagtatgggcttaatgttttatatctattgattgacaatactacattcagtagaaagatggcaaaggctattttatactgaccatctgaaaaatattatttaaaaattttagcaaacaagtttcaggtttttttttaattgatgttaggcagtaaagcactacagtgaattctcgcctctagacctcttttagttttctaatttacatgaggaatagcttattcattgtttgtacatatgagttaaaagggacagtgaggggccatgggcagagggatgtgtaactaagctggaggggttatgacagttcgttttagcctttttaattttcttcgtgaattccaggattgattttctgacattccacagagccattttgttgctatcaaattacatttggcaatgataatgggaatcctatctaatttgcatacttgcatatttactttcttaggaaaacagtgcaatggtaaagaagtgaaatatttactttaatgatgcttttctgatgaatttttcaggaattataagaataaactgagagtcaatttggacattatggccagtaaattatgtcaatactttattttgtaaaatttagaaagcattacaaattctggccatatgtacttgataataccattgaaaataagaatgttgggatcaaatgatatgaaatatgcatagtagataaaacaatgcttgaaatatagtaggcactaaaccaatgttagtaactagcctccgtatatctctttttttaaattctgaaaaagactgctatctactccagctgcactagcacagttgaatatattttattcactttcctttttttgttacttatttggttttacttgataattttaaatgcatgtttctactcataaagatttttaaaactaaatttctcaggtacattttttaaaagaatttgccattttttaaacttactttaatttttttatataccttgaagcataacatacatacagaaaagtacaaattcatatgtgtactgattgctgaattttcctaaactgaatacacatatgtaaacaaaatccaaatcaagagacagcacactgccagtatcctaaaagctcctggctcccccttccagtcaccatgccccaggaatcactactatcttgacttctaaaacaaaaaatgagttttcactgattctgaactttatataaatggaatcatatagcatgcactcctttatttctggctttattcagactctacttcaactttttattttggtcgaaataatactgaaaccagttttacaaaggaggaatgtttcttaaattacactgtaagctttctaaataattgtggcattaatgcaatatgtgttttctaagttacctttaacatacaaggtttatataattcattaactttacttttgttaaccttttaagttggagattccagcagaggtaacagggtgttggtgtgggtgtggcttgatggcaggcttaaaagagaagctgacaaaaataaatgtacagacaagtgatgggacactaacctgttactttgtctaaacaaagatgaatcagatttattataacctagaaattattctaacaaagataatgttattcctttaacaataagaatacataagaataagtgtgtactttatttttaggatccatttaaagagaaacttctagaaataatgacaaggattcaaacttattgtcaaatgagtccaatgtcagattttggaactcaaccctatgaacaatgggccattcaaatggaaaaaaaaggtaatttaggtttggacctaacacttagtttatcccatgtttataccaagattttagatgactatatcacagattccccaaaaaatggaaagcaaaagtagttccaaatagaatttctgtgttctctgtaggacaagcataataaacatgttctaattccttttctaaggagagagattccaatgctatccaattttctctttgtctccagagtcttgagataaactttaatagctctgtctccaaaggcaaactcaactaatatattttcatgccatttctctgtgcatcagctacatgtttttgctgaggcagaactcaagcagcaagtctaggagttggaaaacttgccctgtttcagagctcaggacacattttgagccttaacttgcccattttaaaaccaaggtgaagccagttttgcagttagtaacatatattcaagtccatggcacaattttagggggtttatatgcttattatttcacttacataatatattttttcacagctgcaaaagaaggaaatcgcaaagaacgtgtttgtgcagaacatttgaggaagtacaatgaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttggtaagattcaaagtaacatatactggtgtcattcatttaccttttcaacatgaacttactgcttctcataagctctccaaaaagagaaatatcttttgaaagaagaaatcccactcagattggaactatttctaatgatatgaccagattagctaccagttgtatgatcacagtcagtcatttggtcattcagttttattgtcaagacaattgtaatgaatttatttattaaaccatcctaattattagatatagcgcaagattgtagatgagccacaattcatgaaaccttctagtgtaattcgttgtatgttttactaatttatacttccaaatagtttttaaattaattaatttttaatcaataaaactgtatatttttggtgtacaaaatgtttgatatatctgtacattgtggagtggctaaatcaagcttgttaacatatcaactctaattatttcttaaagaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacaggtataaatatccttttggacgacttcttttcttttctttctcttttcttcttctcttctcttcttctcttcttcttttctttcagacgaggcctcgctctgtcactcaggctgaagagcagtggcatgatcaagattcactgcacccttgacctcctaggctcaagcaatcctccagccccagcctcctgagcagctgggactctaggtgtgcaccaccacacccaactaatttttttatcttttttgtagaaaaggtgtcccactatgttgtcctggcttgtcttaaactcctgggctcaagccatccttctgcctctgccttccaaagtattgagattataggcacgagccactgcgctcagcctggacttcttttcttatgctgttattactcttctcaaattgtttttagtctttttctaccaatcctctcaccacccatgaatacatttacaggataaaacacctgttcttgaaataaaatctaaggctaatagcaatcaaatctgtggtttttgagaggttaagtgttgttttaggagtcatttttcccacataataattggaagaactttctaagtttcccttgcaaaaataagtgacatgtaagcttaacaacaaagttcagaaatgattgccatgtagtatcaagtggaaaattcaaactcccacaaaaatgtgttcgttataactagaactatgcaaaatatatttacatagaatgagacctctagggaaacttgaaaaaactaaagaatttgatatattagagatttgggattctggtaagtaggttttctgttccattttaaggtgttttgttttactttgtttacatgttgcttactgtgggcatagtaaaaaaacacttgggtaagaaaaaaaggcactagtagacattacaagcatgcatgaagcaatgatgccttgttagagtaaaatattcttctgagggctaaaatcacattgaccctgagtctttctggacaaatggatacttcctttctattatagttatcactattcaagccaatggagcaaattaattctgtttcagtgtttgctggaaaaaaattcttgaaatggttcactataactctttatataccctctacatgcactcctgtgagcattgccaataaaattaatgccagagaaagtactgttttccttgggtaacaagtttgcttccttctttttgatgagatgtagtttgctttatatgtttttctttttgccttagcttccagaaagctagcaggaaaaactcaagttcagtctcacagcctctgtgttgattcttatgagttctgtttttaaatttctattatattaactttagcgttgtgacttttattgtaagagggctaagcttctctgtaaaaagaggtagagtgcaaatgaaaagcctatgttcaagctactttctgctccttattcatttcaaaccaaggggctgtgtgccaaaatacagaacatattcccttccttccccactccttgtatcctcaccttgtaaactggggtggacaaactccttagcaatggtgttaggcatctatgtgatgtgacttgaagatctgacactgtctggaatttttgttattaaagttactccttcaaattaagacacaggcatcttctgctaaaatccaagaacatgtaagtttttatcgggagatgtgagggccggagggagaaattttcagagaagactcaagagaggcctttctacagggttctccgacagttaggagtaggagtcaaataaggactcacactgcctctgagaagaaagggaaggggatggaagaacatacttttggtctggcagctttatttttgtttaatgtatgggggcaatagacagtgtggatgtgcaccaaatgccaactggaccaacagctttgatgagagtggttatgaaaataaaaccacattgtccatatcaagctaggtttgtgctcactcatttctcccatgtaaggatgagaggtcatattttaatttatttatattgactttcaaaattcagagttttttcatttgaaaatagttttatttacagttgcataacctcttctttcatttttatttatattgtccctttgaattgataatatatttacctcattcaaaattaatgaggcacaaggcataccactgacctctagtcttccagctcccctccctagatgcagctgatgttttcagtttctcttgtgtccttttgggaatattccatagcttctaactcaaatgattcttaaagtgaccaaatgcatatacagtgtgcattttctctgtgactgaaaacaatatatcaaagcatctctgcatcttatcctctaaatagcaaagattctgaagttaatgtagtgggggaaaaaagcccattttagcttctgattgtcatatcctcttctcaaccgacagctcctcctttcttcctctggagtcacccatcaatttatttccttggcataaaatgtggtcttcccacctccagactggatgatggaaatatttttgttattgtgtggtaatcccagattggaaatcaaatgttgtctcaccattatgtctttgttcaatgccaaaaacagatggcaacaaatccatgtatgatctaatcctttgatttacactgaccagttgcaatttattatttcttcctctaattagaagattttaatgtgtttagcatcacaaaatttaatttattaaaatgtttctacagaatgaacaaaaagaagtcattagtaaatttcgcactggaaaaataaatctgcttatcgctaccacagtggcagaagaaggtctggatattaaagaatgtaacattgttatccgttatggtctcgtcaccaatgaaatagccatggtccaggtacattcaaatacctctacttactatgttaatttttatttgttgtgttcctattttaattgctttcttttagttaaacagcaaaaacatctttttagtttttaaaacaaagcctttttcctgttaaaagagcatttttaaaatgctgtgcactgttgcctttcatattgctgtcaattcatataataaccagatacctgttgtatgtttaaacaacaactcctctttagctatttctgtaccctaaggctttccaggaccatgacactagccccatttgtaagagtcaaaagcagcctcctctttcctaggatgtctgtgagcccccagctggaccaggaatctaaagtacactgtcccacaaatgtctgatgaagtaacatgcccaaacctgagggagccctgctgtttctgcattttcttcccttatcctttccaattttctgtgtgtttagaagttttcatgataaaatgttggttgggggcagacttataaatgtaaaaataaataaaagagtgaaatgttctgttttcattcagatttcactttatctcttacccacctatgttaatggcattattgtaacctgttgagtaggcataggtgttatgctgattaatagatacactattcttcctataaaattaactaaagggatttttaaataacttaaattgcatccttgaataaaaggcaagtcatagaagactaagataaccagactgccatcaaattagcaaataattttatttcaggagtctatttgtaagtcagtgataagcttagtaatcaaacttagaatacatttttccttgaggatacttttatagttgataggctcctgggccaggccacagaagtttctattaatcaaaatatacctgaatatcactttcataaagaggcttgtagaaagttatcaccatgttaaatatgttcccatgggaaaatgtgctaagaatgtaatgtaggatgccagaaactgacctgccttctcaccagccctagtgtcaaggtcagagatgacccttctctccattcattcctcccagcttcctaccaagaaagacgactcctcagtccaagtcattgatcgtggttctcatagtttgagagagaagttctgagagcaaaaagcctggacaggatgatattatgtcattgaggcctaaccatcatcactctagaacactttttctgtattaaagcatattataagtttcaaagagctgacttacaagtaatggattaataactaactttactaactttacccatttgaagactggcatgctgaacaacccaatatcatttcattctgagcaagaatatcattcccagaaaaaaccaggaggatttgtatcacaacgtaccctttcatttacttgatttatttgtgttttcaggcccgtggtcgagccagagctgatgagagcacctacgtcctggttgctcacagtggttcaggagttatcgaacatgagacagttaatgatttccgagagaagatgatgtataaagctatacattgtgttcaaaatatgaaaccagaggagtatgctcataaggtattgttggtcacctctgaatttgattttcatttaaaaatggcggcatgtggttgccattgatatctctgtgactcattctccagacaaaaattgtgctgaattataagtttctgtttagtgaactgtgcttaatccagattcacatgcagagaatcaagatccatggtgggcaggaaattatcagggagccattcctcattccagtaaaaccaaccataaatttggataatataaatttagcctgccttgttccagaaagactgctgcaactcactgattcaatccaacatggtaattttggtttgatccaatttgtttgtaaaggcacagttttttaatttcagaaatgtagatgaaacgcctactgtgtgccaggtaaccttataggaacagccaacagaggaaacaaaacacataagactcctgctgcacgaagtttatatttctagtgtgtgcctggtgaagtttggaggacagaaacagtaaacaaacattaaaagacaagaaatataatatatcaagataagtgaagagaattagaataggatgatatgaaacaactgactagatggatactttcttgtttttagtagtttttgaaattcttttttttcttattatactttaagttctagcgtacatgtgcacaacgtgcaggtctgttacatatgtatacatgtgccatgttggtgtgctgtacccatttacttgtcatttacattaggtatttctcttaatgctatccctctcccctccccccaccccacgacaggccctggtgtgtaatgttcccagccctgtgtccaagtgttctcattgttcaatttccacctatgagtgagaacatgcggggtttggttttctgtccttgtgatagtttgctcagaatgatggttttcagtttcatccatgtccctacaaaggacatgaactcatccttttttatggctgcatagactagatggatactttcaactgggtggttgaggaaggtctcttagaaggagtgacagggaagctgcatctgaatgataagaaaatacagaatatgtcatctagctacatctcaatttactaacacacagttatttgagttgtgcaaaggaatgcagtgaagaaagaatgtatttctatagtatttcaggaaatcccccataccactgaattaaaaactgtaactactataatagcaaacaatccatttagaagaaaattcacacccttgcatatgggtgtgaatgttagtttcttaacattggaattggctctaccttaaatagagtttcactaaagggatgctttcaaattggtgtgcattgctattaccaactccaaggtttcctgaccaggtttctgcacaaaactttattttaaaacccctttaaatacaaggcaaagcaaatattttagagtatcaaatcaaacttttgtcaccaaattatgccactggaaagttactgcctcccagttaatgtcctaagagaattaatcaagtagatgtttacaaagactgatgttttaagagtagttttttgggggtcaacactaggacaatctctggagtttaagggtcaaattcttccagacattctttagaataagactctgtccctttgggtgccccagaatggactatatttttaggtctcctttggccaagaaacctgccctcctaaaactacttcaggtagatgaacttagagtaggagtggcaatggggaaggagggctacatgccaccagtaatctgttcaaagcctgtagaaaacattgctactgggaagatgtttactttcttctaatttgagcatctttctcaacacaatcttcagacaaccactaatgaatgatgagaattgacagataagatgaaatctttcttccatgtcataaacacagtttaaacataacagagcttcttaaaattaaatcagacttctaggagcaagttgatcatcataaatgtttttctctagcattgattcaatttttttgctacattattacactttatttggacttaggctgattgtagctgtgcctcctgtatatttattatgagttcttttttttgtttgtttacccctctagttacttcaaattcctttactattttagagttagtcttttagcttctgatgtcctgattacaaatccatacacttaccttggctttattttttttctgactcattcatacacatttcttaattcccacatgtatgtgatcctataattaccttatcagctcattgaactgtaaatgtcttataaaagtaacctattaaaacttttagggaaaaatatttcagtgtattattttgctataatttacataggaaattaatcttggtacttgtctgattaatctattatatagcctccaggcttgcttgcaaggttttattctcttttctatgctatatatggctgccttgaagggagatctaaaatgtagagttagaagccaaagagatgggctttgttcaactgcactgatgacttacttttactgaaaatttctccctcggtcttaaagaaaacaattaattcttccataatttaatataaatatgtcatctatgagaggtcagtttggcggtaaattattcaagaaccagatttttctaccgtgaagttgtgttggtataaacactgttcagtccctatgtcaactttcatatgttacataaactagattatttccattataaaataagtgctgctgctagtcacagagcatagaataaagaggttgatgggcatattctctcacagaaggaatctaggcagtattggctccatgaagttatgaggaatccaggccttttctctcctttttatgctgttatagcattgggctttcattctcatggtcgaagatggcttttagaatttcagctgccacatcctcattctaggcaggaagtaagaggagaatggaaagggcgaaaggtacacatgccagctatctaatttttaagaagattttctgaaaattccactcaacagctttttacatgtcattgactgctggtttggaaatgtagtcatttagctagcatatttccacccagaataaaattatggttctgttaggaaaaatggacactgaatgaaaaactagtggtttctgctacaaatgtgtaaagagtagactgattaagatggtgaagggtctgaaaattataaagcatgaaaaaagattaaaggcatttgggtgcttagcatagattagagaagacttggtaatgtaataacagtgtttagtaaagagattccatgtggaaggaagtttagacctattttatggaacagaattaaaacaaatgggtaggctggcgcagtggctcacacctgcaatcccagcactttgagaggctgaggcaggtggatcactgagatcaagaatttgagatcagcctgggcaacatggcaaaactccatctctactaaaaatatgaaaattagctgagcatggtggcaggtgcctggaatcccagctacttgggagtctaaggcagaagaatcccttgaacccaggagatggaggttgcagtgacccgagatagtaccaacacactccagcctgggcagcagagcaagactccatctcaataaataaataaataaataaataacaaatgggtaggctagacagagaatttttgttgaatattgctaaggactttctcacaatagaacttttctgaaatggaatgggctactttttgaatacctgaagactgcacattttcatggttacaggagatgattatataccaaattctttggggggtcattacctcattatttttatttcctagaccataagctaagggtatattttcacaactaagacctccaaatttcaggagacttacttctatgaaactgaaaaaagaaaacaaacctgaaaccactaactattcctttctctgtccagattttggaattacagatgcaaagtataatggaaaagaaaatgaaaaccaagagaaatattgccaagcattacaagaataacccatcactaataactttcctttgcaaaaactgcagtgtgctagcctgttctggggaagatatccatgtaattgagaaaatgcatcacgtcaatatgaccccagaattcaagtgagtccagaaagacccatttttacatttttccacaaggggtgaagcaggttgcattgtaaatgttctttcaaaatagtgcaaatgagattcatttattttacaacttcctctcctttagcctctcaattatattcctattttaaatgtctatgttttaaataaactgcactggggcaggaaaagagaagtgtaagaaccaaagccattttgatccaaggcacgtatttttgtatgctcatcatttgttctcaaatttttgaacaactaaagtcaagttaatataaccaacagacttgaaagaagttatgaaaggcaacatgccccttgagaggtatgtgtagaagagaagcattgccttagttgcaataacaagcctgggaagcaggagggaaggaaggaatgccgtgtagaataccgcaggcatgcggatgagctccaggtccaataacccatgaactacacactggtattttgtcagttctccatgatgattctttccctttgatacttatagggaactttacattgtaagagaaaacaaagcactgcaaaagaagtgtgccgactatcaaataaatggtgaaatcatctgcaaatgtggccaggtgagtaaattgctgtgggccagattttactttagccaacaggaatgtttaattgcatgtacatttaattttcctcttgctctctcttttgttttccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgattaatgtattcattatgctacagaactgacataagaatcaataaaatgattgttttactctgcattgaa

SEQ ID NO: 2 - DNA, 51611 bp, Homo sapiens
  source 1..51611: mol_type = genomic DNA
caaactctgtaagaactgcctgacagaaagctggactcaaagctcctacccgagtgtgcagcaggatcgccccggtccgggaccccaggcgcacaccgcagagtccaaagtgccgcgcctgccggccgcacctgcctgccgcggccccgcgcgccgccccgctgcccacctgcccgcctgcccacctgcccaggtgcgagtgcagccccgcgcgccggcctgagagccctgtggacaacctcgtcattgtcaggcacagagcggtagaccctgcttctctaagtgggcagcggacagcggcacgcacatttcacctgtcccgcagacaacagcaccatctgcttgggagaaccctctcccttctctgagaaagaaagatgtcgaatgggtattccacagacgagaatttccgctatctcatctcgtgcttcagggccagggtgaaaatgtacatccaggtggagcctgtgctggactacctgacctttctgcctgcagaggtgaaggagcagattcagaggacagtcgccacctccgggaacatgcaggcagttgaactgctgctgagcaccttggagaagggagtctggcaccttggttggactcgggaattcgtggaggccctccggagaaccggcagccctctggccgcccgctacatgaaccctgagctcacggacttgccctctccatcgtttgagaacgctcatgatgaatatctccaactgctgaacctccttcagcccactctggtggacaagcttctagttagagacgtcttggataagtgcatggaggaggaactgttgacaattgaagacagaaaccgggtaggtgtctgttcagatggagctagccttttaggcagattttgcaaagcaggcttccttgtgcgtttgcccctttccaaatgtgcttgacctctttgggcaaatctagtttcttcctttgagaagacaacatctaggcactttaagacaattttaaaaatttaaacacaaaagaactacttttctaatgttttcactgaagtaaaaaggagttaactctagaatcgtaatgtgtattaacaggtaacttttattgaacacttactatgtgcagttggtatcattttcaaaatgtattactacagttcatgttcagagtaacccactgaggctcctaatgttattaactccattttacagatgagaaacctgaggcaggttaaataatttgtccaaggtctcccaggaaaaacagtggcagagctaagaatttagcttgtgatagtgttgaatgtttttcttatttaatgtagttccatggagcaggggttagtaaactacggcccatgggctaaatcaagcctgctactgtttttttcaggcccacaggctaagaatggtcttcagatttttcaatttttaattttcagtgttcataaagttttattggaacacagtcatgctcgtttgtttacatattgcttgtggctgcttttctactccaaaggcaattggatggagactgtgtgcccttaaagccaaaaatatttattatctggcattttagagaaaaagttttccaatctttgccctacattacactttgtgccatttagtcttagcccaaatgggtaaattgcaatcacacacacacacacacacacacacacacacacaccctttttctttaatcaaagtttcaacattctttgagaatcaaaatttagaaacaattcattgaaagaatgtgttgctagtttcagtttttcatgttagtgaagaaactcatttttcttaaggtagcattttgacagggcgaattttactgatgactttacattgaatgtgatatgaattattttttgataagtgaagatgtactgtgatgtgtataaatgctgacctgtttgaatgtgacttattcaggctcgtaagtcaaaagtcttgttttggaagacatgtagacaaataaagccatgactaaaagtcaaaggctttgtctacaccactctatgattttcacatggtttttttctctctctctcttttgggtcatccattgtttgtatattcatacaaacttctgggcatttgattgatttgctatagcctatgatgcaatttagtctttagtgaataaagttagtgatcttttgtagacttgacagacacagggcctgtccaaagacccaaacatgcatctaattttgatggccagttttcccaacaagtaaattactttgcactgttgtctgtaatgtttatgtcgttttgtgtattgagaaccagctagaacgcattatagtagtccagctggatatgacaaatgtgtacatcactgtggcagaatcttaatttggaagaaagtatgcagccttcaagccagctggagatggaagatggcatttccagccacagtagctgttagaagtccagaagcaacagagtccagtaacaccacaaagctgtttatgtttggtgggtatgatgtcccgattgccagcgaaacagcacttctccctatttactctaaatacttctgcctatcagcatcacttttgtcttgcctggattgaatttccgccagctagattttatccaagttcttgattctggcaagcttggaggcagctagcagtaatgcaagggatttgacacaaagaagatatagagtaacgggacttttgtgtactggtagcagttaaggccaaaacaacccaaattctccctctgattcacatttcaaaggaggccccaaaagactgagcagctataaatttgaggctctctaggaagcaatagtttctctgtgagagtgtggaataatttttgtgtgcatcatttacatgtattatcagttccaaaactaagatatggtggacaatgagacggaagcattaagtttggtagaataatctatattgaggtggtggtcattgacaaatgtttatcttacagcagcaaagtgtgataagactgtagaaggggaacacatggatttgaggtccatcttggcttcttactatatgcgttactttgggccaaattactgaagttccctgatgctacatttacctgtgaaaatggacatcataaaatttctcacagtattttagtcaacatcaaatcagaaatagtgtttatgaaaaagcttataaatcagagctatttgatatgataatggtgttgacttttgtaaccattcatctggttacatttctgtgtaaatttaagtttgagagattgaggtaaattccccatgtcgcaattgagatatacttcttaaagtccctagattatactgatttgaaaaagaaattacctctttcatcccagaacctaactaggattaggtagagggttggtgtttagtaagcattgattacttgattgatagactaattgaatttatttctgaaagtgtctggaataagcgttgaaaacacatgacgtattgaattgtgttgccaggcgttagagaatggatgaatgatgaaaataagatttccaaatttgcttttttgaaggcttaaaatagcatttaactaagtaatcaagacttctataaaatggtcttgtattttcattctttttctgtttccccatagacattttctacttgatctattattttaatttaggattcccaccccatttcctagcccctgcccgtaattacagcaggccttctgaaaatgactttatacataggtttgtgtccagcaagtacccaaaaacttattcattcattcactcattcattcagtttgaatttattgagtggcatctttgtgccggatgccatgctagtggtgaaaagataggagaaaaacacagagactgttttgtcctttcgtgaagttgtgttgtagtgttaattagtgttgataaaagaatgtctactgtaggccagtcatggtggcttacgcctgtaatcccagcactttgggaggccgaggagggtggctctcgaggtcaggagatggagaccatcctggctaacacggtaaaactccgtctctactaaaaatacaaaaaaattagctgggtgtggtggtgggagcctgtagtcccagttactcgggaggctgaggcaggagaatggcatgaacctgggaggcggagcttgcagtgagctgagatggcgccactgcactccaccctggtgacagagcgagactccatctaataagaaagagagaaagaaaaagaaagaaagaaagaaagaaagaaagaaagaaagaaagaaagaaagaaagaaagaaagaaagaaagaaaagaaagaaagaaagaaaggagggagggagggagggagggagggagggaaggaaggaagaaaggaaggaaggaaagaagaatgtctactgtatacaagggactattctaaatactagagtttcagcaataaatatatgccattttatcaatcattataataaagcaatccttaatggtaagggggtacccctgtactgccttgctcctcttttaggatgctaagttacaatttggcattgttacatcttagttataactaagttacaacttggcacacccatcaatttaggcagtcatgaactctgggaacgaaagtgatcatataaacatttccatagtgtcttaataatcaagctgaacagctaaagagagatgcatgtttcatccctggccagttcagaacacaatgcatctgtagtcctggagactccacacaccttccatttacacagaagaaccagagatgccatgactagtccacctaagaacagtccagttctcccaacatgaagttgagcacccaagtcaatctccccaggaacattgtattgtctggcaaattaggaaagataagaatttcatctgcaaaaaaaaaagtatgaataagaaattagctatgtcaatgtgacaataaatattcattgggttaagacattaactttttgtgggaaactggttctttaaaattcttttttaacatacaggttttgcctcctgatttacttttatctgagagttgaactctgctgggttaaaaaaatcaccattatatttaaatagctatctaaggttattataaagggaagaaaacattttcattaaactgtacaattgtgttggtaagccagtgctttatggaaatgtcattggtttcttcactgatttctgttggaaagaaaaaacatatcaaagaatttagaatatgaaagcattgagttgtattattaccaaattaattttatttcgtatttcataaattagttaacaatgtattaatagactacatagtgaaaacatccatccatttttaacttcacagagaaaacttcgagtgggggaggggcatgggtgagaattgttttatgacttgttgcagtctcaaatgtaaatattcacctcccacgaatttctgtaattgtgtttacatattgggatcaatatctttacaatgttttaggagaacatattaaatttttaaaattatataattgcttcaaagggagctgatgtactttccagcaccaaaactgagaagatttcttaaaaattcagcatcttgatgaggctgctaaaattgcacagatatatgtagttccacattcaaattgtaagaatatgtttaggaaataatgacatcatgatccttatatgcttggactcaataacggcaaagaaagcttgaaaaatatgattccacagtaaaagttgacttaatagaatagtggttgaagaaaattttgtggatagaatatgcttagggatttttaataacagcatttgttgcctcactggttctttttaaggaaatccttcctgattgcttcagtcaaatatatttaaagctgattaaaggatggggtaataaaatatgagaatggctgagttaaaagaagacactgaaaccagtcttatttacatctatagcaattacttgttaaattagcagatgataataaagatgttacaacatcagggaagccaaagaatttccattaggagactaagagagctagaaaaaaatggtaaaaaatgagcaaaaatctgcttagaaaatgttgtgggtgacatggactgaattcacttgaaatttactgagttcaaatagttatttttaaccacttcttgccagaaaatgaatttaaaattggagaaaattataagctataattatagttcaagaaggcaggattcagcctctaaattcaactgcctgggatcaaattctacttctgccacttactcactttgtgacctcagagaggtacctaaacatctctgtgcctcggatttctgatctgtaaatgagttaatattggtacctatttctgggctgctgggttaatatattcaagttccctagaacagcatgcttaactactagatgctaagacggaaatatcccttgggtagagaccatgctgtatgtgcttgtgcaggtgaggtgtttgtaacaataactcttcccaatcatccataatagttctaacactgaattagtgccagacattgttccaaaaatgttaacaaatttctgacataaaaggttatggaatatatttatagaactagaaaatacataaatttgtaggaaagaactaaacaatccaaatgatacatgtggaagattggcaaatgttgagaaggacctttgataatatcaaagtcaatatttcttatctgggaggctgatagaatcacatgagatgttttactttttctttaatatctgagttcctaacatagacaaaataaatcataatcttttaggggtgtttcctagacatcagtattttttaaggctttacggttgtttctaacatgcagctaagatatagaaactctgaaatttcttttcagctgccttgtaaccacatattgtaaccaaggtctagggagatgaagtaaattgcctaatgccacagaaagctaatgatgtcctgcctaacttctttcatgtctaaacatggcttgagttactctttcctccaaatccttattgctatattaggacctagggaaatgcactgtgacaatgattacacaaaagatccacatttaaaagaaatttactaatctaaaagagaaaataaaagaaggctaaagactctaatgaatggatataaaaataattctgccccatatttccaaaaaaaagatataacaataaatttcccataacattaaaaaaacacaaatttgagaaaaattgattatagtaataatcaactatgcaaatattacataggaaaaaatataaatctgcagtagtaattacttatttcataatgataatcatgaagaaaggagataagaaatgtttgatttcccatttacagactccatagattgaaaaaatatagccagccctagatatacagaaagaaattggtgaaaatagacataacatggctgcagactaaagatactttctcttctatcctttattcaggaatggctgaggtaaaatttattttctaggtgtcaggtttgaataattcaatagcttttaaaaaaaatggttcatatttaaaaggacacttggtccagagatttttgttgttgttgttgttgttgtcccggagatttttttccctgccgacttcagaattgctacaggtaacattgctattttcttcaatgtttcaatgtaagcctttatttatacttatgcttaattgctcagtttttctaaagggtatttcctgtttaaggttattcagaagatgtttgatcttacttaattctaattttctcttacaacagattgctgctgcagaaaacaatggaaatgaatcaggtgtaagagagctactaaaaaggattgtgcagaaagaaaactggttctctgcatttctgaatgttcttcgtcaaacaggaaacaatgaacttgtccaagagttaacaggctctgattgctcagaaagcaatgcaggtatttgtaattttactgaggaagattcttcaaattctgcctagtgattgagaattcaataaagcagaaagacacaatgctaaacaccaagatttttagaatcaaaattttaagagtggaacatatgtgatagatcatctattttatcaccttaacaaatgaggaaattgatgcctgggaaaatatcaactgatcaaagaccatttttcatccaaccaacactctttctactaccttattttaaaaacaatatgactaccagaaattttatcaagattatgtaacattaaatatacacaaatacattggaagaaagtattttagtggttataaaaaatctgctgcatattcttctgaacatttttatacattttaagcaatgtattaagcatttgataaaaggattgttttgtcaatatatctcaaccttacctaatgtgagattttaaacagcattcaataatagtaataacataacattttatagctccttctcctacattatttgtaaatctcacttaaactgctatcttatttttcttttcgtagtatgcatcctatgtttccaaaaatatcatatgaaagacaaattgaaagttaactattaattaattataatcattgcagttttgttctggtgaccctaactaatgaaaactttgctgtaaaaagtggggtgctgctgtaacaaacacctaaacgtgtggaagtggctttggaactgggtaattgatagagactgtaagagttttgggtgaatggtagaaatatgggtgttcagggtgattctggtgaggtcacagagagaaatgagaaaaatgtcattggaaactggaagaaaggtgatccttgttataaaatgggataaaacttgatctcacaagtaatggatttggatatttggctgagaaaatttcttagcaaaatgctgaaggagcagcttgatttcccctaactgcttatagtaaaatgcaaaagaagagaaattaattgaaggagttgttatgcaaaaaggaaccaaaatttaaatatctggaaaattctcagcctatccatatggcaaaaaatgagaaattgtgttataaagagaacaaccaattgataacaagatcatgggtgctactgatggacttaatcagccatttcagcagaaggtaggagtagagatatattataccagcaaatacacttccagtttgaactgaaggagacagagaaagcaggacagaatgagaaaaggatactggactttctggattctacagaaccagaccatagagctatttggctgtgaatgtgtactattcttcagggaaaggaaagaatgcccccaaaagtgatacagaagtcatcagggttgccacttccaccacagacccagaggccaaggcagtttcctttttggtttcaaagggcaaaactgtctctctggttttagtgagccctcaggatgaccctgcctagtgtctcagaagcagagctatcctcacagaaagctgtgtggatggagcccttagagagagcaggctgtcttgcagagccctagaacctgccattccgtggacctggaaggcagagcatcaaaccaaacagagctattctcaagccttataagccttaagatgtgacagaatttaccttaataagttttggatttgcttgggacccatcacccttgctttctttcctatatctcctttttggaataagacgtccatcctatgcctgtcccaccattgtattatggaaaagaaacatgtagctcaactggtttcacaagttcacagctggtgatgaatttgccttatcatgaatcacacctctagtctcatctacgtctgatttggacaatgtttaggtgagacttttgactttagacttagagttcatgcttcagtgagttaaggcacttgggctgatgagatgagatgaatgtattttgtatgtagtaaagatatgaattttgggaagccaatggtaaaatgctacagatggaattttgtcctcctaatactcatatgttggaacctaatatgactctatctggagacagtgatagtaggagatagttaagattaaatgagccaggccaagcgtggtggctcacgcctgtaatcccagcactttgggaggccaagacgggtggatcacctgaggtcaggagtttgagaccagcctggccaacatggcgaaaccgcatctctactaagaatacaaaaattagctggggtggtggcatgcagctataatcccagctacttggaaggctgaggcagaagaatcacttgaaccaggaggctgaggttgcagtgagccgagatcatgccactgcactctggcctgggtgtcagagcaagactccgtctcaaaaaaaaaaaaaaaagattaaatgaatcataagaatggggccttaatctgatagaattgtggccttttaagaagaggaagaaagagaaaaagagaaacatctgtctctttctctctcttaaccgtgtgaggacgcagggagaacgtggccatctgcaaatcaaggagagagtcctcatcagacattgaccctgctgtcaccttgatcttggacttccagtgcccagaactgtgagaaaagaagtgtctgttgtttcagccactcagttgatggtgttttattatggcagcataaacagactactgtaggagcttagactctgggtcctgatacatatcggatcctgatgcttatcaaatccaggctccttccttgaatgtgtgacctttggccaggttctcgacttctctatgcctcagtttactcctcagtaaaatggagataataatattacttatcacctcagtaaaatggagacaataatattacttaccatctaggtttattttgagacttaaataacttaatgtatgtaaagtatttatattgtgagtgcaaaataaattaactattataatagtccctaatgtacttaatatctaactgcaaacattgccaagttttcttcttgtccaaagtcacaacagttttctttaggaaattttccatacaaagtggggcacattttcactgggttctttaagatgagataataaaaaatgatacgttagaaaattttccttatggcatcagtcttctctgacagattgcagcttcataaaggcaaaaactcttgattttctttttaacccacataagcccttaataaaatacccattacatgtagttgaagtaagaaaagctggaagtgaggaatggaggaaagaagaaaagaggggcacaaaatatttcattaaatgttttaaaaatccccagaaattcagaatacatgaatataggctgacattaaagcagatgtacgtctcgtgcagaacacacagtgaggaaacaaaatggcaagagcaaagctaaagaaaactaccaaatttgcattttcattaattcagccttaaatgtaactttgtgtcactgaataagtgacttacttcctaaacatgacggtcagacaggaaatcacttttcttgtgattagcataatgcttatattgtttttttagctacagtctaatcattacaaagaactactcgttgtgtccatattaaaattaaaagtaccttgccataacaaaagcacattctaagcctttcgaaatgtcaacatattaaaatatattaaagcaatctctattgcgggctctccattttgttttcaaaataacaataacagctatttatattgtataaaatcttgcattgcaatacaatttacattctattcagaacttcaaagtatttaaatatattcaggcaatctctattaggaacctctagtttgttttcaaaataacaataacagttatgtatgctctacaaaaacttgtatggcactatgatttgcattctaatactgttattcacagagattgagaatttatcacaagttgatggtcctcaagtggaagagcaacttctttcaaccacagttcagccaaatctggagaaggaggtctggggcatggagaataactcatcagaatcatcttttgcagattcttctgtagtttcaggtacttgaaacacatacgtactttaatacagtaattactacttccagaaaaaccaactgggcagaacctattaatcagagggagaaaatgtgggcattaaggttagtaaacatttatattaaaataatatttttagaggagattaaattaaaataattcttggccaaaatgctttctaaaactaatccaatatcgatgtgttataaatatgaacacaattgcatctaattttaatttataagaaatccatagtcattgtaagataagtataagaatgccttatatttcctggttttcaaagtacttataaatagatcagtcaatttaattcacccaaaaccctgaaaataaaatagattcattgctttttttctctggtttaaactgggatggctcagagaggttcaactgtgtcgtaaaaattaaaatcaaagctataaaaaggaagatgaacagatgctgaactagggttggcttgaattgcattaactaatcggtgaggactgaagactaccatgtccacctttttgatactgaaacagccctggcagaaacaaattatttaattattttttccctttctaatctgctcgtgataggagaatccacctctccccttctctgccaaaaggacttattctgttttcacaagaaatagttgcttaaagcctgtggtgtgctggtgaatgtaacaagtaactctggggaaaagaaaaagccctagtttgtagcatttgctagtctctgtggtttaaatattctcattcaggcttatttcaagcttccaacataagatcactgaacacaaagctgggaagagctgtgcagtggcacatctttatacagcattcccaccgtacagatgcaaaagacggagatagcctccagatcaaagatgatagttaaatgattaggtgatgagttttaaatacttacaacctttgcctgaaaataatttcttttatggtaagtttatacaatttaatttttgtaatggctgtggttaaaaactcagaaaattcctgaaaagttaccagttagctcctgtgagcccatataaactaactctagcacaccactgcaaagtttattatttttatttttttaatttcattttattttattttattttatttttggagatggaatctcactcagtcacacaggctggagtgcagtggcatgctcttggctcactgcatccttcacctcccgggttcaagtgattctcctgcctcagcctcccaagtagctgggattacaggcacccaccaccatgcccggctaatttttgtatttttagtagagacagggtttcaccatgttggccaggattgtcttgaactcctgacctcaggtgatcccccccactcggcctcccaaagtgctgggattacaggcatgagccactccgcctggccttgttattttgtacttagataagtaatatgcatgcattgcaagaaatctgtaatacacagaaacacacaaagaagtcaccaatattcccagaatgtggatattacacttaacatttgggtatgtgtgcttctgggctttttacatacctgaatgtgtatatatagtgtcatttctcttatgttcattaaatattcttctacagcctggtttttaatgactaaataatggtcccaccatttggatgcagtatgattaattgaccaattgttgaacatttaaataatgcctcgatagctctctttgtacttatgtcacagtttgctttttaaaaacatattatataattggaatttctgggtcaaatgatttatactattttaacacatttgatacttaatgtcaaatctcactccaggaacttgtaccaatttacatgttctccaagagtatgggatatgagtgcttatttccctgaattctcttctacgagaatgaataatattatcatgttcaatcttttcaatttggctgctattaaaaatggtatttcgttatttgaatttacattttcatatttctgctgaaaaagaatttcacaaattcattctctttgttttgttaattatcaatacttttaaaattcacttttctattagggtattcatcttctttttgctgacttatatgaactatctaagtatataagatatcaattcattgtcataaatagtaccaatttttttcacagttggcttgctttttagcctgtttgtggatttttctctttttctttttttgggggttctgggggcaggcggggacagggtcttgctctgtctctctgtcgcccaggctggagtacagtggcacaaacacagctcactgcagccttgacctcccaggcgcaagtgatcctcccacctcagtctcccaagtagctggcatcacgggcgtgcgccaccatacccagctaatttttgtattttttttagagaatggggtttcaccatgttgtcaagacttgtctggaaactcctgggctcaaggaatcctcctgccttggcctcccaaagtgctgggattataggcttgagctgctgtttgttttttaatgtaatgcgattccacctttctcataaactctggtgctgctgctgctgctaatagcaaagcttatacaggatagttttttttgttgttgttgttgtttttcatttgagcagctgcaccttcctttagaaatcatgattctataggaatgccaaaattcactatctgagtgtggagttttcatgttttaataaatgtcctttacaagctactcgacatctctatgagttactggccattttctgtaaatccacactagaaagaagggaagatggagctactgctgagagatgaccctgcatcttaaatgagggtttatcatcaaagattaccagactaatgcattgcattagcttgtagttaatctgctctgaacttctagtgaacaaaattaaaggcaaagttttaaatttttttaaatttttacttcttagtataaaacttaaagatgtatcttgtgaaaaaaattttaataatgatattaggggacagaaaagctttttatttttttttttcctcttgggttccttcctctctatctccctccttcccttcccttcctttcaccctaacttccttccctctcttcctttcacttctttcatttaacaactattttccaaactcctatcatgtgccaggcaccttgttgtatactgtgaactcaggaacataacagaaatggcttcatggccatggagcctaatatgactctatctggagattgtgatggtaagagataattaagagtaaatgagtcaggccaggcgtggtggctcacacctgtaatcccaggactttgggaggacgaggcgggtggatcacctgaggccaggagttcaagaccagcctgaccaccatggtgaagccctatctgtattaaaaatacaaaaacttagccgagcatggtggcagatgcctgtaatcccagctacttggaaggctgaggcaggggaatcacttgaacccaagaggcagaggttgcagtgagccaagattgcaccattgcactccagcctgagcaataagaacaaaaaaactccgtctcaaaaaaaaagaaatgacttcagcccccatggagctttccactgagggaggtgtcggcccagcagtagtaactgtatagctacacttttagtaagtgccatgaaagagtagtacagagtattaaaatatataatagtctgagagcatgtaagagtatacagtaaactgcacagtaatccgaggatccatccaattcacaaacatcttaatggtcttctagtccataggttctttatgatatctggatttctcatgtggatttgaaagcaggttagtttaatccattctggccctttccagagcactagcaaagagtcacttatttatgaaaatgtagtgtgtttgcttcagacctttgctttagactgctgctgtcattccaggtatctgtttgtttccttgttatcttaaggactaagaattactgatattaataacttataaagtaccattagtccagagaaattattgagttcttttgattgaagtttttgttgcagaatttactatggaagggttttgcctgcataggcattttctgacagaagtaacaatcaaggtaaatgtgaaagtgctccatagcttctaatatgcatttgtgtagttttgttttgtgtatcttcttgatattgataatgtttagtctttattttgaattccattatattaggttaaagtttcaaatagttgccaagatagcactgaaaaaaacttgtcacaaagtctgtaaatatgatttttatgtgtataaagaaaagactcaaattatttgtaattttaaattaagatatttataattattatttctgaaccagtttatcaaatattttctcagtgcatagtagtcttagtgcatgcaaatattaattatttatagctttaatgcaaagattatgtgtgtgtgtgtgttttagcaccttttaccccttaacacatctgatggataagacacactaccatcagtaacagtggcccaccttgaaactaattttaaacacatatccctttggcactagaggtgacatatcagagttctctagtggattgttctagagagtgatttcatcatatctccaaacttgcgggttgatatgtgtcctttttcctcctcccacctatgtttacaaatcaattgcttctcattaaaaaagtggttagtaattgatatatcaattttttatttgcccttttccaaattaccaaaacaatttctaaacaaaatttgaacagttctcagactattaatggcaatttaatgctggatgctatagttctgctttctgaagtcactggtcgtcttataacaaaagataactgcaaaactttggtgtttctctagatcagatttccctaggaaacatcagagagacactaagcctgctttcattttctattaaaagcattttaattatagcaaataattgacttctatatagcaaatattttacttctaaaagcgaacagttaaattagttgtgcagtcagtctttgtctgctactgctgctttgtcaaaacaagtgaatagttacttcacattaaccttttttcattatactattttaggaattaatgcattacaataaaatcaaaggaaattaagtgacatttaaaactaagtcaactttattgcatttggtataaacttttatgcagaatggagcagaaaatccccccaaacttctttaatcattgtgttacagctgccctattgtgaaaactagaaaatgcaaatgcctcagattggccagcagagggcgcctttacatttataaagccaatggaatttcactgtttcagcgtgaaagattataatgtaactgtagagtgtgccatgtgtgaatatacagtgcgcatgactgtgattgcatgtgtatattttgatttatatattcgttcacgtgaagattttaaaattccaccccagctaatgagcttaccaacctgcttgttttcagactttaataccttctttctcatatttccccatatgttccactccttaggtttatccggtgacattatgctttggaatatattgtggatatattttgcatagatcatgttctgtaggaggggtttaaaaattgacctttacaaaacagccttcttctccctggtccaatctgttatagcactttttttgttgttgttttgttttgcctttttatagactaaagtccaatcttagatttggtctatactaatttctgtaaaagtgccacaaaatttatccttgcacttgaggaccagaataatgttctgtagcactaacctctagaaatctagagatttttagaatctctagaaataaaaccatagcatttgaaacctaagtggaaaaaaataattgatacagtagtttacaataagactgtttcagaatcattcatgctatatcaggactggtttgaagtaagagcactcctttactttttaatttttctaaacttagttatcattaattattgagaactagcagtactactgaggcaatccaaaagacacatattaatttgagctgagagaaagagagagatgctgatgctgacactgatattggtacttgttaaaggattatgtaagtttgtattttatttgaaacctagcatgaggtatctgggtcagttatatggcttacagttgcataatagtcctaggaggattaccattcatacctcaggaatggggacataaatattatgacagaaagacagaaagaaatctccccagtgtgcagttaatacatgttggatttattttcatgatatctgtttatgactacaaacataaagctgtttttttagcagagttttaacaaaagcagtgattttaattttgactatcagatcttcccttcacaattttaggatgtgaatgaggtcagaagccagagaaataggaagaaggaggtgagttggtcagataatacagtatcattgagagaaccaagcactgaggaattgcgttcactctcaaaaagtttatactctaatgaaggaaaacaagtgacatgcctccacaaagaaactgtaatctagccatggtaaaagagatgcaagatctatattacaggatgaagagagaagatctgtaaaagaaggaagactgacgagagaaagcttaccagatgaggaatatcagatatttcatctgagccttgagagcactatgctggatttgtaaatagaagtggcaggcaacatttcaggtttcagaggcatcatgagcaaaagcacaagcacaagagagctcaaaatgtgttcagaggaacagtctgatgagagaggagcatttgtggaggagggcggtaggatttaagacttctaagatcagtgaaagttaacgagtttctctaacagtaatttttacaatgctttttctcacacacacacacttttaaaagtacaccatttatcatttggataattatacaatgccagcacaatgcatgtgtgtacaagcatcatctcagatgatgattgtaggaaagtccttaggtagaggaatatatctagcagagcgctggaactggatcttactggcttgtaagaccccagtgtgcacatctctttccagctccacacaggacatcatgctggaaattggccacggtgtgaatatttacaccatggaaattgacaaaccttttaaaaatcctgtctccccccaccaaagggccagttgttgaacatttacctgggcacccttagtgtattcgtatctccctccattctttcatgtctgcttaagtttgcttaactcctacagcctttcctgaaaagtgcttaccagccaaagaggtttctaattcacattctaccttgccgtctccaaaccttgttattgccaagatttacttcccatgtggttacctggcttctcactattaactgactctactctaggactttttgcctccattactgactggcagtgaatgaatgggtcattctggtgtgtgttgttatccttgcaacctgtgctttaataactacctctctttattagaacctaagtgcaaaggctaaagggcatagatgatacaatctaagccaacctcattacacacaattcaaatttaattctgttagaaacatttcagcagtagaaattctataaacaggagcagaggagctccatggatagccatgtgggcctgtggctctaggagagtgatttttctcgttggggaactcatttcctagaagcttggatccaggagagaatatgcaaggtgggtggaggtggcttctctccattctgtgggaacacatgttccctggcacggagaggaaaatctgacagaccaaaatgtgagtgtgtgtgtgtgtgtgtgtgcgcgcgcgcgtgcgtgtgtgtgtagatccatatacatagagctgaaaataaaagtgatctatttcctctcaaaaaagattaagttatttcaaaaggaaaggaaatatatattattagcagtatgagtgtaagcccacttgagaagcccatgcagtctttgtgtgtgtgcccaaacgtgcacacatgtttcaagctttcggggtagcttggaccttccaggcgtgaggattatgaaactggagggaaggtggggatagctgcaaaggtgaagctaaaatgcatcacaaggtcagaactagaataaacatgaaattaccatgaaaagtgaaattggactaaagaccttgtcaccactgactcgcaggagaacaggagcagttggtgctgttctttctcaatatgcacagaggccctttcccctcgcgtttcctgctgtaatgcaaaaccacttttcgggaaaaatagtgtgtggcagactgaagccaggggaaagaaacgaaagatattctgtggggtgataaatgcctctaggccaaaaatagaagaggtactacctggtaggaatagaagcaagaatcaatttgaattttacctaatggagcttagctctttgaataaagaatgcctacttgtccactacttgccatgttagctttttccagcagagggcagactttacattgaaatagatctaaaaaggccttcaacctcttgctttatttatttattttttttggtttgaatattttaaagttcaatatttttggtttctaaatgatttaaaaatattttaaagtatttaaggtctagagtatgttaaataagaaatttcttggatttcagttaatatttctgccttattagcaaaatactcaagatgtgattgcaaaataaggctttttttttccatggcttttgggctggtcttaggcacttgctgtcattgtaggcaaggctgttcttttttgtgttgtttacaaaattatgaaagaaaatcctatctcttaactggatggttataattttcattcattccaatagcttttgatataaataactgcttttctttaacagttcaattactaccatcttctaatctgtttcatagttagctttgatattttccctagaggaaatgacacctatataaaacttaactaaacttattatacaatataacttcaatattcaaagcccttttaatgagaatttaaaatttgtttctcttcgagtttcattttcttggtctaaccgaatcattctcacaataagtgagaaaatagagactactatctctctctatatattttttaaaacagaaggttaatttgcttgtaagtcgtggctacgttcgatctggagacttatctgttggactccaaagcacaaatttttttcatagcaaggcctgcctccaattaactccatgcgatctacttataaaatcataaacgcaggactttattcattaggacttaaatccagactgacatagtttgaaaaatacaaagaacaaaacaagataaacctaaataaatcaatttcagaaattcttggcatttaaactcaataggtcggtttaattttggcttcacgcaaacactttcttgatctcacttttttgggagtgtgtgtgtgtttgtgtgtaatacggtttttaaagtgaatgcaggaggaagatctctggggaaggcattctccctattataagaaatacaatatattttcttttattattaaattttacttatatttctatgaaagcaaaaacgtatatcaaaatgcataagaatttgctgcctaggaaacaaaattatttttatgtggagaaactgaagatttatatacccttaaaatgacaaagaaatcgagctccatatattcactttcattttcataataatctctacaggattactgcaattaattttatgtaacgagaacagaggtgttctttttatttgagtgcagaaaaaagtttaaaaagaggtgtggacacttaaatgtaaagttagtgaaaatctatcaatgaaaccagaaatgttagcaaagcaaaatcatctctctaagttcttttcttggaaagataagctctgtttcaagatggatagaaatgtgacaatacaatgaaaatgtctcttgctgttgaaaaacaccaccaccacagctcttaattcagttaacactgtgtttacaaaacttcacccaaatcatcagccatgcttagtgttagcggaaaaagcactggaacactggacttggattcagaaatgaggattctgccccactgtagctgtgtgatctggatatatcagttaacctctcagcgccttggtttctcacctatgaaacaggagataatatgtatctaccacacagaatgttgagagattaaaatgaaatttacattacatctgtgaaaactctgagttcagtgtcttcctgacacattggtgattggtttgttagatcaaaataatccagataatagactttattaataataccctattttgggtagcaacttttaaaacaattcatcaattattcgtgcacattgcataaatagctccctcccaaaacttacggatgttagaacaaagcttgggagaaatcttgactctacatgtagatgaagtaggtttaagattaaagtttaactgctagtgattagaaaatgaagtggaaatcaaatataattttcatttttattatacttggaaaatttaatgatacagtcttagattttagatatttgttagtattttttcagtaattttggtattctcttcagttctcaatattacatttaaaacagcaaatgaaaagcatgtcaaaattcacttttttgcctcaaaaatcttagcctatatttctagatctagaattactcttgcaatgacattttccccatctcattttacaccttgccttttccttacaaatcatattctcttttcatcagtacatctgcccaaaacgctcttctacttgacagttctatcccacattcgcctctgagcttctaacaagacatacctctgggaattgcgaggtgccatattttaaatctctcagtgagatatacaggttttcccgtgtgatcataagggattcctcaaatctgagttttagaactaatctgcgacaactgataagcacagtattttcacattagtatatttatgttgtgctaatatacttaaacagtatgtatgtccattgagaatttttctacaaatttcttatcagcttacttttaatttttaagaaagataccttacatatttgattggcattggctgtaattctgttatagctcttaaactaaataggctcatccaaattggatgggagatgtaaatgtcacagattctcagtggaatactagtcagcaataaaaaggaacaaattagtaatacacacaacaacgtagatgaatcacaattactataaactaagttttaaaagacagacataaaggctacatattgcatgaatccattcatatgacatcctggaaaaggaaaaactattggaacagaaaccagatcagtggttgccactggttgtctgatgtggaacagaaaccagattagataaccagagggatttactacaaagaggcttgaggagacttttggggtgctggaaataccttaactgtgatggtagttgcgtaactgtatacatatgtcaacctcttcaaagttgcaaaactgtatacatttgtcaaacttctcaaactataaacataaaaagtaatctttttagtaagtaaattatatcttaatatagctgacttgaaaaaattaactacttaaggaaaaaggaaatatttgcagttttgagttgtatatcatctcttttcagagaagttttattaaaacatactatgtgtctattttttcatccatttttatttccagccataaatatgacactcagactagttttaaatagctaagagctacctcattggtgtgttaagaggctttaggatgaaatattatctaagtataaatattataaaatatttttaattaaaagaagaacattatctaatggaactcctatcctaagaatatatcatttcttgatttttatttcattttctaattgccagcaattaaagtcctgtgccaagagcttgttgtacctaatattacttaaagattatcaagggctgtgaagtaagtgggattagcctcactttaaaacaaggggaacttgttaggttcagagcaaagtgaatagcaaattttgcaagtccttctgattccaaagccaatattagtaaccaccattgctttatttttaccagtttaacgatagttctggtgcttgaggatgaggttgtgtaagtgtttggaaataaactgtaagaaaagaattcttctctgagggaaagaaggttttcaaaaagaattgatcctgagttctgaaaacagaatgtcaggattcataaatgaaggatgtgcactgctattcatccatattaaccataactctttgatagaggcttaatctaattatagtaaagaaaaacagttatttccaacctcacatgagcttctagtacaactgtgatgccaggctttagtgatattctttttgaacaaattctctttgagattttacatctatgtttatcaccaaactatcattttcataactgatcagtagatccaggtactttattaatttttgtactttattaattccctatcattttaaatgaaaagacctcaaaggtttctatgtattattttttgtttaaaagacatatcaaacctaaatagtgttttcttgggcaacatttacatgtagaagggcaataaaagttataaacccttaggaatttgctttagctctcatactggatgtgtagtactttaagtaggtaatatgtatttgaatcactacccagcagctgctcatccatagtggatttttcttaatgtagcctgccagaagcgcttaccttttattcataattgtctgggtgtgccttccccacttgaattgaactgtatgtactgtgtgctgtagaggtgtgcagttaaaatacgtttattctcagaaatattttaaaaatattttaaaatgttttcctttgcagaatcagacacaagtttggcagaaggaagtgtcagctgcttagatgaaagtcttggacataacagcaacatgggcagtgattcaggcaccatgggaagtgattcaggtactgcctaactgttgtgtaaaccttaaaaagtgtgaaataagacgccatatagtggaagcatgggggctgttcataccctccctaatgtatttatgcaattaaagcaattcttttctacagtgaaatagttttggaactttgccattcactttctttaaaagtgtaacttacaaactcttagaaccaaactctctaattttgatccagtacatatctttctggtctggcatatgccacattccatagagtcatgtcatctattttatttatgggatctgtttcaattaatttgactgcatttaaatagactgtattcaatttggtacatttatttacttccaaattctactttttaaattaaaatactttttaaataaaaaattgatactatcaataagagaagtatgactaactagaaattacatatgtaagtaaaagaagtcaagtttgtataagtatcttttgaataggagtagagttggcaaaatatttcggtcactaaatgaaaattttaccaggtttttcccaaattactttcaatatcagttgtatattccaccagcaataaatgagagtgcctcttcctaattccccacaccttaattattacttcctgttatcagttttccaatttgttagtctgatagatttcaaattgtttctttcttttctttgagtgtatatttccatgatgactactaagctaaaatgttaacattattaaatgtaggtgatgggttagtgggtttctattacatacctttctatatttgcctacctgtttgaagtatttcacaattaaaatgaacaaatcacaaacaaaaataaaatgctttgcaaggggccagctaagaagcattggaaaaatgataaattaagaaacacattactttattcttcatttgtgattgagttgtttcccctaagaatatattatataattcagattatcctgcattgtcagaagaattataaaaattgatatccacaagaggcctaaggtggcaaaatagtttatagaaagattgtcatcaatcctaaatgctttccaaattagattgtagcacctcttttttcatatatccttttatcacaatgacactcctgaatcccttagaacaaattagtttgaggaaacccaattcaccaaatgtcattcacaactttaatcttcatttaaaagggtgaattcattttctgttcaattcagtcagtgtttattgagttcttgccatgtgcttatttctgtggattatatttgttttttctatccttgtcataaaattgttcaatttaataattaacatttctattacatgctatgctagaactcaaaatagataatctagctaatatttaacatcattataggttgttttaagtttttggagatcatgctaattttcatttgtctaagtaagggcttttcaataatatccattcattcaataaatatttaatgtgtaccttctatgtgcagggcatcttgcctatttagagctgttaacaaatgtatttgctgatttttagtcctcaatgtggcatgaaacacatgataagtctgaacgcaatctacttttgaaaagcaaatacattttcaaaggcaaatacatatttgaagacaaacacatcagaacgaacaccacttgaagttctagatggatcagtacttaggagcaaagaattactttgcctagtgggtagtcaatttccgaaatggtaaaattatgaaattccttgcccacaagtgcctggtataagctaaaatagaaaccaattcaaaaggaggtttagataaattggtgaactgttaaacacatattgaggcactgaaaagtattatagaatttgcaattgtgttttaatctttgaagttgacatcaaaagggacttcattctatatgtccttcctcctcctccagaggtcatatatgggccataaggtgtgacccaaaacaatcatattatattcctttgtggaagtgttatgccatagtagacatgattaccggtacttttaaagtataaatattctaattatattatgagaggcttttttttttctgtttcccattttcagaaagatatattgcctctcaaaaagtggcaatgtttttggcaatacatattggcctgatatgtagaaagtgtttccttactatttcaagataatgcctattaatatttgtccagctggaatggtgtatttttctgtggttacataccataggtaaaggaatactttgtagatttcaaacacatgcagtgacagaccttttctctccaagaataactcagctgtcaaaatcaatgtgagaatgaaatttgccacatggtgtagttccaatagaatattattatttaagccttatttgattttcttttttgaccatttatatggtagaatataattttaaggtctttaatcaatacttgttgtagagggagggcacagtgttattcaaagcttgtgcatattttaactgctattttcatttaattgattatgcttttcagagaaagccaaaaacaaacaaacaacaaacaatcaaaaacccaaacatttcttttataaaggaaattggctcctggctcattgataccagcccagttctctccttctgctgacacagaagagaaaactctcttttcattgcttctgtggataaattcttccatccctgtgtcagaggatgtgataaggcaagaatcaggctgaatatttagcactcacccagcttttattctttggatgctagtctcacattgaatccaactctaattttttcctgtatccacactgtgcttctttttaaacttagggccacctatcaaatctcctagtcttaaatcaaaaatatggaagattattgagctatctgtctaaccaggtatttttgtgtgcataacagcggtatagtgtcctagactctaaagtccttcaggacaacaactatattattgagagtgcctggcatataataggtgtccagtcaatattgactgaagaataaatttgtggtacatgcctacttaagtagcagtaaaacctcatgacttcagttctgaagttaaattgtaaacacaatagtccttttaacattctcgctcttcttaaatgtatcagcttaataaatgattagaatgcatgagtaatattaatattttactcatttgatttggttctggattctcgggaaagagacttaggaaattgctatatctttctctttgtaatatttcttattcaaggatatagtagtaagcatgaaaaatgggggaaattattattaaaatgtactcttttaatagtttattctgcaactattatgaaggaaggagcattttattagtattcttaaaatctacgcaaaaatgaaactcagatgaaattaaacaaagaggatttattcctatattttaacaaacttcctagatcaaacaaaaatttagcagtagtaaatttctgggtctttgtttagacactttataatgcatcttcctttgcaaatatactggctcttttggaaaagttagatgacaaactatcctaatagaggtgaaatatcatggttttgatagtctatgaaagctacagagggctaacattgttcttaaataaatcagagccttacataaatataatacaagatgaactattgtaaatattgtagagttcaaaacaagcatggaaacttataaaggtatattttaaatgctggtgattttaaatgagtttttcataaaaatcatattgaatgaatcagcagatgatccaacgatagcttaaatgtattgcatctgtttttactcatccatacaaaaggcaaatttcctgccaggttagtagatgatcctaagtggtatttggtgtgaattttcctacctgttcagatagtactatcacaacaagttaaaactcactcaaatagaattatccaggggctgaaatggagccatcagcaagttttttgaagttaatgggattatgttagtttagttttattgcaaacattgataagaaaaagaagttatttcatgtgtgatgaaatatggataaatactgttatttcattttttcttcttattatataagtcataaaatgggcttttaaattggtataatatttatgttacttcatttcacattataagctatgcattttttgatctttttcccctggtaatataggtcaatatggtcacttctaaataaaatatattctatctaaaacaagacatattataataattttattttaatatacataaagtttatgttagaacttattttatcagtattttaaatttaaaatccatgttaattgaaaatatttttaatatattatactttggattttagaaaaagttggaatatgtggagccataaacataaggaagtaataaagataacaattcacaaataaatggcaaaagtagtcatgatttagtttaaaccaacaaaattatttttacctttcaaggaaaacctaccaattaaaggctagcataggaagagttttttcctctactctctaaataacacttctgtacaacgtagttgcctcagatggaaaaaggaacttgagatagatagggtgtctgtgtgtgtgtgtgtgtgtgtgtgtgtgtgtgtgtgtgtgctttttggtatttggttgtcagataaagtttctacaaacgaaagaaaatagtatcagccataataccttctttgcaattaaaaaaaggggaaatacggaattgggaggactaatgattccaactttctcagctcctcttgattttgtaggtggaagggctaagtgaaacaagcagggcatagtggctccacaaagaaatgggaagtctgatgccttaacaactagacctgccaatcccgcaccacagccctttctacaaattcgccttggcttcacccactctgtgaaaacactcccccatgttgctccttccagtcctcttaattagttgactccctctgcacccccacacccccagactccttctgtgagagggagcctgtgttgtattctgcagatgcagagagcagacattaattggtacagaaaagagaaaatgaattggtttggaccacatgtgaaaaggagtttaaagcacaagaggcctacgttcagtttcaggcttcctaagctctgttattttgttgtcccttctcttatttttcttttcaaccttcttcagatgaagagaatgtggcagcaagagcatccccggagccagaactccagctcaggccttaccaaatggaagttgcccagccagccttggaagggaagaatatcatcatctgcctccctacagggagtggaaaaaccagagtggctgtttacattgccaaggatcacttagacaagaagaaaaaagcatctgagcctggaaaagttatagttcttgtcaataaggtattcaaagaacacataacactagttgatcattttcattgtattattctgaaacttttatatgatggcatttgtgtcattgactcacacatgtttagtttaattaagagtaatgtcaaaataagctctttattttatctttaatcttttttaggtatatgtaatatctaagtgatgaacctgaaaatagaaatttttcaataaaactaggacattataccaaatagctataacttatgtagccccaacacctgggagttagttttgattcatctgcttcctttatttcccttatctggtcacaggttatatttacccagatatactttaaggttaactagctaatttatcattgcaaatgaatatgaatgaccccaaaatatcactggaatcaaggatcaaatattgttttttactttctctttagctgttgacccttagtcaaagatggcaagtcctctttctccagaatacatataatgtggtttggagataaaattaagtgaaggtttggtatctacatcaagttaaatagattttaaaatgctcaagaagtatacatatatatatataaaatatacacatattacattaaatgcaactgaagttttccttcaataatttatatcttagggtatttgaaggccatcttcataagtaaatatgtctgcttcaccgtaaaacaaaatctagtcatttgagctttattcagataaatgacagtttattaatttataaatatacaaattggggatatgaatatttactattttatggaataaaaatgagcatacttttccttgatcagaaagtcttttttctgttcttatctattcctttataaaaaaaagcagtagactgtaaaatattctattttttatttctattatgctaaaagttttagttacatcctgtttttcagtacttagaatccatgatttgaatgattcctatcatgtagttccaaatgcattttgtgtgcatctgctaagtccaaagagctctcctttatacttgtatacttactattaaaacacttttatttattttgctggtaatggtgacacattcaaatttgtttgccgatagacttgcatatgtattccctttgtacaggatattatttttttaaatgaaataggtttgaatcctatttctacctaccggtactttgaattagacaaattagtaacttctctaactttaatttcttcatctgcaaaatgaacaaaataataactaccctgctctgaaatttcaattggattatatatggcctagtaatgactcctgcttttactcttaaatactagctttctcctgctcctttctcttcaccttcataactcatgaaactaagtaacataaccagggaggcttttaagtgccaaatccaaagaaaagaaccaagttaactggattcaaagctacttaaactgataattttgaaaccatttgagcatctgccattttctcaaactcagcctcaccatctaccctcccaaaggctcctcttccccatttacctgtctctttgtgagtaataccacctcctccaaagcatgcagcccccacacctaggagttagttttaattcatctacttcctttatttcccttatctggtcacagggacataggctaatagagttgaaaaggggcttcgagattcagctttcttcctttgcagagtggttctctcacttctgcagggtaacttcctaatatcaaggaggtccccctactaacaagacactgacactcttagaaagccctatttgtattcatccaaatctgtttctctggaatcccacaatcaccggttagagttccttcctctgcaaccacacgaagtcctaattctccagcctaatgacagcccttcacatagacaaggcagcatcatgtggctcctaagttacttttttttgttgttccttggaataaatgtccacagttcctttaactgttttccatataacatggtttctaaacctgtcatcattcctagtgctcatactccttcagacatttttctgggacactcttaaatgcggtgtccagagttgggcaaaatactacagataagatcataccagtgcagagcgtaggagacatgagatatgacacccagcttatgaccgcatttgcctctggcatccacagacttatctggaccttgtcatagaatcaaaatcctcagttcctttttatttgaaaatctcttaaactagaacattcttaatttatttttgtgcagtcagtttctgagcctaaattcaaatacttaggtttatttctttaactattgatcttttcaagtctcatatttttgtatgtgtctgatgcatcttcctttctataatattcccttctcttctcaatgctatattctagccaggaatctttcattgcattggatctacatgattcttcggaatgcacatttatggctatgtttaaatattaatttatcctcattaattattcacattcactgcattgttgtgtggacacctggggacttgactctaattccttattgcttttctattgtttttctccttagaattactagaaatccctccactaattcttccatctatgtcataattgtttactataccatctttttgaattttaaattaatttcagttttaattctttttgattgagtcagaacacctctggcccagtaagtctaagtaacctgaatcttgtgtttggcccaacaacaaggtattgttacccttgatctagaaaaaagaaatcctgttctctaacaccatttactttcatctcccttcctccgtcctttccaaagttatagccttcgaatgcagagagaaatgaaaacaccacctgtgttcccagactttccatttcctatctagggcttcaacatcatgagagataaatcccaggttccttcagatgctgcaatgcagccccatatgattcagcagaagtgggtagcagttgggtttgattagtcttggcaggcactctgtcacatctcaagaaaaggctccaagaggaccacacacctcccaattagccatgtcagacttgcatctggctttctctgtgtccttcagtaggaagtcaacaccacccagagcgagtatcaggatttccagcatctgctatctcctgtgacaacttctttttgttctgtacatcagaaggtgcggcttcttttgaagatttttgactcaccttttatgggcagaattttgtggtgcaatgtcactccataatacagagattcttaattaaatcattcagcatgagtgtatttaaagacaaatcaaatgtagatcttgtcctcacgaaatatacagtctagaagaaagatgagagatatatgttgataatatgataattaatattttattgcactttataaagcactctcagatatattatctcattaaatctacacagcaaccccataaggtagtctttattattctcattttttcccatcagaaaactgaagtttcaagactttaattggcaattcagggatcatccagccaagaaacagcagaaatgagacacgaacttttatcttctgagtccactggtcttcctttgccactgcaccctccttctagaataaaaggattggtgaaatatcgtggaagttttagtactgacaatacagcatcctctatttcatttaattttcacaaccctataataggttaacattatcctcattttattaatctggaaattgagagcattaagatgattgagaccttgtccaaggtacaaagaagtaagtggctggagtagctgttttcagggctttcttccctctgcatcatgctgtctctttccactaaacttaatttccattcttttcctcagaagccttctgtcttgcataaatgaaatggaaagaatacttctagaaaatgattcaagaacggctttgtggaagagagagagaagagcagagctgtgactctcaaattttaaaggcttaaatgtcattaaagtgaaggtttcaagcctctgccccccagagattgtccctagattcagtaattcttgagtgggacttaggagtctgcattttctaaacttctcaagcgtgtttgtaacaggtagattagagagactttactttgaaaaaaaaaaagaactaaatgaaagtttctcttcttactctttctaccctttccaccttgtggtaaggtaatgttctttcactcaccaacttgattactgattttaagtccactttgcttccttagaatttatggatttcattttaactcttccccaccccttcttccagccccccagacacctacttttggaagcctccatcatttccactaaataataaagtaaaccagtgttcttcataaggtagctgttccctttagccagggggatcagccattccttatttaacacctaagtgggacttacctcaagtaaaaagatgaataacacaaaccctacagactaggagaaaggtaaccactgattgcatttctggtagaattatggagagtgaataatataatgggcattctttgaagcttgcccttgaaactcttcctgccaactctctgtttcccatattgtacactgctgggcttccattgcataaatcttcctaggatatgaccttcaagatggccttatattttttctcctaatccagaaggtattttgagtgactagtaatgtgtagcattgaggtgtcaacccagctaccttcagcagatgttataacatacagaatcagctgagaaggacccgaagtttccacttcttttgccaggtctactttcgggtttgttctttctcagccaccacagctattccagcagctcatttcttcacttcacatctgttctcctagtcaatgccctaactcctcacttgtggttccctgattcttccggtcctgtgggcctgttgcttctggtctaatccaaaacaaacaacaggaataaatcacttcaacagtacaacttgtagggatcctgtaaactgttatactacatgtaaaatactctgcttgatgtttgcagccctttataacctggcactgctctacaaatttaaaaatgtatttcttattcaaccttagcttcagtttatcttttcactgactcatggcttactagattcaaattccttcaaattccccttttttgagtcgtcagttgcttcttctcttaactgtcactttgtgtgtgtttctaggtgagcattattacattgtgttgtaataatttgtcttgctttgtttagtcttggtctgtatgatcagctagacagagttcctcagagttgtcaaccatatgttaaatatctttgtgtcccagacataaatgcccacacctaacaagggtataatcagtgcttattgacagatgaatgaaaggagtgatgggaacgttgttatgcctgattcactgataatgtaaacacctcttttcatgctggatgccaaacaggcctttgatcaacactcttttggcttcgactaactttctctcttttaaaaaatcttcacatttttttaaggtactgctagttgaacagctcttccgcaaggagttccaaccatttttgaagaaatggtatcgtgttattggattaagtggtgatacccaactgaaaatatcatttccagaagttgtcaagtcctgtgatattattatcagtacagctcaaatccttgaaaactccctcttaaacttggaaaatggagaagatgctggtgttcaattgtcaggtcagtttaagtgtttatttttttaaaaaaattaatagtaatattgatgataacaaaggctgtatttgttaattttttaaatgttcgtggcttaaattgtcttttattgcttatggttttattaaaatccttctacttcctggaggaaacaaaaaaagtaagtttttaattagaccactttattctgcataagaataccattattaaggcaagcactcgctggactttccatttattctgacaagttcctttatttgctattgaatgttgttagatctgcttccctttcccatagaataaaaatacaaatcttgtcttttatggggtataggatgagaaggtaggagaaagaatggaaaaaaataaaaataatttataataactaaatgcaatgttaaaacaaggcaaaattttccctctttaaatagtgtgtttttcacttgatgtaataaaaataaaattcaccaacctaaaattaaatattttattaaagaacaaaagtaaaatcctgagatttgttttgtaataagccagggtgtgaattccacatctaacgctttttagctctgtaaactttctgaatcttatctattaaagaaggagaatgtctaacagggagtttcatgattagtatatacgaacgtctgtgtgtacaggtgctagtatagttagatttccctcctaaaaactgaaagtggactgctcttgtgtcgtgcccaagagaaggcatccccaaggcagctcaattactggtctaaagctattttctcactgtagtgtgctgatatggagaaatgaactctttgaattttcttttaagacttttccctcattatcattgatgaatgtcatcacaccaacaaagaagcagtgtataataacatcatgaggcattatttgatgcagaagttgaaaaacaatagactcaagaaagaaaacaaaccagtgattccccttcctcagatactgggactaacagcttcacctggtgttggaggggccacgaagcaagccaaagctgaagaacacattttaaaagtaagtcataattttatgtataaccaatgaaagcaaagctaaaacaaatgccaggacttcttggtcttattaaataagattgagttcaagtgctgtgatcatcagttaagtgaaatctgtttgctttagtacaataaatatactcagtcttctacagaatactaatatgccttaataaatatcaataacacaaaatcatatcataaaaatgatggagagtgatataattaatgggaagttgagataaatagaatgccttcttttgaacatctactcttatggttaaatttaatgatctacctgatatagaaatatgcataaactatgaaagtttctagttgtcacacataataaagtcttactccatgagtttcaaaactgcgtattttctgtccttcatatgttattctaggaatctaggtaattcaaattaaagggaaatagggatatgaagatctttaaagatgagcttgtcagctgccccctaatggtaacaccacatacagtgctactgttaatcttgagtgttaagtaaatcagagtagcccaagaagtattttggtaccaaggcaattgcatttgaattttaagtcccttactttaagttgaattcaatacaaattatgtagagtagacacaacattcagagatgcaattataggctccaaaatataatttattactgggattaaggactaattccaatctaataatcaaggaacatgactaaggatgctcccagctttatgtgtagatgcctgctttatgtgtagaagaaggtggtagtatgatccacggatgtcaagttgacctttgatatgcctcatgtaacatggttatctcaaaatcattctctgtcaatgatggtaaattgttatactctttccatgtcagttttccacttggaaaatggaatttaaggaatgaatatcaatacacctttttctctgttattacaccaacttgatttagaagctcctttttcctatattaggacaaatgttgctgattgtgggttaatgagatccataataaaacaatgttaaaatgctatatttagcaagcaatgctacatattactttcatgaaattttataccacatatttatgctacaatttaatgctacatattttttacatgaaaaagagttctacgttgaataaagtgaaagggaaaaataattgcataaatattgaaaaatgtctcttttccagctatgtgccaatcttgatgcatttactattaaaactgttaaagaaaaccttgatcaactgaaaaaccaaatacaggagccatgcaagaagtttgccattgcagatgcaaccagagaactatgggcttaatgttttatatctattgattgacaatactacattcagtagaaagatggcaaaggctattttatactgaccatctgaaaaatattatttaaaaattttagcaaacaagtttcaggtttttttttaattgatgttaggcagtaaagcactacagtgaattctcgcctctagacctcttttagttttctaatttacatgaggaatagcttattcattgtttgtacatatgagttaaaagggacagtgaggggccatgggcagagggatgtgtaactaagctggaggggttatgacagttcgttttagcctttttaattttcttcgtgaattccaggattgattttctgacattccacagagccattttgttgctatcaaattacatttggcaatgataatgggaatcctatctaatttgcatacttgcatatttactttcttaggaaaacagtgcaatggtaaagaagtgaaatatttactttaatgatgcttttctgatgaatttttcaggaattataagaataaactgagagtcaatttggacattatggccagtaaattatgtcaatactttattttgtaaaatttagaaagcattacaaattctggccatatgtacttgataataccattgaaaataagaatgttgggatcaaatgatatgaaatatgcatagtagataaaacaatgcttgaaatatagtaggcactaaaccaatgttagtaactagcctccgtatatctctttttttaaattctgaaaaagactgctatctactccagctgcactagcacagttgaatatattttattcactttcctttttttgttacttatttggttttacttgataattttaaatgcatgtttctactcataaagatttttaaaactaaatttctcaggtacattttttaaaagaatttgccattttttaaacttactttaatttttttatataccttgaagcataacatacatacagaaaagtacaaattcatatgtgtactgattgctgaattttcctaaactgaatacacatatgtaaacaaaatccaaatcaagagacagcacactgccagtatcctaaaagctcctggctcccccttccagtcaccatgccccaggaatcactactatcttgacttctaaaacaaaaaatgagttttcactgattctgaactttatataaatggaatcatatagcatgcactcctttatttctggctttattcagactctacttcaactttttattttggtcgaaataatactgaaaccagttttacaaaggaggaatgtttcttaaattacactgtaagctttctaaataattgtggcattaatgcaatatgtgttttctaagttacctttaacatacaaggtttatataattcattaactttacttttgttaaccttttaagttggagattccagcagaggtaacagggtgttggtgtgggtgtggcttgatggcaggcttaaaagagaagctgacaaaaataaatgtacagacaagtgatgggacactaacctgttactttgtctaaacaaagatgaatcagatttattataacctagaaattattctaacaaagataatgttattcctttaacaataagaatacataagaataagtgtgtactttatttttaggatccatttaaagagaaacttctagaaataatgacaaggattcaaacttattgtcaaatgagtccaatgtcagattttggaactcaaccctatgaacaatgggccattcaaatggaaaaaaaaggtaatttaggtttggacctaacacttagtttatcccatgtttataccaagattttagatgactatatcacagattccccaaaaaatggaaagcaaaagtagttccaaatagaatttctgtgttctctgtaggacaagcataataaacatgttctaattccttttctaaggagagagattccaatgctatccaattttctctttgtctccagagtcttgagataaactttaatagctctgtctccaaaggcaaactcaactaatatattttcatgccatttctctgtgcatcagctacatgtttttgctgaggcagaactcaagcagcaagtctaggagttggaaaacttgccctgtttcagagctcaggacacattttgagccttaacttgcccattttaaaaccaaggtgaagccagttttgcagttagtaacatatattcaagtccatggcacaattttagggggtttatatgcttattatttcacttacataatatattttttcacagctgcaaaagaaggaaatcgcaaagaacgtgtttgtgcagaacatttgaggaagtacaatgaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttggtaagattcaaagtaacatatactggtgtcattcatttaccttttcaacatgaacttactgcttctcataagctctccaaaaagagaaatatcttttgaaagaagaaatcccactcagattggaactatttctaatgatatgaccagattagctaccagttgtatgatcacagtcagtcatttggtcattcagttttattgtcaagacaattgtaatgaatttatttattaaaccatcctaattattagatatagcgcaagattgtagatgagccacaattcatgaaaccttctagtgtaattcgttgtatgttttactaatttatacttccaaatagtttttaaattaattaatttttaatcaataaaactgtatatttttggtgtacaaaatgtttgatatatctgtacattgtggagtggctaaatcaagcttgttaacatatcaactctaattatttcttaaagaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacaggtataaatatccttttggacgacttcttttcttttctttctcttttcttcttctcttctcttcttctcttcttcttttctttcagacgaggcctcgctctgtcactcaggctgaagagcagtggcatgatcaagattcactgcacccttgacctcctaggctcaagcaatcctccagccccagcctcctgagcagctgggactctaggtgtgcaccaccacacccaactaatttttttatcttttttgtagaaaaggtgtcccactatgttgtcctggcttgtcttaaactcctgggctcaagccatccttctgcctctgccttccaaagtattgagattataggcacgagccactgcgctcagcctggacttcttttcttatgctgttattactcttctcaaattgtttttagtctttttctaccaatcctctcaccacccatgaatacatttacaggataaaacacctgttcttgaaataaaatctaaggctaatagcaatcaaatctgtggtttttgagaggttaagtgttgttttaggagtcatttttcccacataataattggaagaactttctaagtttcccttgcaaaaataagtgacatgtaagcttaacaacaaagttcagaaatgattgccatgtagtatcaagtggaaaattcaaactcccacaaaaatgtgttcgttataactagaactatgcaaaatatatttacatagaatgagacctctagggaaacttgaaaaaactaaagaatttgatatattagagatttgggattctggtaagtaggttttctgttccattttaaggtgttttgttttactttgtttacatgttgcttactgtgggcatagtaaaaaaacacttgggtaagaaaaaaaggcactagtagacattacaagcatgcatgaagcaatgatgccttgttagagtaaaatattcttctgagggctaaaatcacattgaccctgagtctttctggacaaatggatacttcctttctattatagttatcactattcaagccaatggagcaaattaattctgtttcagtgtttgctggaaaaaaattcttgaaatggttcactataactctttatataccctctacatgcactcctgtgagcattgccaataaaattaatgccagagaaagtactgttttccttgggtaacaagtttgcttccttctttttgatgagatgtagtttgctttatatgtttttctttttgccttagcttccagaaagctagcaggaaaaactcaagttcagtctcacagcctctgtgttgattcttatgagttctgtttttaaatttctattatattaactttagcgttgtgacttttattgtaagagggctaagcttctctgtaaaaagaggtagagtgcaaatgaaaagcctatgttcaagctactttctgctccttattcatttcaaaccaaggggctgtgtgccaaaatacagaacatattcccttccttccccactccttgtatcctcaccttgtaaactggggtggacaaactccttagcaatggtgttaggcatctatgtgatgtgacttgaagatctgacactgtctggaatttttgttattaaagttactccttcaaattaagacacaggcatcttctgctaaaatccaagaacatgtaagtttttatcgggagatgtgagggccggagggagaaattttcagagaagactcaagagaggcctttctacagggttctccgacagttaggagtaggagtcaaataaggactcacactgcctctgagaagaaagggaaggggatggaagaacatacttttggtctggcagctttatttttgtttaatgtatgggggcaatagacagtgtggatgtgcaccaaatgccaactggaccaacagctttgatgagagtggttatgaaaataaaaccacattgtccatatcaagctaggtttgtgctcactcatttctcccatgtaaggatgagaggtcatattttaatttatttatattgactttcaaaattcagagttttttcatttgaaaatagttttatttacagttgcataacctcttctttcatttttatttatattgtccctttgaattgataatatatttacctcattcaaaattaatgaggcacaaggcataccactgacctctagtcttccagctcccctccctagatgcagctgatgttttcagtttctcttgtgtccttttgggaatattccatagcttctaactcaaatgattcttaaagtgaccaaatgcatatacagtgtgcattttctctgtgactgaaaacaatatatcaaagcatctctgcatcttatcctctaaatagcaaagattctgaagttaatgtagtgggggaaaaaagcccattttagcttctgattgtcatatcctcttctcaaccgacagctcctcctttcttcctctggagtcacccatcaatttatttccttggcataaaatgtggtcttcccacctccagactggatgatggaaatatttttgttattgtgtggtaatcccagattggaaatcaaatgttgtctcaccattatgtctttgttcaatgccaaaaacagatggcaacaaatccatgtatgatctaatcctttgatttacactgaccagttgcaatttattatttcttcctctaattagaagattttaatgtgtttagcatcacaaaatttaatttattaaaatgtttctacagaatgaacaaaaagaagtcattagtaaatttcgcactggaaaaataaatctgcttatcgctaccacagtggcagaagaaggtctggatattaaagaatgtaacattgttatccgttatggtctcgtcaccaatgaaatagccatggtccaggtacattcaaatacctctacttactatgttaatttttatttgttgtgttcctattttaattgctttcttttagttaaacagcaaaaacatctttttagtttttaaaacaaagcctttttcctgttaaaagagcatttttaaaatgctgtgcactgttgcctttcatattgctgtcaattcatataataaccagatacctgttgtatgtttaaacaacaactcctctttagctatttctgtaccctaaggctttccaggaccatgacactagccccatttgtaagagtcaaaagcagcctcctctttcctaggatgtctgtgagcccccagctggaccaggaatctaaagtacactgtcccacaaatgtctgatgaagtaacatgcccaaacctgagggagccctgctgtttctgcattttcttcccttatcctttccaattttctgtgtgtttagaagttttcatgataaaatgttggttgggggcagacttataaatgtaaaaataaataaaagagtgaaatgttctgttttcattcagatttcactttatctcttacccacctatgttaatggcattattgtaacctgttgagtaggcataggtgttatgctgattaatagatacactattcttcctataaaattaactaaagggatttttaaataacttaaattgcatccttgaataaaaggcaagtcatagaagactaagataaccagactgccatcaaattagcaaataattttatttcaggagtctatttgtaagtcagtgataagcttagtaatcaaacttagaatacatttttccttgaggatacttttatagttgataggctcctgggccaggccacagaagtttctattaatcaaaatatacctgaatatcactttcataaagaggcttgtagaaagttatcaccatgttaaatatgttcccatgggaaaatgtgctaagaatgtaatgtaggatgccagaaactgacctgccttctcaccagccctagtgtcaaggtcagagatgacccttctctccattcattcctcccagcttcctaccaagaaagacgactcctcagtccaagtcattgatcgtggttctcatagtttgagagagaagttctgagagcaaaaagcctggacaggatgatattatgtcattgaggcctaaccatcatcactctagaacactttttctgtattaaagcatattataagtttcaaagagctgacttacaagtaatggattaataactaactttactaactttacccatttgaagactggcatgctgaacaacccaatatcatttcattctgagcaagaatatcattcccagaaaaaaccaggaggatttgtatcacaacgtaccctttcatttacttgatttatttgtgttttcaggcccgtggtcgagccagagctgatgagagcacctacgtcctggttgctcacagtggttcaggagttatcgaacatgagacagttaatgatttccgagagaagatgatgtataaagctatacattgtgttcaaaatatgaaaccagaggagtatgctcataaggtattgttggtcacctctgaatttgattttcatttaaaaatggcggcatgtggttgccattgatatctctgtgactcattctccagacaaaaattgtgctgaattataagtttctgtttagtgaactgtgcttaatccagattcacatgcagagaatcaagatccatggtgggcaggaaattatcagggagccattcctcattccagtaaaaccaaccataaatttggataatataaatttagcctgccttgttccagaaagactgctgcaactcactgattcaatccaacatggtaattttggtttgatccaatttgtttgtaaaggcacagttttttaatttcagaaatgtagatgaaacgcctactgtgtgccaggtaaccttataggaacagccaacagaggaaacaaaacacataagactcctgctgcacgaagtttatatttctagtgtgtgcctggtgaagtttggaggacagaaacagtaaacaaacattaaaagacaagaaatataatatatcaagataagtgaagagaattagaataggatgatatgaaacaactgactagatggatactttcttgtttttagtagtttttgaaattcttttttttcttattatactttaagttctagcgtacatgtgcacaacgtgcaggtctgttacatatgtatacatgtgccatgttggtgtgctgtacccatttacttgtcatttacattaggtatttctcttaatgctatccctctcccctccccccaccccacgacaggccctggtgtgtaatgttcccagccctgtgtccaagtgttctcattgttcaatttccacctatgagtgagaacatgcggggtttggttttctgtccttgtgatagtttgctcagaatgatggttttcagtttcatccatgtccctacaaaggacatgaactcatccttttttatggctgcatagactagatggatactttcaactgggtggttgaggaaggtctcttagaaggagtgacagggaagctgcatctgaatgataagaaaatacagaatatgtcatctagctacatctcaatttactaacacacagttatttgagttgtgcaaaggaatgcagtgaagaaagaatgtatttctatagtatttcaggaaatcccccataccactgaattaaaaactgtaactactataatagcaaacaatccatttagaagaaaattcacacccttgcatatgggtgtgaatgttagtttcttaacattggaattggctctaccttaaatagagtttcactaaagggatgctttcaaattggtgtgcattgctattaccaactccaaggtttcctgaccaggtttctgcacaaaactttattttaaaacccctttaaatacaaggcaaagcaaatattttagagtatcaaatcaaacttttgtcaccaaattatgccactggaaagttactgcctcccagttaatgtcctaagagaattaatcaagtagatgtttacaaagactgatgttttaagagtagttttttgggggtcaacactaggacaatctctggagtttaagggtcaaattcttccagacattctttagaataagactctgtccctttgggtgccccagaatggactatatttttaggtctcctttggccaagaaacctgccctcctaaaactacttcaggtagatgaacttagagtaggagtggcaatggggaaggagggctacatgccaccagtaatctgttcaaagcctgtagaaaacattgctactgggaagatgtttactttcttctaatttgagcatctttctcaacacaatcttcagacaaccactaatgaatgatgagaattgacagataagatgaaatctttcttccatgtcataaacacagtttaaacataacagagcttcttaaaattaaatcagacttctaggagcaagttgatcatcataaatgtttttctctagcattgattcaatttttttgctacattattacactttatttggacttaggctgattgtagctgtgcctcctgtatatttattatgagttcttttttttgtttgtttacccctctagttacttcaaattcctttactattttagagttagtcttttagcttctgatgtcctgattacaaatccatacacttaccttggctttattttttttctgactcattcatacacatttcttaattcccacatgtatgtgatcctataattaccttatcagctcattgaactgtaaatgtcttataaaagtaacctattaaaacttttagggaaaaatatttcagtgtattattttgctataatttacataggaaattaatcttggtacttgtctgattaatctattatatagcctccaggcttgcttgcaaggttttattctcttttctatgctatatatggctgccttgaagggagatctaaaatgtagagttagaagccaaagagatgggctttgttcaactgcactgatgacttacttttactgaaaatttctccctcggtcttaaagaaaacaattaattcttccataatttaatataaatatgtcatctatgagaggtcagtttggcggtaaattattcaagaaccagatttttctaccgtgaagttgtgttggtataaacactgttcagtccctatgtcaactttcatatgttacataaactagattatttccattataaaataagtgctgctgctagtcacagagcatagaataaagaggttgatgggcatattctctcacagaaggaatctaggcagtattggctccatgaagttatgaggaatccaggccttttctctcctttttatgctgttatagcattgggctttcattctcatggtcgaagatggcttttagaatttcagctgccacatcctcattctaggcaggaagtaagaggagaatggaaagggcgaaaggtacacatgccagctatctaatttttaagaagattttctgaaaattccactcaacagctttttacatgtcattgactgctggtttggaaatgtagtcatttagctagcatatttccacccagaataaaattatggttctgttaggaaaaatggacactgaatgaaaaactagtggtttctgctacaaatgtgtaaagagtagactgattaagatggtgaagggtctgaaaattataaagcatgaaaaaagattaaaggcatttgggtgcttagcatagattagagaagacttggtaatgtaataacagtgtttagtaaagagattccatgtggaaggaagtttagacctattttatggaacagaattaaaacaaatgggtaggctggcgcagtggctcacacctgcaatcccagcactttgagaggctgaggcaggtggatcactgagatcaagaatttgagatcagcctgggcaacatggcaaaactccatctctactaaaaatatgaaaattagctgagcatggtggcaggtgcctggaatcccagctacttgggagtctaaggcagaagaatcccttgaacccaggagatggaggttgcagtgacccgagatagtaccaacacactccagcctgggcagcagagcaagactccatctcaataaataaataaataaataaataacaaatgggtaggctagacagagaatttttgttgaatattgctaaggactttctcacaatagaacttttctgaaatggaatgggctactttttgaatacctgaagactgcacattttcatggttacaggagatgattatataccaaattctttggggggtcattacctcattatttttatttcctagaccataagctaagggtatattttcacaactaagacctccaaatttcaggagacttacttctatgaaactgaaaaaagaaaacaaacctgaaaccactaactattcctttctctgtccagattttggaattacagatgcaaagtataatggaaaagaaaatgaaaaccaagagaaatattgccaagcattacaagaataacccatcactaataactttcctttgcaaaaactgcagtgtgctagcctgttctggggaagatatccatgtaattgagaaaatgcatcacgtcaatatgaccccagaattcaagtgagtccagaaagacccatttttacatttttccacaaggggtgaagcaggttgcattgtaaatgttctttcaaaatagtgcaaatgagattcatttattttacaacttcctctcctttagcctctcaattatattcctattttaaatgtctatgttttaaataaactgcactggggcaggaaaagagaagtgtaagaaccaaagccattttgatccaaggcacgtatttttgtatgctcatcatttgttctcaaatttttgaacaactaaagtcaagttaatataaccaacagacttgaaagaagttatgaaaggcaacatgccccttgagaggtatgtgtagaagagaagcattgccttagttgcaataacaagcctgggaagcaggagggaaggaaggaatgccgtgtagaataccgcaggcatgcggatgagctccaggtccaataacccatgaactacacactggtattttgtcagttctccatgatgattctttccctttgatacttatagggaactttacattgtaagagaaaacaaagcactgcaaaagaagtgtgccgactatcaaataaatggtgaaatcatctgcaaatgtggccaggtgagtaaattgctgtgggccagattttactttagccaacaggaatgtttaattgcatgtacatttaattttcctcttgctctctcttttgttttccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgattaatgtattcattatgctacagaactgacataagaatcaataaaatgattgttttactctgcattgaa

SEQ ID NO: 3 - RNA, 3581 nt, Homo sapiens
  source 1..3581: mol_type = mRNA
caaactctgtaagaactgcctgacagaaagctggactcaaagctcctacccgagtgtgcagcaggatcgccccggtccgggaccccaggcgcacaccgcagagtccaaagtgccgcgcctgccggccgcacctgcctgccgcggccccgcgcgccgccccgctgcccacctgcccgcctgcccacctgcccaggtgcgagtgcagccccgcgcgccggcctgagagccctgtggacaacctcgtcattgtcaggcacagagcggtagaccctgcttctctaagtgggcagcggacagcggcacgcacatttcacctgtcccgcagacaacagcaccatctgcttgggagaaccctctcccttctctgagaaagaaagatgtcgaatgggtattccacagacgagaatttccgctatctcatctcgtgcttcagggccagggtgaaaatgtacatccaggtggagcctgtgctggactacctgacctttctgcctgcagaggtgaaggagcagattcagaggacagtcgccacctccgggaacatgcaggcagttgaactgctgctgagcaccttggagaagggagtctggcaccttggttggactcgggaattcgtggaggccctccggagaaccggcagccctctggccgcccgctacatgaaccctgagctcacggacttgccctctccatcgtttgagaacgctcatgatgaatatctccaactgctgaacctccttcagcccactctggtggacaagcttctagttagagacgtcttggataagtgcatggaggaggaactgttgacaattgaagacagaaaccggattgctgctgcagaaaacaatggaaatgaatcaggtgtaagagagctactaaaaaggattgtgcagaaagaaaactggttctctgcatttctgaatgttcttcgtcaaacaggaaacaatgaacttgtccaagagttaacaggctctgattgctcagaaagcaatgcagagattgagaatttatcacaagttgatggtcctcaagtggaagagcaacttctttcaaccacagttcagccaaatctggagaaggaggtctggggcatggagaataactcatcagaatcatcttttgcagattcttctgtagtttcagaatcagacacaagtttggcagaaggaagtgtcagctgcttagatgaaagtcttggacataacagcaacatgggcagtgattcaggcaccatgggaagtgattcagatgaagagaatgtggcagcaagagcatccccggagccagaactccagctcaggccttaccaaatggaagttgcccagccagccttggaagggaagaatatcatcatctgcctccctacagggagtggaaaaaccagagtggctgtttacattgccaaggatcacttagacaagaagaaaaaagcatctgagcctggaaaagttatagttcttgtcaataaggtactgctagttgaacagctcttccgcaaggagttccaaccatttttgaagaaatggtatcgtgttattggattaagtggtgatacccaactgaaaatatcatttccagaagttgtcaagtcctgtgatattattatcagtacagctcaaatccttgaaaactccctcttaaacttggaaaatggagaagatgctggtgttcaattgtcagacttttccctcattatcattgatgaatgtcatcacaccaacaaagaagcagtgtataataacatcatgaggcattatttgatgcagaagttgaaaaacaatagactcaagaaagaaaacaaaccagtgattccccttcctcagatactgggactaacagcttcacctggtgttggaggggccacgaagcaagccaaagctgaagaacacattttaaaactatgtgccaatcttgatgcatttactattaaaactgttaaagaaaaccttgatcaactgaaaaaccaaatacaggagccatgcaagaagtttgccattgcagatgcaaccagagaagatccatttaaagagaaacttctagaaataatgacaaggattcaaacttattgtcaaatgagtccaatgtcagattttggaactcaaccctatgaacaatgggccattcaaatggaaaaaaaagctgcaaaagaaggaaatcgcaaagaacgtgtttgtgcagaacatttgaggaagtacaatgaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacagaatgaacaaaaagaagtcattagtaaatttcgcactggaaaaataaatctgcttatcgctaccacagtggcagaagaaggtctggatattaaagaatgtaacattgttatccgttatggtctcgtcaccaatgaaatagccatggtccaggcccgtggtcgagccagagctgatgagagcacctacgtcctggttgctcacagtggttcaggagttatcgaacatgagacagttaatgatttccgagagaagatgatgtataaagctatacattgtgttcaaaatatgaaaccagaggagtatgctcataagattttggaattacagatgcaaagtataatggaaaagaaaatgaaaaccaagagaaatattgccaagcattacaagaataacccatcactaataactttcctttgcaaaaactgcagtgtgctagcctgttctggggaagatatccatgtaattgagaaaatgcatcacgtcaatatgaccccagaattcaaggaactttacattgtaagagaaaacaaagcactgcaaaagaagtgtgccgactatcaaataaatggtgaaatcatctgcaaatgtggccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgattaatgtattcattatgctacagaactgacataagaatcaataaaatgattgttttactctgcattgaa

SEQ ID NO: 4 - RNA, 3234 nt, Homo sapiens
  source 1..3234: mol_type = mRNA
gcgcgccggcctgagagccctgtggacaacctcgtcattgtcaggcacagagcggtagaccctgcttctctaagtgggcagcggacagcggcacgcacatttcacctgtcccgcagacaacagcaccatctgcttgggagaaccctctcccttctctgagaaagaaagatgtcgaatgggtattccacagacgagaatttccgctatctcatctcgtgcttcagggccagggtgaaaatgtacatccaggtggagcctgtgctggactacctgacctttctgcctgcagaggtgaaggagcagattcagaggacagtcgccacctccgggaacatgcaggcagttgaactgctgctgagcaccttggagaagggagtctggcaccttggttggactcgggaattcgtggaggccctccggagaaccggcagccctctggccgcccgctacatgaaccctgagctcacggacttgccctctccatcgtttgagaacgctcatgatgaatatctccaactgctgaacctccttcagcccactctggtggacaagcttctagttagagacgtcttggataagtgcatggaggaggaactgttgacaattgaagacagaaaccggattgctgctgcagaaaacaatggaaatgaatcaggtgtaagagagctactaaaaaggattgtgcagaaagaaaactggttctctgcatttctgaatgttcttcgtcaaacaggaaacaatgaacttgtccaagagttaacaggctctgattgctcagaaagcaatgcagagattgagaatttatcacaagttgatggtcctcaagtggaagagcaacttctttcaaccacagttcagccaaatctggagaaggaggtctggggcatggagaataactcatcagaatcatcttttgcagattcttctgtagtttcagaatcagacacaagtttggcagaaggaagtgtcagctgcttagatgaaagtcttggacataacagcaacatgggcagtgattcaggcaccatgggaagtgattcagatgaagagaatgtggcagcaagagcatccccggagccagaactccagctcaggccttaccaaatggaagttgcccagccagccttggaagggaagaatatcatcatctgcctccctacagggagtggaaaaaccagagtggctgtttacattgccaaggatcacttagacaagaagaaaaaagcatctgagcctggaaaagttatagttcttgtcaataaggtactgctagttgaacagctcttccgcaaggagttccaaccatttttgaagaaatggtatcgtgttattggattaagtggtgatacccaactgaaaatatcatttccagaagttgtcaagtcctgtgatattattatcagtacagctcaaatccttgaaaactccctcttaaacttggaaaatggagaagatgctggtgttcaattgtcagacttttccctcattatcattgatgaatgtcatcacaccaacaaagaagcagtgtataataacatcatgaggcattatttgatgcagaagttgaaaaacaatagactcaagaaagaaaacaaaccagtgattccccttcctcagatactgggactaacagcttcacctggtgttggaggggccacgaagcaagccaaagctgaagaacacattttaaaagatccatttaaagagaaacttctagaaataatgacaaggattcaaacttattgtcaaatgagtccaatgtcagattttggaactcaaccctatgaacaatgggccattcaaatggaaaaaaaagctgcaaaagaaggaaatcgcaaagaacgtgtttgtgcagaacatttgaggaagtacaatgaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacagaatgaacaaaaagaagtcattagtaaatttcgcactggaaaaataaatctgcttatcgctaccacagtggcagaagaaggtctggatattaaagaatgtaacattgttatccgttatggtctcgtcaccaatgaaatagccatggtccaggcccgtggtcgagccagagctgatgagagcacctacgtcctggttgctcacagtggttcaggagttatcgaacatgagacagttaatgatttccgagagaagatgatgtataaagctatacattgtgttcaaaatatgaaaccagaggagtatgctcataagattttggaattacagatgcaaagtataatggaaaagaaaatgaaaaccaagagaaatattgccaagcattacaagaataacccatcactaataactttcctttgcaaaaactgcagtgtgctagcctgttctggggaagatatccatgtaattgagaaaatgcatcacgtcaatatgaccccagaattcaaggaactttacattgtaagagaaaacaaagcactgcaaaagaagtgtgccgactatcaaataaatggtgaaatcatctgcaaatgtggccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgattaatgtattcattatgctacagaactgacataagaatcaataaaatg

SEQ ID NO: 5 - RNA, 1776 nt, Homo sapiens
  source 1..1776: mol_type = mRNA
cacaaaccagtgattccccttcctcagatactgggactaacagcttcacctggtgttggaggggccacgaagcaagccaaagctgaagaacacattttaaaactatgtgccaatcttgatgcatttactattaaaactgttaaagaaaaccttgatcaactgaaaaaccaaatacaggagccatgcaagaagtttgccattgcagatgcaaccagagaagatccatttaaagagaaacttctagaaataatgacaaggattcaaacttattgtcaaatgagtccaatgtcagattttggaactcaaccctatgaacaatgggccattcaaatggaaaaaaaagctgcaaaagaaggaaatcgcaaagaacgtgtttgtgcagaacatttgaggaagtacaatgaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacagaatgaacaaaaagaagtcattagtaaatttcgcactggaagaataaatctgcttatcgctaccacagtggcagaagaaggtctggatattaaagaatgtaacattgttatccgttatggtctcgtcaccaatgaaatagccatggtccaggcccgtggtcgagccagagctgatgagagcacctacgtcctggttgctcacagtggttcaggagttatcgaacgtgagacagttaatgatttccgagagaagatgatgtataaagctatacattgtgttcaaaatatgaaaccagaggagtatgctcataagattttggaattacagatgcaaagtataatggaaaagaaaatgaaaaccaagagaaatattgccaagcattacaagaataacccatcactaataactttcctttgcaaaaactgcagtgtgctagcctgttctggggaagatatccatgtaattgagaaaatgcatcacgtcaatatgaccccagaattcaaggaactttacattgtaagagaaaacaaaacactgcaaaagaagtgtgccgactatcaaataaatggtgaaatcatctgcaaatgtggccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgattaatgtattcattatgctacagaactgacataagaatcaataaaatgattgttttactctgc

SEQ ID NO: 6 - RNA, 3380 nt, Homo sapiens
  source 1..3380: mol_type = mRNA
gcgcgccggcctgagagccctgtggacaacctcgtcattgtcaggcacagagcggtagaccctgcttctctaagtgggcagcggacagcggcacgcacatttcacctgtcccgcagacaacagcaccatctgcttgggagaaccctctcccttctctgagaaagaaagatgtcgaatgggtattccacagacgagaatttccgctatctcatctcgtgcttcagggccagggtgaaaatgtacatccaggtggagcctgtgctggactacctgacctttctgcctgcagaggtgaaggagcagattcagaggacagtcgccacctccgggaacatgcaggcagttgaactgctgctgagcaccttggagaagggagtctggcaccttggttggactcgggaattcgtggaggccctccggagaaccggcagccctctggccgcccgctacatgaaccctgagctcacggacttgccctctccatcgtttgagaacgctcatgatgaatatctccaactgctgaacctccttcagcccactctggtggacaagcttctagttagagacgtcttggataagtgcatggaggaggaactgttgacaattgaagacagaaaccggattgctgctgcagaaaacaatggaaatgaatcaggtgtaagagagctactaaaaaggattgtgcagaaagaaaactggttctctgcatttctgaatgttcttcgtcaaacaggaaacaatgaacttgtccaagagttaacaggctctgattgctcagaaagcaatgcagagattgagaatttatcacaagttgatggtcctcaagtggaagagcaacttctttcaaccacagttcagccaaatctggagaaggaggtctggggcatggagaataactcatcagaatcatcttttgcagattcttctgtagtttcagaatcagacacaagtttggcagaaggaagtgtcagctgcttagatgaaagtcttggacataacagcaacatgggcagtgattcaggcaccatgggaagtgattcagatgaagagaatgtggcagcaagagcatccccggagccagaactccagctcaggccttaccaaatggaagttgcccagccagccttggaagggaagaatatcatcatctgcctccctacagggagtggaaaaaccagagtggctgtttacattgccaaggatcacttagacaagaagaaaaaagcatctgagcctggaaaagttatagttcttgtcaataaggtactgctagttgaacagctcttccgcaaggagttccaaccatttttgaagaaatggtatcgtgttattggattaagtggtgatacccaactgaaaatatcatttccagaagttgtcaagtcctgtgatattattatcagtacagctcaaatccttgaaaactccctcttaaacttggaaaatggagaagatgctggtgttcaattgtcagacttttccctcattatcattgatgaatgtcatcacaccaacaaagaagcagtgtataataacatcatgaggcattatttgatgcagaagttgaaaaacaatagactcaagaaagaaaacaaaccagtgattccccttcctcagatactgggactaacagcttcacctggtgttggaggggccacgaagcaagccaaagctgaagaacacattttaaaactatgtgccaatcttgatgcatttactattaaaactgttaaagaaaaccttgatcaactgaaaaaccaaatacaggagccatgcaagaagtttgccattgcagatgcaaccagagaagatccatttaaagagaaacttctagaaataatgacaaggattcaaacttattgtcaaatgagtccaatgtcagattttggaactcaaccctatgaacaatgggccattcaaatggaaaaaaaagctgcaaaaaaaggaaatcgcaaagaacgtgtttgtgcagaacatttgaggaagtacaatgaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacagaatgaacaaaaagaagtcattagtaaatttcgcactggaaaaatcaatctgcttatcgctaccacagtggcagaagaaggtctggatattaaagaatgtaacattgttatccgttatggtctcgtcaccaatgaaatagccatggtccaggcccgtggtcgagccagagctgatgagagcacctacgtcctggttgctcacagtggttcaggagttatcgaacatgagacagttaatgatttccgagagaagatgatgtataaagctatacattgtgttcaaaatatgaaaccagaggagtatgctcataagattttggaattacagatgcaaagtataatggaaaagaaaatgaaaaccaagagaaatattgccaagcattacaagaataacccatcactaataactttcctttgcaaaaactgcagtgtgctagcctgttctggggaagatatccatgtaattgagaaaatgcatcacgtcaatatgaccccagaattcaaggaactttacattgtaagagaaaacaaagcactgcaaaagaagtgtgccgactatcaaataaatggtgaaatcatctgcaaatgtggccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgattaatgtattcattatgctacagaactgacataagaatcaataaaatgattgttttactctgaaaaaaaaaaaaaaa

SEQ ID NO: 7 - RNA, 3373 nt, Homo sapiens
  source 1..3373: mol_type = mRNA
gggccctgtggacaacctcgtcattgtcaggcacagagcggtagaccctgcttctctaagtgggcagcggacagcggcacgcacatttcacctgtcccgcagacaacagcaccatctgcttgggagaaccctctcccttctctgagaaagaaagatgtcgaatgggtattccacagacgagaatttccgctatctcatctcgtgcttcagggccagggtgaaaatgtacatccaggtggagcctgtgctggactacctgacctttctgcctgcagaggtgaaggagcagattcagaggacagtcgccacctccgggaacatgcaggcagttgaactgctgctgagcaccttggagaagggagtctggcaccttggttggactcgggaattcgtggaggccctccggagaaccggcagccctctggccgcccgctacatgaaccctgagctcacggacttgccctctccatcgtttgagaacgctcatgatgaatatctccaactgctgaacctccttcagcccactctggtggacaagcttctagttagagacgtcttggataagtgcatggaggaggaactgttgacaattgaagacagaaaccggattgctgctgcagaaaacaatggaaatgaatcaggtgtaagagagctactaaaaaggattgtgcagaaagaaaactggttctctgcatttctgaatgttcttcgtcaaacaggaaacaatgaacttgtccaagagttaacaggctctgattgctcagaaagcaatgcagagattgagaatttatcacaagttgatggtcctcaagtggaagagcaacttctttcaaccacagttcagccaaatctggagaaggaggtctggggcatggagaataactcatcagaatcatcttttgcagattcttctgtagtttcagaatcagacacaagtttggcagaaggaagtgtcagctgcttagatgaaagtcttggacataacagcaacatgggcagtgattcaggcaccatgggaagtgattcagatgaagagaatgtggcagcaagagcatccccggagccagaactccagctcaggccttaccaaatggaagttgcccagccagccttggaagggaagaatatcatcatctgcctccctacagggagtggaaaaaccagagtggctgtttacattgccaaggatcacttagacaagaagaaaaaagcatctgagcctggaaaagttatagttcttgtcaataaggtactgctagttgaacagctcttccgcaaggagttccaaccatttttgaagaaatggtatcgtgttattggattaagtggtgatacccaactgaaaatatcatttccagaagttgtcaagtcctgtgatattattatcagtacagctcaaatccttgaaaactccctcttaaacttggaaaatggagaagatgctggtgttcaattgtcagacttttccttcattatcattgatgaatgtcatcacaccaacaaagaagcagtgtataataacatcatgaggcattatttgatgcagaagttgaaaaacaatagactcaagaaagaaaacaaaccagtgattccccttcctcagatactgggactaacagcttcacctggtgttggaggggccacgaagcaagccaaagctgaagaacacattttaaaactatgtgccaatcttgatgcatttactattaaaactgttaaagaaaaccttgatcaactgaaaaaccaaatacaggagccatgcaagaagtttgccattgcagatgcaaccagagaagatccatttaaagagaaacttctagaaataatgacaaggattcaaacttattgtcaaatgagtccaatgtcagattttggaactcaaccctatgaacaatgggccattcaaatggaaaaaaaagctgcaaaagaaggaaatcgcaaagaaagtgtttgtgcagaacatttgaggaagtacaataaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacagaatgaacaaaaagaagtcattagtaaatttcgcactggaaaaataaatctgcttatcgctaccacagtggcagaagaaggtctggatattaaagaatgtaacattgttatccgttatggtctcgtcaccaatgaaatagccatggtccaggcccgtggtcgagccagagctgatgagagcacctacgtcctggttgctcacagtggttcaggagttatcgaacgtgagacagttaatgatttccgagagaagatgatgtataaagctatacattgtgttcaaaatatgaaaccagaggagtatgctcataagattttggaattacagatgcaaagtataatggaaaagaaaatgaaaaccaagagaaatattgccaagcattacaagaataacccatcactaataactttcctttgcaaaaactgcagtgtgctagcctgttctggggaagatatccatgtaattgagaaaatgcatcacgtcaatatgaccccagaattcaaggaactttacattgtaagagaaaacaaagcactgcaaaagaagtgtgccgactatcaaataaatggtgaaatcatctgcaaatgtggccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgattaatgtattcattatgctacagaactgacataagaatcaataaaatgattgttttactctccaaaaaaaaaaaaaaaaaaaaa

SEQ ID NO: 8 - RNA, 3244 nt, Homo sapiens
  source 1..3244: mol_type = mRNA
ttcacctgtcccgcagacaacagcaccatctgcttgggagaaccctctcccttctctgagaaagaaagatgtcgaatgggtattccacagacgagaatttccgctatctcatctcgtgcttcagggccagggtgaaaatgtacatccaggtggagcctgtgctggactacctgacctttctgcctgcagaggtgaaggagcagattcagaggacagtcgccacctccgggaacatgcaggcagttgaactgctgctgagcaccttggagaagggagtctggcaccttggttggactcgggaattcgtggaggccctccggagaaccggcagccctctggccgcccgctacatgaaccctgagctcacggacttgccctctccatcgtttgagaacgctcatgatgaatatctccaactgctgaacctccttcagcccactctggtggacaagcttctagttagagacgtcttggataagtgcatggaggaggaactgttgacaattgaagacagaaaccggattgctgctgcagaaaacaatggaaatgaatcaggtgtaagagagctactaaaaaggattgtgcagaaagaaaactggttctctgcatttctgaatgttcttcgtcaaacaggaaacaatgaacttgtccaagagttaacaggctctgattgctcagaaagcaatgcagagattgagaatttatcacaagttgatggtcctcaagtggaagagcaacttctttcaaccacagttcagccaaatctggagaaggaggtctggggcatggagaataactcatcagaatcatcttttgcagattcttctgtagtttcagaatcagacacaagtttggcagaaggaagtgtcagctgcttagatgaaagtcttggacataacagcaacatgggcagtgattcaggcaccatgggaagtgattcagatgaagagaatgtggcagcaagagcatccccggagccagaactccagctcaggccttaccaaatggaagttgcccagccagccttggaagggaagaatatcatcatctgcctccctacagggagtggaaaaaccagagtggctgtttacattgccaaggatcacttagacaagaagaaaaaagcatctgagcctggaaaagttatagttcttgtcaataaggtactgctagttgaacagctcttccgcaaggagttccaaccatttttgaagaaatggtatcgtgttattggattaagtggtgatacccaactgaaaatatcatttccagaagttgtcaagtcctgtgatattattatcagtacagctcaaatccttgaaaactccctcttaaacttggaaaatggagaagatgctggtgttcaattgtcagacttttccctcattatcattgatgaatgtcatcacaccaacaaagaagcagtgtataataacatcatgaggcattatttgatgcagaagttgaaaaacaatagactcaagaaagaaaacaaaccagtgattccccttcctcagatactgggactaacagcttcacctggtgttggaggggccacgaagcaagccaaagctgaagaacacattttaaaactatgtgccaatcttgatgcatttactattaaaactgttaaagaaaaccttgatcaactgaaaaaccaaatacaggagccatgcaagaagtttgccattgcagatgcaaccagagaagatccatttaaagagaaacttctagaaataatgacaaggattcaaacttattgtcaaatgagtccaatgtcagattttggaactcaaccctatgaacaatgggccattcaaatggaaaaaaaagctgcaaaagaaggaaatcgcaaagaacgtgtttgtgcagaacatttgaggaagtacaatgaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacagaatgaacaaaaagaagtcattagtaaatttcgcactggaaaaataaatctgcttatcgctaccacagtggcagaagaaggtctggatattaaagaatgtaacattgttatccgttatggtctcgtcaccaatgaaatagccatggtccaggcccgtggtcgagccagagctgatgagagcacctacgtcctggttgctcacagtggttcaggagttatcgaacgtgagacagttaatgatttccgagagaagatgatgtataaagctatacattgtgttcaaaatatgaaaccagaggagtatgctcataagattttggaattacagatgcaaagtataatggaaaagaaaatgaaaaccaagagaaatattgccaagcattacaagaataacccatcactaataactttcctttgcaaaaactgcagtgtgctagcctgttctggggaagatatccatgtaattgagaaaatgcatcacgtcaatatgaccccagaattcaaggaactttacattgtaagagaaaacaaaacactgcaaaagaagtgtgccgactatcaaataaatggtgaaatcatctgcaaatgtggccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgattaatgtattcattatgctacagaactgacataagaatcaa

SEQ ID NO: 9 - RNA, 3452 nt, Homo sapiens
  source 1..3452: mol_type = mRNA
acagaaaccaaagtcaggcaaactctgtaagaactgcctgacagaaagctggactcaaagctcctacccgagtgtgcagcaggatcgccccggtccgggaccccaggcgcacaccgcagagtccaaagtgccgcgcctgccggccgcacccgccggcctgagagccctgtggacaacctcgtcattgtcaggcacagagcggtagaccctgcttctctaagtgggcagcggacagcggcacgcacatttcacctgtcccgcagacaacagcaccatctgcttgggagaaccctctcccttctctgagaaagaaagatgtcgaatgggtattccacagacgagaatttccgctatctcatctcgtgcttcagggccagggtgaaaatgtacatccaggtggagcctgtgctggactacctgacctttctgcctgcagaggtgaaggagcagattcagaggacagtcgccacctccgggaacatgcaggcagttgaactgctgctgagcaccttggagaagggagtctggcaccttggttggactcgggaattcgtggaggccctccggagaaccggcagccctctggccgcccgctacatgaaccctgagctcacggacttgccctctccatcgtttgagaacgctcatgatgaatatctccaactgctgaacctccttcagcccactctggtggacaagcttctagttagagacgtcttggataagtgcatggaggaggaactgttgacaattgaagacagaaaccggattgctgctgcagaaaacaatggaaatgaatcaggtgtaagagagctactaaaaaggattgtgcagaaagaaaactggttctctgcatttctgaatgttcttcgtcaaacaggaaacaatgaacttgtccaagagttaacaggctctgattgctcagaaagcaatgcagagattgagaatttatcacaagttgatggtcctcaagtggaagagcaacttctttcaaccacagttcagccaaatctggagaaggaggtctggggcatggagaataactcatcagaatcatcttttgcagattcttctgtagtttcagaatcagacacaagtttggcagaaggaagtgtcagctgcttagatgaaagtcttggacataacagcaacatgggcagtgattcaggcaccgtgggaagtgattcagatgaagagaatgtggcagcaagagcatccccggagccagaactccagctcaggccttaccaaatggaagttgcccagccagccttggaagggaagaatatcatcatctgcctccctacagggagtggaaaaaccagagtggctgtttacattgccaaggatcacttagacaagaagaaaaaagcatctgagcctggaaaagttatagttcttgtcaataaggtactgctagttgaacagctcttccgcaaggagttccaaccatttttgaagaaatggtatcgtgttattggattaagtggtgatacccaactgaaaatatcatttccagaagttgtcaagtcctgtgatattattatcagtacagctcaaatccttgaaaactccctcttaaacttggaaaatggagaagatgctggtgttcaattgtcagacttttccctcattatcattgatgaatgtcatcacaccaacaaagaagcagtgtataataacatcatgaggcattatttgatgcagaagttgaaaaacaatagactcaagaaagaaaacaaaccagtgattccccttcctcagatactgggactaacagcttcacctggtgttggaggggccacgaagcaagccaaagctgaagaacacattttaaaactatgtgccaatcttgatgcatttactattaaaactgttaaagaaaaccttgatcaactgaaaaaccaaatacaggagccatgcaagaagtttgccattgcagatgcaaccagagaagatccatttaaagagaaacttctagaaataatgacaaggattcaaacttattgtcaaatgagtccaatgtcagattttggaactcaaccctatgaacaatgggccattcaaatggaaaaaaaagctgcaaaagaaggaaatcgcaaagaacgtgtttgtgcagaacatttgaggaagtacaatgaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacagaatgaacaaaaagaagtcattagtaaatttcgcactggaaaaataaatctgcttatcgctaccacagtggcagaagaaggtctggatattaaagaatgtaacattgttatccgttatggtctcgtcaccaatgaaatagccatggtccaggcccgtggtcgagccagagctgatgagagcacctgcgtcctggttgctcacagtggttcaggagttatcgaacgtgagacagttaatgatttccgagagaagatgatgtataaagctatacattgtgttcaaaatatgaaaccagaggagtatgctcataagattttggaattacagatgcaaagtataatggaaaagaaaatgaaaaccaagagaaatattgccaagcatttcaagaataacccatcactaataactttcctttgcaaaaactgcagtgtgctagcctgttctggggaagatatccatgtaattgagaaaatgcatcacgtcaatatgaccccagaattcaaggaactttacattgtaagagaaaacaaaacactgcaaaagaagtgtgccgactatcaaataaatggtgaaatcatctgcaaatgtggccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgatt

SEQ ID NO: 10 - RNA, 2540 nt, Homo sapiens
  source 1..2540: mol_type = mRNA
cgccccgctgcccacctgcccgcctgcccacctgcccaggtgcgagtgcagccccgcgcgccggcctgagagccctgtggacaacctcgtcattgtcaggcacagagcggtagaccctgcttctctaagtgggcagcggacagcggcacgcacatttcacctgtcccgcagacaacagcaccatctgcttgggagaaccctctcccttctctgagaaagaaagatgtcgaatgggtattccacagacgagaatttccgctatctcatctcgtgcttcagggccagggtgaaaatgtacatccaggtggagcctgtgctggactacctgacctttctgcctgcagaggtgaaggagcagattcagaggacagtcgccacctccgggaacatgcaggcagttgaactgctgctgagcaccttggagaagggagtctggcaccttggttggactcgggaattcgtggaggccctccggagaaccggcagccctctggccgcccgctacatgaaccctgagctcacggacttgccctctccatcgtttgagaacgctcatgatgaatatctccaactgctgaacctccttcagcccactctggtggacaagcttctagttagagacgtcttggataagtgcatggaggaggaactgttgacaattgaagacagaaaccggattgctgctgcagaaaacaatggaaatgaatcaggtgtaagagagctactaaaaaggattgtgcagaaagaaaactggttctctgcatttctgaatgttcttcgtcaaacaggaaacaatgaacttgtccaagagttaacaggctctgattgctcagaaagcaatgcagagattgagaatttatcacaagttgatggtcctcaagtggaagagcaacttctttcaaccacagttcagccaaatctggagaaggaggtctggggcatggagaataactcatcagaatcatcttttgcagattcttctgtagtttcagaatcagacacaagtttggcagaaggaagtgtcagctgcttagatgaaagtcttggacataacagcaacatgggcagtgattcaggcaccatgggaagtgattcagatgaagagaatgtggcagcaagagcatccccggagccagaactccagctcaggccttaccaaatggaagttgcccagccagccttggaagggaagaatatcatcatctgcctccctacagggagtggaaaaaccagagtggctgtttacattgccaaggatcacttagacaagaagaaaaaagcatctgagcctggaaaagttatagttcttgtcaataaggtactgctagttgaacagctcttccgcaaggagttccaaccatttttgaagaaatggtatcgtgttattggattaagtggtgatacccaactgaaaatatcatttccagaagttgtcaagtcctgtgatattattatcagtacagctcaaatccttgaaaactccctcttaaacttggaaaatggagaagatgctggtgttcaattgtcagacttttccctcattatcattgatgaatgtcatcacaccaacaaagaagcagtgtataataacatcatgaggcattatttgatgcagaagttgaaaaacaatagactcaagaaagaaaacaaaccagtgattccccttcctcagatactgggactaacagcttcacctggtgttggaggggccacgaagcaagccaaagctgaagaacacattttaaaactatgtgccaatcttgatgcatttactattaaaactgttaaagaaaaccttgatcaactgaaaaaccaaatacaggagccatgcaagaagtttgccattgcagatgcaaccagagaagatccatttaaagagaaacttctagaaataatgacaaggattcaaacttattgtcaaatgagtccaatgtcagattttggaactcaaccctatgaacaatgggccattcaaatggaaaaaaaagctgcaaaagaaggaaatcgcaaagaacgtgtttgtgcagaacatttgaggaagtacaatgaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacaaaaaaaaaaaaaaa

SEQ ID NO: 11 - DNA, 3581 bp, Homo sapiens
  source 1..3581: mol_type = other DNA
caaactctgtaagaactgcctgacagaaagctggactcaaagctcctacccgagtgtgcagcaggatcgccccggtccgggaccccaggcgcacaccgcagagtccaaagtgccgcgcctgccggccgcacctgcctgccgcggccccgcgcgccgccccgctgcccacctgcccgcctgcccacctgcccaggtgcgagtgcagccccgcgcgccggcctgagagccctgtggacaacctcgtcattgtcaggcacagagcggtagaccctgcttctctaagtgggcagcggacagcggcacgcacatttcacctgtcccgcagacaacagcaccatctgcttgggagaaccctctcccttctctgagaaagaaagatgtcgaatgggtattccacagacgagaatttccgctatctcatctcgtgcttcagggccagggtgaaaatgtacatccaggtggagcctgtgctggactacctgacctttctgcctgcagaggtgaaggagcagattcagaggacagtcgccacctccgggaacatgcaggcagttgaactgctgctgagcaccttggagaagggagtctggcaccttggttggactcgggaattcgtggaggccctccggagaaccggcagccctctggccgcccgctacatgaaccctgagctcacggacttgccctctccatcgtttgagaacgctcatgatgaatatctccaactgctgaacctccttcagcccactctggtggacaagcttctagttagagacgtcttggataagtgcatggaggaggaactgttgacaattgaagacagaaaccggattgctgctgcagaaaacaatggaaatgaatcaggtgtaagagagctactaaaaaggattgtgcagaaagaaaactggttctctgcatttctgaatgttcttcgtcaaacaggaaacaatgaacttgtccaagagttaacaggctctgattgctcagaaagcaatgcagagattgagaatttatcacaagttgatggtcctcaagtggaagagcaacttctttcaaccacagttcagccaaatctggagaaggaggtctggggcatggagaataactcatcagaatcatcttttgcagattcttctgtagtttcagaatcagacacaagtttggcagaaggaagtgtcagctgcttagatgaaagtcttggacataacagcaacatgggcagtgattcaggcaccatgggaagtgattcagatgaagagaatgtggcagcaagagcatccccggagccagaactccagctcaggccttaccaaatggaagttgcccagccagccttggaagggaagaatatcatcatctgcctccctacagggagtggaaaaaccagagtggctgtttacattgccaaggatcacttagacaagaagaaaaaagcatctgagcctggaaaagttatagttcttgtcaataaggtactgctagttgaacagctcttccgcaaggagttccaaccatttttgaagaaatggtatcgtgttattggattaagtggtgatacccaactgaaaatatcatttccagaagttgtcaagtcctgtgatattattatcagtacagctcaaatccttgaaaactccctcttaaacttggaaaatggagaagatgctggtgttcaattgtcagacttttccctcattatcattgatgaatgtcatcacaccaacaaagaagcagtgtataataacatcatgaggcattatttgatgcagaagttgaaaaacaatagactcaagaaagaaaacaaaccagtgattccccttcctcagatactgggactaacagcttcacctggtgttggaggggccacgaagcaagccaaagctgaagaacacattttaaaactatgtgccaatcttgatgcatttactattaaaactgttaaagaaaaccttgatcaactgaaaaaccaaatacaggagccatgcaagaagtttgccattgcagatgcaaccagagaagatccatttaaagagaaacttctagaaataatgacaaggattcaaacttattgtcaaatgagtccaatgtcagattttggaactcaaccctatgaacaatgggccattcaaatggaaaaaaaagctgcaaaagaaggaaatcgcaaagaacgtgtttgtgcagaacatttgaggaagtacaatgaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacagaatgaacaaaaagaagtcattagtaaatttcgcactggaaaaataaatctgcttatcgctaccacagtggcagaagaaggtctggatattaaagaatgtaacattgttatccgttatggtctcgtcaccaatgaaatagccatggtccaggcccgtggtcgagccagagctgatgagagcacctacgtcctggttgctcacagtggttcaggagttatcgaacatgagacagttaatgatttccgagagaagatgatgtataaagctatacattgtgttcaaaatatgaaaccagaggagtatgctcataagattttggaattacagatgcaaagtataatggaaaagaaaatgaaaaccaagagaaatattgccaagcattacaagaataacccatcactaataactttcctttgcaaaaactgcagtgtgctagcctgttctggggaagatatccatgtaattgagaaaatgcatcacgtcaatatgaccccagaattcaaggaactttacattgtaagagaaaacaaagcactgcaaaagaagtgtgccgactatcaaataaatggtgaaatcatctgcaaatgtggccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgattaatgtattcattatgctacagaactgacataagaatcaataaaatgattgttttactctgcattgaa

SEQ ID NO: 12 - DNA, 3234 bp, Homo sapiens
  source 1..3234: mol_type = other DNA
gcgcgccggcctgagagccctgtggacaacctcgtcattgtcaggcacagagcggtagaccctgcttctctaagtgggcagcggacagcggcacgcacatttcacctgtcccgcagacaacagcaccatctgcttgggagaaccctctcccttctctgagaaagaaagatgtcgaatgggtattccacagacgagaatttccgctatctcatctcgtgcttcagggccagggtgaaaatgtacatccaggtggagcctgtgctggactacctgacctttctgcctgcagaggtgaaggagcagattcagaggacagtcgccacctccgggaacatgcaggcagttgaactgctgctgagcaccttggagaagggagtctggcaccttggttggactcgggaattcgtggaggccctccggagaaccggcagccctctggccgcccgctacatgaaccctgagctcacggacttgccctctccatcgtttgagaacgctcatgatgaatatctccaactgctgaacctccttcagcccactctggtggacaagcttctagttagagacgtcttggataagtgcatggaggaggaactgttgacaattgaagacagaaaccggattgctgctgcagaaaacaatggaaatgaatcaggtgtaagagagctactaaaaaggattgtgcagaaagaaaactggttctctgcatttctgaatgttcttcgtcaaacaggaaacaatgaacttgtccaagagttaacaggctctgattgctcagaaagcaatgcagagattgagaatttatcacaagttgatggtcctcaagtggaagagcaacttctttcaaccacagttcagccaaatctggagaaggaggtctggggcatggagaataactcatcagaatcatcttttgcagattcttctgtagtttcagaatcagacacaagtttggcagaaggaagtgtcagctgcttagatgaaagtcttggacataacagcaacatgggcagtgattcaggcaccatgggaagtgattcagatgaagagaatgtggcagcaagagcatccccggagccagaactccagctcaggccttaccaaatggaagttgcccagccagccttggaagggaagaatatcatcatctgcctccctacagggagtggaaaaaccagagtggctgtttacattgccaaggatcacttagacaagaagaaaaaagcatctgagcctggaaaagttatagttcttgtcaataaggtactgctagttgaacagctcttccgcaaggagttccaaccatttttgaagaaatggtatcgtgttattggattaagtggtgatacccaactgaaaatatcatttccagaagttgtcaagtcctgtgatattattatcagtacagctcaaatccttgaaaactccctcttaaacttggaaaatggagaagatgctggtgttcaattgtcagacttttccctcattatcattgatgaatgtcatcacaccaacaaagaagcagtgtataataacatcatgaggcattatttgatgcagaagttgaaaaacaatagactcaagaaagaaaacaaaccagtgattccccttcctcagatactgggactaacagcttcacctggtgttggaggggccacgaagcaagccaaagctgaagaacacattttaaaagatccatttaaagagaaacttctagaaataatgacaaggattcaaacttattgtcaaatgagtccaatgtcagattttggaactcaaccctatgaacaatgggccattcaaatggaaaaaaaagctgcaaaagaaggaaatcgcaaagaacgtgtttgtgcagaacatttgaggaagtacaatgaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacagaatgaacaaaaagaagtcattagtaaatttcgcactggaaaaataaatctgcttatcgctaccacagtggcagaagaaggtctggatattaaagaatgtaacattgttatccgttatggtctcgtcaccaatgaaatagccatggtccaggcccgtggtcgagccagagctgatgagagcacctacgtcctggttgctcacagtggttcaggagttatcgaacatgagacagttaatgatttccgagagaagatgatgtataaagctatacattgtgttcaaaatatgaaaccagaggagtatgctcataagattttggaattacagatgcaaagtataatggaaaagaaaatgaaaaccaagagaaatattgccaagcattacaagaataacccatcactaataactttcctttgcaaaaactgcagtgtgctagcctgttctggggaagatatccatgtaattgagaaaatgcatcacgtcaatatgaccccagaattcaaggaactttacattgtaagagaaaacaaagcactgcaaaagaagtgtgccgactatcaaataaatggtgaaatcatctgcaaatgtggccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgattaatgtattcattatgctacagaactgacataagaatcaataaaatg

SEQ ID NO: 13 - DNA, 1776 bp, Homo sapiens
  source 1..1776: mol_type = other DNA
cacaaaccagtgattccccttcctcagatactgggactaacagcttcacctggtgttggaggggccacgaagcaagccaaagctgaagaacacattttaaaactatgtgccaatcttgatgcatttactattaaaactgttaaagaaaaccttgatcaactgaaaaaccaaatacaggagccatgcaagaagtttgccattgcagatgcaaccagagaagatccatttaaagagaaacttctagaaataatgacaaggattcaaacttattgtcaaatgagtccaatgtcagattttggaactcaaccctatgaacaatgggccattcaaatggaaaaaaaagctgcaaaagaaggaaatcgcaaagaacgtgtttgtgcagaacatttgaggaagtacaatgaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacagaatgaacaaaaagaagtcattagtaaatttcgcactggaagaataaatctgcttatcgctaccacagtggcagaagaaggtctggatattaaagaatgtaacattgttatccgttatggtctcgtcaccaatgaaatagccatggtccaggcccgtggtcgagccagagctgatgagagcacctacgtcctggttgctcacagtggttcaggagttatcgaacgtgagacagttaatgatttccgagagaagatgatgtataaagctatacattgtgttcaaaatatgaaaccagaggagtatgctcataagattttggaattacagatgcaaagtataatggaaaagaaaatgaaaaccaagagaaatattgccaagcattacaagaataacccatcactaataactttcctttgcaaaaactgcagtgtgctagcctgttctggggaagatatccatgtaattgagaaaatgcatcacgtcaatatgaccccagaattcaaggaactttacattgtaagagaaaacaaaacactgcaaaagaagtgtgccgactatcaaataaatggtgaaatcatctgcaaatgtggccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgattaatgtattcattatgctacagaactgacataagaatcaataaaatgattgttttactctgc

SEQ ID NO: 14 - DNA, 3380 bp, Homo sapiens
  source 1..3380: mol_type = other DNA
gcgcgccggcctgagagccctgtggacaacctcgtcattgtcaggcacagagcggtagaccctgcttctctaagtgggcagcggacagcggcacgcacatttcacctgtcccgcagacaacagcaccatctgcttgggagaaccctctcccttctctgagaaagaaagatgtcgaatgggtattccacagacgagaatttccgctatctcatctcgtgcttcagggccagggtgaaaatgtacatccaggtggagcctgtgctggactacctgacctttctgcctgcagaggtgaaggagcagattcagaggacagtcgccacctccgggaacatgcaggcagttgaactgctgctgagcaccttggagaagggagtctggcaccttggttggactcgggaattcgtggaggccctccggagaaccggcagccctctggccgcccgctacatgaaccctgagctcacggacttgccctctccatcgtttgagaacgctcatgatgaatatctccaactgctgaacctccttcagcccactctggtggacaagcttctagttagagacgtcttggataagtgcatggaggaggaactgttgacaattgaagacagaaaccggattgctgctgcagaaaacaatggaaatgaatcaggtgtaagagagctactaaaaaggattgtgcagaaagaaaactggttctctgcatttctgaatgttcttcgtcaaacaggaaacaatgaacttgtccaagagttaacaggctctgattgctcagaaagcaatgcagagattgagaatttatcacaagttgatggtcctcaagtggaagagcaacttctttcaaccacagttcagccaaatctggagaaggaggtctggggcatggagaataactcatcagaatcatcttttgcagattcttctgtagtttcagaatcagacacaagtttggcagaaggaagtgtcagctgcttagatgaaagtcttggacataacagcaacatgggcagtgattcaggcaccatgggaagtgattcagatgaagagaatgtggcagcaagagcatccccggagccagaactccagctcaggccttaccaaatggaagttgcccagccagccttggaagggaagaatatcatcatctgcctccctacagggagtggaaaaaccagagtggctgtttacattgccaaggatcacttagacaagaagaaaaaagcatctgagcctggaaaagttatagttcttgtcaataaggtactgctagttgaacagctcttccgcaaggagttccaaccatttttgaagaaatggtatcgtgttattggattaagtggtgatacccaactgaaaatatcatttccagaagttgtcaagtcctgtgatattattatcagtacagctcaaatccttgaaaactccctcttaaacttggaaaatggagaagatgctggtgttcaattgtcagacttttccctcattatcattgatgaatgtcatcacaccaacaaagaagcagtgtataataacatcatgaggcattatttgatgcagaagttgaaaaacaatagactcaagaaagaaaacaaaccagtgattccccttcctcagatactgggactaacagcttcacctggtgttggaggggccacgaagcaagccaaagctgaagaacacattttaaaactatgtgccaatcttgatgcatttactattaaaactgttaaagaaaaccttgatcaactgaaaaaccaaatacaggagccatgcaagaagtttgccattgcagatgcaaccagagaagatccatttaaagagaaacttctagaaataatgacaaggattcaaacttattgtcaaatgagtccaatgtcagattttggaactcaaccctatgaacaatgggccattcaaatggaaaaaaaagctgcaaaaaaaggaaatcgcaaagaacgtgtttgtgcagaacatttgaggaagtacaatgaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacagaatgaacaaaaagaagtcattagtaaatttcgcactggaaaaatcaatctgcttatcgctaccacagtggcagaagaaggtctggatattaaagaatgtaacattgttatccgttatggtctcgtcaccaatgaaatagccatggtccaggcccgtggtcgagccagagctgatgagagcacctacgtcctggttgctcacagtggttcaggagttatcgaacatgagacagttaatgatttccgagagaagatgatgtataaagctatacattgtgttcaaaatatgaaaccagaggagtatgctcataagattttggaattacagatgcaaagtataatggaaaagaaaatgaaaaccaagagaaatattgccaagcattacaagaataacccatcactaataactttcctttgcaaaaactgcagtgtgctagcctgttctggggaagatatccatgtaattgagaaaatgcatcacgtcaatatgaccccagaattcaaggaactttacattgtaagagaaaacaaagcactgcaaaagaagtgtgccgactatcaaataaatggtgaaatcatctgcaaatgtggccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgattaatgtattcattatgctacagaactgacataagaatcaataaaatgattgttttactctgaaaaaaaaaaaaaaa

SEQ ID NO: 15 - DNA, 3373 bp, Homo sapiens
  source 1..3373: mol_type = other DNA
gggccctgtggacaacctcgtcattgtcaggcacagagcggtagaccctgcttctctaagtgggcagcggacagcggcacgcacatttcacctgtcccgcagacaacagcaccatctgcttgggagaaccctctcccttctctgagaaagaaagatgtcgaatgggtattccacagacgagaatttccgctatctcatctcgtgcttcagggccagggtgaaaatgtacatccaggtggagcctgtgctggactacctgacctttctgcctgcagaggtgaaggagcagattcagaggacagtcgccacctccgggaacatgcaggcagttgaactgctgctgagcaccttggagaagggagtctggcaccttggttggactcgggaattcgtggaggccctccggagaaccggcagccctctggccgcccgctacatgaaccctgagctcacggacttgccctctccatcgtttgagaacgctcatgatgaatatctccaactgctgaacctccttcagcccactctggtggacaagcttctagttagagacgtcttggataagtgcatggaggaggaactgttgacaattgaagacagaaaccggattgctgctgcagaaaacaatggaaatgaatcaggtgtaagagagctactaaaaaggattgtgcagaaagaaaactggttctctgcatttctgaatgttcttcgtcaaacaggaaacaatgaacttgtccaagagttaacaggctctgattgctcagaaagcaatgcagagattgagaatttatcacaagttgatggtcctcaagtggaagagcaacttctttcaaccacagttcagccaaatctggagaaggaggtctggggcatggagaataactcatcagaatcatcttttgcagattcttctgtagtttcagaatcagacacaagtttggcagaaggaagtgtcagctgcttagatgaaagtcttggacataacagcaacatgggcagtgattcaggcaccatgggaagtgattcagatgaagagaatgtggcagcaagagcatccccggagccagaactccagctcaggccttaccaaatggaagttgcccagccagccttggaagggaagaatatcatcatctgcctccctacagggagtggaaaaaccagagtggctgtttacattgccaaggatcacttagacaagaagaaaaaagcatctgagcctggaaaagttatagttcttgtcaataaggtactgctagttgaacagctcttccgcaaggagttccaaccatttttgaagaaatggtatcgtgttattggattaagtggtgatacccaactgaaaatatcatttccagaagttgtcaagtcctgtgatattattatcagtacagctcaaatccttgaaaactccctcttaaacttggaaaatggagaagatgctggtgttcaattgtcagacttttccttcattatcattgatgaatgtcatcacaccaacaaagaagcagtgtataataacatcatgaggcattatttgatgcagaagttgaaaaacaatagactcaagaaagaaaacaaaccagtgattccccttcctcagatactgggactaacagcttcacctggtgttggaggggccacgaagcaagccaaagctgaagaacacattttaaaactatgtgccaatcttgatgcatttactattaaaactgttaaagaaaaccttgatcaactgaaaaaccaaatacaggagccatgcaagaagtttgccattgcagatgcaaccagagaagatccatttaaagagaaacttctagaaataatgacaaggattcaaacttattgtcaaatgagtccaatgtcagattttggaactcaaccctatgaacaatgggccattcaaatggaaaaaaaagctgcaaaagaaggaaatcgcaaagaaagtgtttgtgcagaacatttgaggaagtacaataaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacagaatgaacaaaaagaagtcattagtaaatttcgcactggaaaaataaatctgcttatcgctaccacagtggcagaagaaggtctggatattaaagaatgtaacattgttatccgttatggtctcgtcaccaatgaaatagccatggtccaggcccgtggtcgagccagagctgatgagagcacctacgtcctggttgctcacagtggttcaggagttatcgaacgtgagacagttaatgatttccgagagaagatgatgtataaagctatacattgtgttcaaaatatgaaaccagaggagtatgctcataagattttggaattacagatgcaaagtataatggaaaagaaaatgaaaaccaagagaaatattgccaagcattacaagaataacccatcactaataactttcctttgcaaaaactgcagtgtgctagcctgttctggggaagatatccatgtaattgagaaaatgcatcacgtcaatatgaccccagaattcaaggaactttacattgtaagagaaaacaaagcactgcaaaagaagtgtgccgactatcaaataaatggtgaaatcatctgcaaatgtggccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgattaatgtattcattatgctacagaactgacataagaatcaataaaatgattgttttactctccaaaaaaaaaaaaaaaaaaaaa

SEQ ID NO: 16 - DNA, 3244 bp, Homo sapiens
  source 1..3244: mol_type = other DNA
ttcacctgtcccgcagacaacagcaccatctgcttgggagaaccctctcccttctctgagaaagaaagatgtcgaatgggtattccacagacgagaatttccgctatctcatctcgtgcttcagggccagggtgaaaatgtacatccaggtggagcctgtgctggactacctgacctttctgcctgcagaggtgaaggagcagattcagaggacagtcgccacctccgggaacatgcaggcagttgaactgctgctgagcaccttggagaagggagtctggcaccttggttggactcgggaattcgtggaggccctccggagaaccggcagccctctggccgcccgctacatgaaccctgagctcacggacttgccctctccatcgtttgagaacgctcatgatgaatatctccaactgctgaacctccttcagcccactctggtggacaagcttctagttagagacgtcttggataagtgcatggaggaggaactgttgacaattgaagacagaaaccggattgctgctgcagaaaacaatggaaatgaatcaggtgtaagagagctactaaaaaggattgtgcagaaagaaaactggttctctgcatttctgaatgttcttcgtcaaacaggaaacaatgaacttgtccaagagttaacaggctctgattgctcagaaagcaatgcagagattgagaatttatcacaagttgatggtcctcaagtggaagagcaacttctttcaaccacagttcagccaaatctggagaaggaggtctggggcatggagaataactcatcagaatcatcttttgcagattcttctgtagtttcagaatcagacacaagtttggcagaaggaagtgtcagctgcttagatgaaagtcttggacataacagcaacatgggcagtgattcaggcaccatgggaagtgattcagatgaagagaatgtggcagcaagagcatccccggagccagaactccagctcaggccttaccaaatggaagttgcccagccagccttggaagggaagaatatcatcatctgcctccctacagggagtggaaaaaccagagtggctgtttacattgccaaggatcacttagacaagaagaaaaaagcatctgagcctggaaaagttatagttcttgtcaataaggtactgctagttgaacagctcttccgcaaggagttccaaccatttttgaagaaatggtatcgtgttattggattaagtggtgatacccaactgaaaatatcatttccagaagttgtcaagtcctgtgatattattatcagtacagctcaaatccttgaaaactccctcttaaacttggaaaatggagaagatgctggtgttcaattgtcagacttttccctcattatcattgatgaatgtcatcacaccaacaaagaagcagtgtataataacatcatgaggcattatttgatgcagaagttgaaaaacaatagactcaagaaagaaaacaaaccagtgattccccttcctcagatactgggactaacagcttcacctggtgttggaggggccacgaagcaagccaaagctgaagaacacattttaaaactatgtgccaatcttgatgcatttactattaaaactgttaaagaaaaccttgatcaactgaaaaaccaaatacaggagccatgcaagaagtttgccattgcagatgcaaccagagaagatccatttaaagagaaacttctagaaataatgacaaggattcaaacttattgtcaaatgagtccaatgtcagattttggaactcaaccctatgaacaatgggccattcaaatggaaaaaaaagctgcaaaagaaggaaatcgcaaagaacgtgtttgtgcagaacatttgaggaagtacaatgaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacagaatgaacaaaaagaagtcattagtaaatttcgcactggaaaaataaatctgcttatcgctaccacagtggcagaagaaggtctggatattaaagaatgtaacattgttatccgttatggtctcgtcaccaatgaaatagccatggtccaggcccgtggtcgagccagagctgatgagagcacctacgtcctggttgctcacagtggttcaggagttatcgaacgtgagacagttaatgatttccgagagaagatgatgtataaagctatacattgtgttcaaaatatgaaaccagaggagtatgctcataagattttggaattacagatgcaaagtataatggaaaagaaaatgaaaaccaagagaaatattgccaagcattacaagaataacccatcactaataactttcctttgcaaaaactgcagtgtgctagcctgttctggggaagatatccatgtaattgagaaaatgcatcacgtcaatatgaccccagaattcaaggaactttacattgtaagagaaaacaaaacactgcaaaagaagtgtgccgactatcaaataaatggtgaaatcatctgcaaatgtggccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgattaatgtattcattatgctacagaactgacataagaatcaa

SEQ ID NO: 17 - DNA, 3452 bp, Homo sapiens
  source 1..3452: mol_type = other DNA
acagaaaccaaagtcaggcaaactctgtaagaactgcctgacagaaagctggactcaaagctcctacccgagtgtgcagcaggatcgccccggtccgggaccccaggcgcacaccgcagagtccaaagtgccgcgcctgccggccgcacccgccggcctgagagccctgtggacaacctcgtcattgtcaggcacagagcggtagaccctgcttctctaagtgggcagcggacagcggcacgcacatttcacctgtcccgcagacaacagcaccatctgcttgggagaaccctctcccttctctgagaaagaaagatgtcgaatgggtattccacagacgagaatttccgctatctcatctcgtgcttcagggccagggtgaaaatgtacatccaggtggagcctgtgctggactacctgacctttctgcctgcagaggtgaaggagcagattcagaggacagtcgccacctccgggaacatgcaggcagttgaactgctgctgagcaccttggagaagggagtctggcaccttggttggactcgggaattcgtggaggccctccggagaaccggcagccctctggccgcccgctacatgaaccctgagctcacggacttgccctctccatcgtttgagaacgctcatgatgaatatctccaactgctgaacctccttcagcccactctggtggacaagcttctagttagagacgtcttggataagtgcatggaggaggaactgttgacaattgaagacagaaaccggattgctgctgcagaaaacaatggaaatgaatcaggtgtaagagagctactaaaaaggattgtgcagaaagaaaactggttctctgcatttctgaatgttcttcgtcaaacaggaaacaatgaacttgtccaagagttaacaggctctgattgctcagaaagcaatgcagagattgagaatttatcacaagttgatggtcctcaagtggaagagcaacttctttcaaccacagttcagccaaatctggagaaggaggtctggggcatggagaataactcatcagaatcatcttttgcagattcttctgtagtttcagaatcagacacaagtttggcagaaggaagtgtcagctgcttagatgaaagtcttggacataacagcaacatgggcagtgattcaggcaccgtgggaagtgattcagatgaagagaatgtggcagcaagagcatccccggagccagaactccagctcaggccttaccaaatggaagttgcccagccagccttggaagggaagaatatcatcatctgcctccctacagggagtggaaaaaccagagtggctgtttacattgccaaggatcacttagacaagaagaaaaaagcatctgagcctggaaaagttatagttcttgtcaataaggtactgctagttgaacagctcttccgcaaggagttccaaccatttttgaagaaatggtatcgtgttattggattaagtggtgatacccaactgaaaatatcatttccagaagttgtcaagtcctgtgatattattatcagtacagctcaaatccttgaaaactccctcttaaacttggaaaatggagaagatgctggtgttcaattgtcagacttttccctcattatcattgatgaatgtcatcacaccaacaaagaagcagtgtataataacatcatgaggcattatttgatgcagaagttgaaaaacaatagactcaagaaagaaaacaaaccagtgattccccttcctcagatactgggactaacagcttcacctggtgttggaggggccacgaagcaagccaaagctgaagaacacattttaaaactatgtgccaatcttgatgcatttactattaaaactgttaaagaaaaccttgatcaactgaaaaaccaaatacaggagccatgcaagaagtttgccattgcagatgcaaccagagaagatccatttaaagagaaacttctagaaataatgacaaggattcaaacttattgtcaaatgagtccaatgtcagattttggaactcaaccctatgaacaatgggccattcaaatggaaaaaaaagctgcaaaagaaggaaatcgcaaagaacgtgtttgtgcagaacatttgaggaagtacaatgaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacagaatgaacaaaaagaagtcattagtaaatttcgcactggaaaaataaatctgcttatcgctaccacagtggcagaagaaggtctggatattaaagaatgtaacattgttatccgttatggtctcgtcaccaatgaaatagccatggtccaggcccgtggtcgagccagagctgatgagagcacctgcgtcctggttgctcacagtggttcaggagttatcgaacgtgagacagttaatgatttccgagagaagatgatgtataaagctatacattgtgttcaaaatatgaaaccagaggagtatgctcataagattttggaattacagatgcaaagtataatggaaaagaaaatgaaaaccaagagaaatattgccaagcatttcaagaataacccatcactaataactttcctttgcaaaaactgcagtgtgctagcctgttctggggaagatatccatgtaattgagaaaatgcatcacgtcaatatgaccccagaattcaaggaactttacattgtaagagaaaacaaaacactgcaaaagaagtgtgccgactatcaaataaatggtgaaatcatctgcaaatgtggccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgatt

SEQ ID NO: 18 - DNA, 2540 bp, Homo sapiens
  source 1..2540: mol_type = other DNA
cgccccgctgcccacctgcccgcctgcccacctgcccaggtgcgagtgcagccccgcgcgccggcctgagagccctgtggacaacctcgtcattgtcaggcacagagcggtagaccctgcttctctaagtgggcagcggacagcggcacgcacatttcacctgtcccgcagacaacagcaccatctgcttgggagaaccctctcccttctctgagaaagaaagatgtcgaatgggtattccacagacgagaatttccgctatctcatctcgtgcttcagggccagggtgaaaatgtacatccaggtggagcctgtgctggactacctgacctttctgcctgcagaggtgaaggagcagattcagaggacagtcgccacctccgggaacatgcaggcagttgaactgctgctgagcaccttggagaagggagtctggcaccttggttggactcgggaattcgtggaggccctccggagaaccggcagccctctggccgcccgctacatgaaccctgagctcacggacttgccctctccatcgtttgagaacgctcatgatgaatatctccaactgctgaacctccttcagcccactctggtggacaagcttctagttagagacgtcttggataagtgcatggaggaggaactgttgacaattgaagacagaaaccggattgctgctgcagaaaacaatggaaatgaatcaggtgtaagagagctactaaaaaggattgtgcagaaagaaaactggttctctgcatttctgaatgttcttcgtcaaacaggaaacaatgaacttgtccaagagttaacaggctctgattgctcagaaagcaatgcagagattgagaatttatcacaagttgatggtcctcaagtggaagagcaacttctttcaaccacagttcagccaaatctggagaaggaggtctggggcatggagaataactcatcagaatcatcttttgcagattcttctgtagtttcagaatcagacacaagtttggcagaaggaagtgtcagctgcttagatgaaagtcttggacataacagcaacatgggcagtgattcaggcaccatgggaagtgattcagatgaagagaatgtggcagcaagagcatccccggagccagaactccagctcaggccttaccaaatggaagttgcccagccagccttggaagggaagaatatcatcatctgcctccctacagggagtggaaaaaccagagtggctgtttacattgccaaggatcacttagacaagaagaaaaaagcatctgagcctggaaaagttatagttcttgtcaataaggtactgctagttgaacagctcttccgcaaggagttccaaccatttttgaagaaatggtatcgtgttattggattaagtggtgatacccaactgaaaatatcatttccagaagttgtcaagtcctgtgatattattatcagtacagctcaaatccttgaaaactccctcttaaacttggaaaatggagaagatgctggtgttcaattgtcagacttttccctcattatcattgatgaatgtcatcacaccaacaaagaagcagtgtataataacatcatgaggcattatttgatgcagaagttgaaaaacaatagactcaagaaagaaaacaaaccagtgattccccttcctcagatactgggactaacagcttcacctggtgttggaggggccacgaagcaagccaaagctgaagaacacattttaaaactatgtgccaatcttgatgcatttactattaaaactgttaaagaaaaccttgatcaactgaaaaaccaaatacaggagccatgcaagaagtttgccattgcagatgcaaccagagaagatccatttaaagagaaacttctagaaataatgacaaggattcaaacttattgtcaaatgagtccaatgtcagattttggaactcaaccctatgaacaatgggccattcaaatggaaaaaaaagctgcaaaagaaggaaatcgcaaagaacgtgtttgtgcagaacatttgaggaagtacaatgaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacaaaaaaaaaaaaaaa

SEQ ID NO: 19 - protein, 1025 aa, Homo sapiens
MSNGYSTDENFRYLISCFRARVKMYIQVEPVLDYLTFLPAEVKEQIQRTVATSGNMQAVELLLSTLEKGVWHLGWTREFVEALRRTGSPLAARYMNPELTDLPSPSFENAHDEYLQLLNLLQPTLVDKLLVRDVLDKCMEEELLTIEDRNRIAAAENNGNESGVRELLKRIVQKENWFSAFLNVLRQTGNNELVQELTGSDCSESNAEIENLSQVDGPQVEEQLLSTTVQPNLEKEVWGMENNSSESSFADSSVVSESDTSLAEGSVSCLDESLGHNSNMGSDSGTMGSDSDEENVAARASPEPELQLRPYQMEVAQPALEGKNIIICLPTGSGKTRVAVYIAKDHLDKKKKASEPGKVIVLVNKVLLVEQLFRKEFQPFLKKWYRVIGLSGDTQLKISFPEVVKSCDIIISTAQILENSLLNLENGEDAGVQLSDFSLIIIDECHHTNKEAVYNNIMRHYLMQKLKNNRLKKENKPVIPLPQILGLTASPGVGGATKQAKAEEHILKLCANLDAFTIKTVKENLDQLKNQIQEPCKKFAIADATREDPFKEKLLEIMTRIQTYCQMSPMSDFGTQPYEQWAIQMEKKAAKEGNRKERVCAEHLRKYNEALQINDTIRMIDAYTHLETFYNEEKDKKFAVIEDDSDEGGDDEYCDGDEDEDDLKKPLKLDETDRFLMTLFFENNKMLKRLAENPEYENEKLTKLRNTIMEQYTRTEESARGIIFTKTRQSAYALSQWITENEKFAEVGVKAHHLIGAGHSSEFKPMTQNEQKEVISKFRTGKINLLIATTVAEEGLDIKECNIVIRYGLVTNEIAMVQARGRARADESTYVLVAHSGSGVIEHETVNDFREKMMYKAIHCVQNMKPEEYAHKILELQMQSIMEKKMKTKRNIAKHYKNNPSLITFLCKNCSVLACSGEDIHVIEKMHHVNMTPEFKELYIVRENKALQKKCADYQINGEIICKCGQAWGTMMVHKGLDLPCLKIRNFVVVFKNNSTKKQYKKWVELPITFPNLDYSECCLFSDED

SEQ ID NO: 20 - protein, 986 aa, Homo sapiens
MSNGYSTDENFRYLISCFRARVKMYIQVEPVLDYLTFLPAEVKEQIQRTVATSGNMQAVELLLSTLEKGVWHLGWTREFVEALRRTGSPLAARYMNPELTDLPSPSFENAHDEYLQLLNLLQPTLVDKLLVRDVLDKCMEEELLTIEDRNRIAAAENNGNESGVRELLKRIVQKENWFSAFLNVLRQTGNNELVQELTGSDCSESNAEIENLSQVDGPQVEEQLLSTTVQPNLEKEVWGMENNSSESSFADSSVVSESDTSLAEGSVSCLDESLGHNSNMGSDSGTMGSDSDEENVAARASPEPELQLRPYQMEVAQPALEGKNIIICLPTGSGKTRVAVYIAKDHLDKKKKASEPGKVIVLVNKVLLVEQLFRKEFQPFLKKWYRVIGLSGDTQLKISFPEVVKSCDIIISTAQILENSLLNLENGEDAGVQLSDFSLIIIDECHHTNKEAVYNNIMRHYLMQKLKNNRLKKENKPVIPLPQILGLTASPGVGGATKQAKAEEHILKDPFKEKLLEIMTRIQTYCQMSPMSDFGTQPYEQWAIQMEKKAAKEGNRKERVCAEHLRKYNEALQINDTIRMIDAYTHLETFYNEEKDKKFAVIEDDSDEGGDDEYCDGDEDEDDLKKPLKLDETDRFLMTLFFENNKMLKRLAENPEYENEKLTKLRNTIMEQYTRTEESARGIIFTKTRQSAYALSQWITENEKFAEVGVKAHHLIGAGHSSEFKPMTQNEQKEVISKFRTGKINLLIATTVAEEGLDIKECNIVIRYGLVTNEIAMVQARGRARADESTYVLVAHSGSGVIEHETVNDFREKMMYKAIHCVQNMKPEEYAHKILELQMQSIMEKKMKTKRNIAKHYKNNPSLITFLCKNCSVLACSGEDIHVIEKMHHVNMTPEFKELYIVRENKALQKKCADYQINGEIICKCGQAWGTMMVHKGLDLPCLKIRNFVVVFKNNSTKKQYKKWVELPITFPNLDYSECCLFSDED

SEQ ID NO: 21 - protein, 468 aa, Homo sapiens [sequence omitted]

SEQ ID NO: 22 - protein, 772 aa, Homo sapiens
MSNGYSTDENFRYLISCFRARVKMYIQVEPVLDYLTFLPAEVKEQIQRTVATSGNMQAVELLLSTLEKGVWHLGWTREFVEALRRTGSPLAARYMNPELTDLPSPSFENAHDEYLQLLNLLQPTLVDKLLVRDVLDKCMEEELLTIEDRNRIAAAENNGNESGVRELLKRIVQKENWFSAFLNVLRQTGNNELVQELTGSDCSESNAEIENLSQVDGPQVEEQLLSTTVQPNLEKEVWGMENNSSESSFADSSVVSESDTSLAEGSVSCLDESLGHNSNMGSDSGTMGSDSDEENVAARASPEPELQLRPYQMEVAQPALEGKNIIICLPTGSGKTRVAVYIAKDHLDKKKKASEPGKVIVLVNKVLLVEQLFRKEFQPFLKKWYRVIGLSGDTQLKISFPEVVKSCDIIISTAQILENSLLNLENGEDAGVQLSDFSLIIIDECHHTNKEAVYNNIMRHYLMQKLKNNRLKKENKPVIPLPQILGLTASPGVGGATKQAKAEEHILKLCANLDAFTIKTVKENLDQLKNQIQEPCKKFAIADATREDPFKEKLLEIMTRIQTYCQMSPMSDFGTQPYEQWAIQMEKKAAKEGNRKERVCAEHLRKYNEALQINDTIRMIDAYTHLETFYNEEKDKKFAVIEDDSDEGGDDEYCDGDEDEDDLKKPLKLDETDRFLMTLFFENNKMLKRLAENPEYENEKLTKLRNTIMEQYTRTEESARGIIFTKTRQSAYALSQWITENEKFAEVGVKAHHLIGAGHSSEFKPMTQKKKK

SEQ ID NO: 23 - protein, 221 aa, Homo sapiens
MSNGYSTDENFRYLISCFRARVKMYIQVEPVLDYLTFLPAEVKEQIQRTVATSGNMQAVELLLSTLEKGVWHLGWTREFVEALRRTGSPLAARYMNPELTDLPSPSFENAHDEYLQLLNLLQPTLVDKLLVRDVLDKCMEEELLTIEDRNRIAAAENNGNESGVRELLKRIVQKENWFSAFLNVLRQTGNNELVQELTGSDCSESNAGICNFTEEDSSNSA

SEQ ID NO: 24 - DNA, 23 bp, synthetic construct
  misc_feature 1..23: note = gRNA Recognition Sequence
  source 1..23: mol_type = other DNA
gcaatggcaaacttcttgcatgg

SEQ ID NO: 25 - DNA, 23 bp, synthetic construct
  misc_feature 1..23: note = gRNA Recognition Sequence
  source 1..23: mol_type = other DNA
agatgcaaccagagaagtatggg

SEQ ID NO: 26 - DNA, 23 bp, synthetic construct
  misc_feature 1..23: note = gRNA Recognition Sequence
  source 1..23: mol_type = other DNA
cagatgcaaccagagaagtatgg

SEQ ID NO: 27 - DNA, 23 bp, synthetic construct
  misc_feature 1..23: note = gRNA Recognition Sequence
  source 1..23: mol_type = other DNA
tactacattcagtagaaagatgg

SEQ ID NO: 28 - DNA, 23 bp, synthetic construct
  misc_feature 1..23: note = gRNA Recognition Sequence
  source 1..23: mol_type = other DNA
tcaactgaaaaaccaaatacagg

SEQ ID NO: 29 - DNA, 23 bp, synthetic construct
  misc_feature 1..23: note = gRNA Recognition Sequence
  source 1..23: mol_type = other DNA
ttctctggttgcatctgcaatgg

SEQ ID NO: 30 - DNA, 23 bp, synthetic construct
  misc_feature 1..23: note = gRNA Recognition Sequence
  source 1..23: mol_type = other DNA
attcagtagaaagatggcaaagg

SEQ ID NO: 31 - DNA, 23 bp, synthetic construct
  misc_feature 1..23: note = gRNA Recognition Sequence
  source 1..23: mol_type = other DNA
acattaagcccatacttctctgg

SEQ ID NO: 32 - DNA, 23 bp, synthetic construct
  misc_feature 1..23: note = gRNA Recognition Sequence
  source 1..23: mol_type = other DNA
tcttgcatggctcctgtatttgg

SEQ ID NO: 33 - DNA, 23 bp, synthetic construct
  misc_feature 1..23: note = gRNA Recognition Sequence
  source 1..23: mol_type = other DNA
tttggtttttcagttgatcaagg

SEQ ID NO: 34 - DNA, 23 bp, synthetic construct
  misc_feature 1..23: note = gRNA Recognition Sequence
  source 1..23: mol_type = other DNA
tttaaataatatttttcagatgg